(19) United States
(12) Patent Application Publication
(10) Pub. No.: US 2024/0081985 A1
(43) Pub. Date: Mar. 14, 2024

(54) MECHANICALLY-EXPANDABLE PROSTHETIC HEART VALVES, DELIVERY APPARATUS, AND METHODS

(71) Applicant: EDWARDS LIFESCIENCES CORPORATION, Irvine, CA (US)

(72) Inventors: Noam Miller, Givatayim (IL); Noam Nir, Pardes-Hanna (IL); Michael Bukin, Pardes Hanna (IL)

(21) Appl. No.: 18/509,778

(22) Filed: Nov. 15, 2023

Related U.S. Application Data
(63) Continuation of application No. PCT/US2022/031257, filed on May 27, 2022.
(60) Provisional application No. 63/194,285, filed on May 28, 2021.

(51) Int. Cl. A61F 2/24

(52) U.S. Cl. CPC A61F 2/2418; A61F 2/243; A61F 2220/0091

(57) ABSTRACT

Various examples of prosthetic heart valves, delivery apparatus, and methods of using such are disclosed herein. The prosthetic heart valves comprise frames that are movable between a radially-compressed configuration and one or more radially-expanded configurations. The frames of the prosthetic heart valves include actuation members and locking mechanisms configured to secure the frame in one or more radially-expanded configurations. In some examples, the actuation members and locking members are integrally formed with the frame. The frames of the prosthetic heart valves can be coupled to one or more shafts of a delivery apparatus via a threaded connection or a non-threaded connection.

### CROSS-REFERENCE TO RELATED APPLICATIONS

This application is a continuation of International Patent Application No. PCT/US2022/031257, filed May 27, 2022, which claims the benefit of U.S. Provisional Patent Application No. 63/194,285, filed May 28, 2021. The related applications are incorporated by reference herein.

### FIELD

The present disclosure relates to implantable, mechanically-expandable prosthetic devices, such as prosthetic heart valves, and to delivery apparatus and methods for implanting prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Prosthetic heart valves that rely on a mechanical actuator for expansion can be referred to as “mechanically-expandable” prosthetic heart valves. Mechanically-expandable prosthetic heart valves can provide one or more advantages over self-expandable and balloon-expandable prosthetic heart valves. For example, mechanically-expandable prosthetic heart valves can be expanded to various diameters. Mechanically-expandable prosthetic heart valves can also be compressed after an initial expansion (e.g., for repositioning and/or retrieval).

Despite these advantages, mechanically-expandable prosthetic heart valves can present several challenges. For example, it can be difficult to retain the prosthetic heart valve in a desired expanded state and/or to have expansion and/or locking mechanisms that are manufacturable, usable, and reliable. These difficulties can be compounded by the small size of the components. It can also be difficult to couple/release a mechanically-expandable prosthetic heart valve to/from the delivery apparatus. Accordingly, there is a need for improved mechanically-expandable prosthetic heart valves, as well as delivery apparatus and methods for implanting mechanically-expandable prosthetic heart valves.

### SUMMARY

Described herein are mechanically-expandable prosthetic heart valves, delivery apparatus, and methods for implanting mechanically-expandable prosthetic heart valves. The disclosed prosthetic heart valves, delivery apparatus, and methods can, for example, provide robust and reliable actuation and locking when the prosthetic heart valve is moved from a radially-compressed configuration to one or more radially-expanded configurations. The disclosed devices and methods are also dependable and easy to use, for example, when coupling/releasing a mechanically-expandable prosthetic heart valve to/from a delivery apparatus. As such, the devices and methods disclosed herein can, among other things, overcome one or more of the deficiencies of typical mechanically-expandable prosthetic heart valves and their delivery apparatus.

In one example, a prosthetic heart valve comprises a frame that includes a plurality of struts, an actuation member, a locking mechanism, a first end portion, and a second end portion. The frame is movable from a radially-compressed configuration to a plurality of radially-expanded configurations. The plurality of struts and the locking mechanism are integrally formed as a unitary component. The actuation member extends from the first end portion of the frame toward the second end portion of the frame. The locking mechanism is disposed at the second end portion of the frame and is configured to receive the actuation member. The locking mechanism is configured to selectively engage the actuation member such that the actuation member is movable in a first direction relative to the frame when the frame is in a first radially-expanded configuration, thereby allowing further radial expansion of the frame from the first radially-expanded configuration to a second radially-expanded configuration, and such that the actuation member is prevented from moving in a second direction relative to the frame when the frame is in the first radially-expanded configuration, thereby preventing the frame from moving from the first radially-expanded configuration to the radially-compressed configuration.

In another example, a prosthetic heart valve comprises a frame including a plurality of struts, a plurality of actuation members, a plurality of locking mechanisms, a first end portion and a second end portion. The frame is movable from a radially-compressed configuration to a plurality of radially-expanded configurations. The plurality of struts and the plurality of locking mechanisms are integrally formed as a unitary component. The plurality of actuation members extends from the first end portion of the frame toward the second end portion of the frame, each actuation member of the plurality of actuation members being spaced circumferentially relative to an adjacent actuation member of the plurality of actuation members. The plurality of locking mechanisms is disposed at the second end portion of the frame, each locking mechanism of the plurality of locking mechanisms being spaced circumferentially relative to an adjacent locking mechanism of the plurality of actuation members and configured to receive a respective actuation member of the plurality of actuation members and to selectively engage the respective actuation member such that the respective actuation member is movable in a first direction relative to the frame when the frame is in the first radially-expanded configuration, thereby allowing the frame to radially expand from the first radially-expanded configuration to a second radially-expanded configuration, and such that the respective actuation member is prevented from moving in a second direction relative to the frame when the frame is in the first radially-expanded configuration, thereby preventing the frame from moving from the first radially-expanded configuration to the radially-compressed configuration.

In another example, a method of implanting a prosthetic heart valve is provided. The method comprises inserting a prosthetic heart valve into a patient's vasculature, the prosthetic heart valve releasably coupled to a distal end portion of a delivery apparatus and in a radially-compressed configuration. The method further comprises advancing the prosthetic heart valve through the patient's vasculature to an implantation location, expanding the prosthetic heart valve to a radially-expanded configuration by applying an axially-compressive force on the prosthetic heart valve with the delivery apparatus, and locking the prosthetic heart valve in the radially-expanded configuration by engaging an actuation member and a locking mechanism of the prosthetic heart valve such that the prosthetic heart valve is prevented from moving from the radially-expanded configuration to the radially-compressed configuration, wherein the locking mechanism is integrally formed with a frame of the prosthetic heart valve.

In another example, a prosthetic heart valve comprises a frame and a valve structure. The frame comprising a plurality of struts, an actuation member, a locking mechanism, a first end portion and a second end portion. The frame is movable from a radially-compressed configuration and a radially-expanded configuration. The plurality of struts and the locking mechanism are integrally formed as a unitary component. The actuation member extends from the first end portion of the frame toward the second end portion of the frame. The locking mechanism is disposed at the second end portion of the frame and comprises a channel and a retention element. The channel is configured to receive the actuation member. The retention element is configured to selectively engage the actuation member such that the actuation member is movable in a first direction relative to the channel as the frame moves from the radially-compressed configuration to the radially-expanded configuration and such that the actuation member is prevented from moving in a second direction relative to the channel when the frame is in the radially-expanded configuration, thereby preventing the frame from moving from the radially-expanded configuration to the radially-compressed configuration. The valve structure coupled to the frame and comprising a plurality of leaflets configured for allowing blood to flow in an antegrade direction and to restrict blood from flowing in a retrograde direction.

In another example, a method of implanting a prosthetic heart valve is provided. The method comprises inserting a prosthetic heart valve into a patient's vasculature, the prosthetic heart valve releasably coupled to a distal end portion of a delivery apparatus and in a radially-compressed configuration, advancing the prosthetic heart valve through the patient's vasculature to an implantation location, expanding the prosthetic heart valve to a radially-expanded configuration by applying an axially-compressive force on the prosthetic heart valve with the delivery apparatus, and locking the prosthetic heart valve in the radially-expanded configuration by engaging an actuation member and a locking mechanism of the prosthetic heart valve such that the prosthetic heart valve is prevented from moving from the radially-expanded configuration to the radially-compressed configuration, wherein the locking mechanism is integrally formed with a frame of the prosthetic heart valve.

In another example, a prosthetic heart valve comprises a frame including a plurality of struts, an actuation member, a locking mechanism, a first end portion, and a second end portion. The frame is movable from a radially-compressed configuration to a plurality of radially-expanded configurations, which are radially larger than the radially-compressed configuration. The plurality of struts and the locking mechanism are integrally formed as a unitary component. The actuation member extends from the first end portion of the frame toward the second end portion of the frame. The locking mechanism is disposed at the second end portion of the frame and comprises a lumen and a retention element. The lumen is configured to receive the actuation member. The retention element comprises an aperture configured to receive the actuation member. The retention element is biased to a first configuration and is movable between the first configuration and a second configuration. When the retention element is in the first configuration, the aperture of the retention element is misaligned with the actuation member such that one or more portions of the retention element defining the aperture engage the actuation member, thereby preventing the actuation member from moving in a first direction relative to the retention element and securing the frame in one of the radially-expanded configurations. When the retention element is in the second configuration, the aperture of the retention element is aligned with the actuation member, thereby allowing the actuation member to move in a second direction relative to the retention element and allowing the frame to move from the radially-compressed configuration to the plurality of radially-expanded configurations.

In another example, a prosthetic heart valve comprises a frame with a plurality of struts, an actuation member, a locking mechanism, a first end portion and a second end portion. The frame is movable from a radially-compressed configuration and a radially-expanded configuration, which is radially larger than the radially-compressed configuration. The plurality of struts and the locking mechanism are integrally formed as a unitary component. The locking mechanism is disposed at the first end portion of the frame and comprises a slot, a first retention element, and a second retention element. The first retention element extends from a first side of the slot. The second retention element extends from a second side of the slot. In the radially-expanded configuration, the actuation member is disposed in the slot, the first retention element engages a first segment of the actuation member, and the second retention element engages a second segment of the actuation member, thereby preventing the frame from moving from the radially-expanded configuration to the radially-compressed configuration.

In another example, a method of implanting a prosthetic heart valve is provided. The method comprises inserting a prosthetic heart valve into a patient's vasculature, the prosthetic heart valve releasably coupled to a distal end portion of a delivery apparatus and in a radially-compressed configuration, advancing the prosthetic heart valve through the patient's vasculature to an implantation location, expanding the prosthetic heart valve to a radially-expanded configuration by applying an axially-compressive force on the prosthetic heart valve with the delivery apparatus, and locking the prosthetic heart valve in the radially-expanded configuration by engaging an actuation member and a locking mechanism of the prosthetic heart valve such that the prosthetic heart valve is prevented from moving from the radially-expanded configuration to the radially-compressed configuration, wherein the locking mechanism is integrally formed with a frame of the prosthetic heart valve and comprises a first retention element contacting a first segment of the actuation member and a second retention element contacting a second segment of the actuation member.

In another example, a prosthetic heart valve comprises a frame with a plurality of struts, an actuation member, a locking mechanism, a first end portion, and a second end portion. The frame is movable from a radially-compressed configuration to a plurality of radially-expanded configurations, which are radially larger than the radially-compressed configuration. The plurality of struts and the locking mechanism are integrally formed as a unitary component. The actuation member extends from the first end portion of the frame toward the second end portion of the frame. The locking mechanism is disposed at the second end portion of the frame and extends toward the first end portion of the frame, the locking mechanism including a first aperture and a second aperture axially spaced apart from each other, and each configured to receive the actuation member. The locking mechanism is biased to a first configuration and is movable between the first configuration and a second configuration. When the locking mechanism is in the first configuration, the first aperture and the second aperture of the locking mechanism are misaligned with the actuation member such that one or more portions of the locking mechanism defining the first aperture and the second aperture engage the actuation member, thereby preventing the actuation member from moving in a first direction relative to the locking mechanism and securing the frame in one of the radially-expanded configurations. When the locking mechanism is in the second configuration, the first aperture and the second aperture of the locking mechanism are aligned with the actuation member, thereby allowing the actuation member to move in a second direction relative to the locking mechanism and allowing the frame to move from the radially-compressed configuration to the plurality of radially-expanded configurations.

In another example, a prosthetic heart valve comprises a frame comprising a plurality of struts, an actuation member, a first locking mechanism, a second locking mechanism, a first end portion, and a second end portion. The frame is movable from a radially-compressed configuration to a plurality of radially-expanded configurations, which are radially larger than the radially-compressed configuration. The plurality of struts, the first locking mechanism, and the second locking mechanism are integrally formed as a unitary component. The actuation member extends from the first end portion of the frame toward the second end portion of the frame. The first locking mechanism and the second locking mechanism are disposed at the second end portion of the frame and extend toward the first end portion of the frame, the first locking mechanism including a first aperture, the second locking mechanism including a second aperture, the first aperture and the second aperture axially spaced apart from each other and configured to receive the actuation member. The first locking mechanism and the second locking mechanism are biased to a first configuration and are movable between the first configuration and a second configuration. When the first locking mechanism and the second locking mechanism are in the first configuration, the first aperture and the second aperture are misaligned with the actuation member such that the first locking mechanism and the second locking mechanism engage the actuation member, thereby preventing the actuation member from moving in a first direction relative to the first locking mechanism and the second locking mechanism and securing the frame in one of the radially-expanded configurations. When the first locking mechanism and the second locking mechanism are in the second configuration, the first aperture and the second aperture are aligned with the actuation member, thereby allowing the actuation member to move in a second direction relative to the first locking mechanism and the second locking mechanism and allowing the frame to move from the radially-compressed configuration to the plurality of radially-expanded configurations.

In another example, a prosthetic heart valve comprises a frame including a plurality of struts, an actuation member, a locking mechanism, a first end portion, and a second end portion. The frame is movable from a radially-compressed configuration to a plurality of radially-expanded configurations, which are radially larger than the radially-compressed configuration. The actuation member extends from the first end portion of the frame toward the second end portion of the frame. The locking mechanism comprises a window and a locker disc, the window formed in a non-pivoting strut of the plurality of struts disposed at the second end portion of the frame and comprising a support shoulder, and the locker disc comprising a first side portion, a second side portion, and an opening, the first side portion of the locker disc disposed on the support shoulder, the second side portion spaced from the support shoulder, and the opening configured for receiving the actuation member. The locker disc is pivotable about the support shoulder between a locked position and an unlocked position. When the locker disc is in the locked position, the opening of the locker disc is misaligned with the actuation member and the locker disc engages the actuation member such that the actuation member is prevented from moving in a first direction relative to the window, thereby securing the frame in one of the radially-expanded configurations. When the locker disc is in the unlocked position, the opening of the locker disc is aligned with the actuation member such that the actuation member can move in a second direction relative to the window, thereby allowing the frame to move from the radially-compressed configuration to the plurality of radially-expanded configurations.

In another example, a prosthetic heart valve comprises a frame comprising a plurality of struts, an actuation member, a locking mechanism, a first end portion, and a second end portion. The frame is movable from a radially-compressed configuration to a plurality of radially-expanded configurations, which are radially larger than the radially-compressed configuration. The actuation member extends from the first end portion of the frame toward the second end portion of the frame. The locking mechanism comprises a window and a retention element, the window formed in a non-pivoting strut of the plurality of struts disposed at the second end portion of the frame and comprising a shoulder, the retention element disposed in the window and configured to engage the actuation member such that the actuation member is movable in a first direction relative to the retention element and prevented from moving in a second direction relative to the retention element, the first direction corresponding to radial expansion of the frame, and the second direction corresponding to radial compression of the frame.

In another example, a prosthetic heart valve comprises a frame with a plurality of struts, an actuation member, a locking mechanism, a first end portion, and a second end portion. The frame is movable from a radially-compressed configuration to a plurality of radially-expanded configurations, which are radially larger than the radially-compressed configuration. The actuation member extends from the first end portion of the frame toward the second end portion of the frame. The locking mechanism comprises a chamber and a retention member, the chamber formed in a non-pivoting strut of the plurality of struts disposed at the second end portion of the frame and at least partially defined by one or more ramped side walls of the non-pivoting strut, the retention member disposed in the chamber and comprising a base segment and one or more arms extending from the base segment, the base segment comprising a lumen configured for receiving the actuation member, and the one or more arms configured to engage the actuation member. The retention member is axially movable within the chamber between a locked position and an unlocked position. In the locked position, the one or more arms of the retention member contact the one or more ramped side walls of the non-pivoting strut, thereby securing the one or more arms of the retention member against the actuation member and preventing the actuation member from moving axially relative to the retention member toward the first end portion of the frame. In the unlocked position, the actuation member is released from the one or more arms of the retention member such that the actuation member is axially movable relative to the retention member toward the second end portion of the frame.

In another example, a prosthetic heart valve comprises a frame having a plurality of struts, an actuation member, a locking mechanism, a first end portion, and a second end portion. The frame is movable from a radially-compressed configuration to a plurality of radially-expanded configurations, which are radially larger than the radially-compressed configuration. The actuation member extends from the first end portion of the frame toward the second end portion of the frame. The locking mechanism comprises a chamber and one or more retention members, the chamber formed in a non-pivoting strut of the plurality of struts disposed at the second end portion of the frame and at least partially defined by one or more curved surfaces of the non-pivoting strut, the one or more retention members disposed in the chamber, each of the one or more retention members comprising an arm portion and a cam portion, the arm portion fixedly coupled to the non-pivoting strut in which the chamber is formed and the cam portion extending from the arm portion and configured to engage the actuation member. The one or more retention members are axially movable within the chamber between a locked position and an unlocked position. In the locked position, the cam portion of each of the one or more retention members contacts a respective curved surface of the non-pivoting strut, which secures the cam portion of each of the one or more retention members against the actuation member and restricts the actuation member from moving axially relative to the one or more retention members toward the first end portion of the frame. In the unlocked position, the cam portion of each of the one or more retention members is axially spaced from the respective curved surface of the non-pivoting strut, which allows the actuation member to move axially relative to the one or more retention members toward the second end portion of the frame.

In another example, a method of implanting a prosthetic heart valve comprises inserting a prosthetic heart valve into a patient's vasculature, the prosthetic heart valve releasably coupled to a distal end portion of a delivery apparatus and in a first radially-compressed configuration, advancing the prosthetic heart valve through the patient's vasculature to an implantation location, expanding the prosthetic heart valve to a first radially-expanded configuration by moving an actuation member of the prosthetic heart valve in a first axial direction relative to a locking mechanism of the prosthetic heart valve, wherein the actuation member is restricted from rotating relative to the locking mechanism during the expansion of the prosthetic heart valve to the first radially-expanded configuration, locking the prosthetic heart valve in the first radially-expanded configuration by engaging the actuation member with the locking mechanism of the prosthetic heart valve such that the actuation member is restricted from moving in a second axial direction relative to the locking mechanism, and compressing the prosthetic heart valve from the first radially-expanded configuration to a second radially-compressed configuration, which is larger than the first radially-compressed configuration, wherein compressing the prosthetic heart valve includes rotating the actuation member in a first rotational direction relative to the locking mechanism.

In another example, a prosthetic heart valve comprises a plurality of pivoting struts, a plurality of non-pivoting struts, an actuation member, and a locking mechanism. The plurality of non-pivoting struts includes a first non-pivoting strut and a second non-pivoting strut axially spaced apart from each other. The plurality of non-pivoting struts is fixedly coupled to the plurality of pivoting struts. The second non-pivoting strut includes a lumen. The actuation member fixedly coupled to the first non-pivoting strut and extending from the first non-pivoting strut and into the lumen of the second non-pivoting strut. The locking mechanism comprises a chamber and a retention member. The chamber is formed in the second non-pivoting strut, intersects with the lumen of the second non-pivoting strut, and is configured to receive the retention member therein. The prosthetic heart valve is radially expandable from a radially-compressed state to a radially-expanded state by moving the actuation member in a first axial direction relative to the second non-pivoting strut. The prosthetic heart valve is radially compressible from the radially-expanded state to the radially-compressed state by moving the actuation member in a second axial direction relative to the second non-pivoting strut. The locking mechanism is movable within the chamber from a locked position to an unlocked position. In the locked position, the retention member engages the actuation member, prevents the actuation member from moving in the second axial direction to the second non-pivoting strut, and allows the actuation member to move in the first axial direction relative to the second non-pivoting strut. In unlocked position, the retention member is disengaged from the actuation member and movable in the first axial direction relative to the second non-pivoting strut.

In another example, a delivery apparatus for a prosthetic implant comprises a handle, a locking shaft, and an actuation shaft. The locking shaft having a proximal end portion and a distal end portion. The proximal end portion of the locking shaft is movably coupled to the handle. The distal end portion of the locking shaft is configured to be inserted through a lumen of a prosthetic implant having a diameter and to be movable between a straight configuration and a flared configuration. In the straight configuration, the distal end portion of the locking shaft has a first outer diameter and a first inner diameter, the first outer diameter being less than the diameter of the lumen. In the flared configuration, the distal end portion of the locking shaft has a second outer diameter and a second inner diameter, the second outer diameter being greater than the diameter of the lumen. The actuation shaft extending coaxially through the locking shaft and having a proximal end portion and a distal end portion, wherein the proximal end portion of the actuation shaft is movably coupled to the handle. The distal end portion of the actuation shaft has an outer diameter that is less than the diameter of the lumen, less than the second inner diameter of the locking shaft, and greater than the first inner diameter of the locking shaft. The actuation shaft and the locking shaft are axially movable relative to each other between an engaged state and a disengaged state. In the engaged state, the distal end portion of the locking shaft is in the flared configuration and the distal end portion of the actuation shaft is at least partially disposed within the locking shaft such that an outer surface of the actuation shaft contacts an inner surface of the distal end portion of the locking shaft. The actuation shaft secures the locking shaft in the flared configuration. The locking shaft prevents the actuation shaft from moving proximally relative to the locking shaft. In the disengaged state, the distal end portion of the actuation shaft is positioned distal relative to the distal end portion of the locking shaft such that the outer surface of the actuation shaft is spaced from the inner surface of the distal end portion of the locking shaft. The locking shaft can move from the flared configuration to the straight configuration. The locking shaft can move proximally relative to the actuation shaft.

In another example, a method of implanting a prosthetic implant comprises positioning an actuation shaft of a delivery apparatus through a lumen of a prosthetic implant such that a distal end portion of the actuation shaft is disposed distal to a distal end of the lumen, positioning a locking shaft of the delivery apparatus over the actuation shaft and through the lumen of the prosthetic implant such that a distal end portion of the locking shaft is disposed distal to the distal end of the lumen and proximal to the distal end portion of the actuation shaft, wherein the distal end portion of the locking shaft comprises a flange that flares radially and contacts the prosthetic implant, moving the actuation shaft proximally relative to the locking shaft such that the distal end portion of the actuation shaft contacts the flange of the locking shaft such that the actuation shaft and the locking shaft are restricted from moving proximally relative to the prosthetic implant, inserting the prosthetic implant into a patient's body together with the distal end portion of the actuation shaft and the distal end portion of the locking shaft, advancing the prosthetic implant to an implantation location with the patient's body, expanding the prosthetic implant from a radially-compressed configuration to a radially-expanded configuration by applying an axially-compressive force on the prosthetic implant via the actuation shaft, locking the prosthetic implant in the radially-expanded configuration with a locking mechanism of the prosthetic implant, and releasing the prosthetic implant from the delivery apparatus by moving the distal end portion of the actuation shaft distally relative to the locking shaft, moving the locking shaft proximally relative to the prosthetic implant such the locking shaft is withdrawn from the lumen, and moving the actuation shaft proximally relative to the prosthetic implant such that the actuation shaft is withdrawn from the lumen.

In another example, a frame for a prosthetic heart valve comprises a plurality of pivoting struts, a plurality of non-pivoting struts coupled to the plurality of pivoting struts, an actuation member coupled to a first non-pivoting strut of the plurality of non-pivoting struts, and a locking mechanism coupled to a second non-pivoting strut of the plurality of non-pivoting struts. The locking mechanism is integrally formed as a single, unitary component with the plurality of pivoting struts and the plurality of non-pivoting struts. The frame is movable from a radially-compressed state to a radially-expanded state. In the radially-compressed state, the actuation member is axially spaced from the locking mechanism. In the radially-expanded state, locking mechanism engages the actuation member and prevents the frame from moving from the radially-expanded state to the radially-compressed state.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 depicts a perspective view of a mechanically-expandable prosthetic heart valve, according to one example.

FIG. 2A depicts a partial side view of a frame of the prosthetic heart valve of FIG. 1, depicting the frame in a radially-expanded configuration.

FIG. 2B depicts a partial side view of the frame of the prosthetic heart valve of FIG. 1, depicting the frame in a partially radially-expanded configuration.

FIG. 2C depicts a partial side view of the frame of the prosthetic heart valve of FIG. 1, depicting the frame in a radially-compressed configuration.

FIG. 3 depicts a partial side view of a delivery apparatus for a prosthetic heart valve, according to one example.

FIG. 4 depicts a cross-sectional view of the delivery apparatus of FIG. 3, taken from the perspective indicated by the line 4-4 in FIG. 3.

FIG. 5 depicts a detail view of the delivery apparatus of FIG. 3, depicting the portion indicated by the area 5 in FIG. 3.

FIG. 6A depicts a partial side view of a delivery assembly comprising the frame of the prosthetic heart valve of FIG. 1 and a distal end portion the delivery apparatus of FIG. 3, depicting the frame of the prosthetic heart valve in a radially-expanded configuration.

FIG. 6B depicts a partial side view of the delivery assembly of FIG. 6A, depicting the frame of the prosthetic heart valve in a radially-compressed configuration.

FIG. 6C depicts a partial side view of the delivery assembly of FIG. 6A, depicting a delivery capsule of the delivery apparatus, which comprises the frame of the prosthetic heart valve disposed therein in the radially-compressed configuration.

FIG. 7A depicts a delivery assembly of FIG. 6A inserted into a heart (shown in partial cross-section) using a transfemoral delivery procedure, depicting the frame of the prosthetic heart valve disposed within the delivery capsule of delivery apparatus.

FIG. 7B depicts a delivery assembly of FIG. 6A inserted into the heart using the transfemoral delivery procedure, depicting the frame of the prosthetic heart valve partially exposed from the delivery capsule of delivery apparatus.

FIG. 7C depicts a delivery assembly of FIG. 6A inserted into the heart using the transfemoral delivery procedure, depicting the frame of the prosthetic heart valve fully exposed from the delivery capsule of delivery apparatus and in a radially-compressed configuration.

FIG. 7D depicts a delivery assembly of FIG. 6A inserted into the heart using the transfemoral delivery procedure, depicting the frame of the prosthetic heart valve fully exposed from the delivery capsule of delivery apparatus and in a radially-expanded configuration.

FIG. 7E depicts a delivery assembly of FIG. 6A inserted into the heart using the transfemoral delivery procedure, depicting the frame of the prosthetic heart valve fully exposed from the delivery capsule of delivery apparatus, in the radially-expanded configuration, and released from the delivery apparatus.

FIG. 8 depicts a partial perspective view of a frame of a prosthetic heart valve, according to another example.

FIG. 9A depicts a detail view of a second end portion of the frame of FIG. 8, depicting retention elements of a locking mechanism in a lateral configuration.

FIG. 9B depicts a detail view of the second end portion of the frame of FIG. 8, depicting retention elements of a locking mechanism in an angled configuration.

FIG. 10 depicts the frame of FIG. 8 releasably coupled to the distal end portion of the delivery apparatus of FIG. 3 to form a delivery assembly.

FIG. 11 depicts a detail view of the delivery assembly of FIG. 10, depicting the frame in a locked configuration.

FIG. 12 depicts a partial perspective view of a frame of a prosthetic heart valve, according to another example, releasably coupled to the delivery apparatus of FIG. 3.

FIG. 13A depicts a detail view of the frame of FIG. 12, depicting a locking mechanism of the frame in an angled, locked configuration.

FIG. 13B depicts a cross-sectional view of the frame of FIG. 12, taken from the perspective indicated by line 13B-13B in FIG. 13A and depicting the locking mechanism of the frame in the angled, locked configuration.

FIG. 14A depicts a detail view of the frame of FIG. 12, depicting the locking mechanism of the frame in a lateral, unlocked configuration.

FIG. 14B depicts a cross-sectional view of the frame of FIG. 12, taken from the perspective indicated by line 14B-14B in FIG. 14A and depicting the locking mechanism of the frame in the lateral, unlocked configuration.

FIG. 15 depicts a partial perspective view of a frame of a prosthetic heart valve, according to another example, depicting the frame in a locked configuration.

FIG. 16 depicts a detail view of the frame of FIG. 15, depicting the frame in the locked configuration.

FIG. 17A depicts a partial perspective view of the frame of FIG. 15, depicting the frame in a radially-compressed and unlocked configuration and releasably coupled to the delivery apparatus of FIG. 3.

FIG. 17B depicts a partial perspective view of the frame of FIG. 15, depicting the frame in a partially radially-expanded and unlocked configuration and releasably coupled to the delivery apparatus of FIG. 3.

FIG. 17C depicts a partial perspective view of the frame of FIG. 15, depicting the frame in a first radially-expanded and locked configuration and releasably coupled to the delivery apparatus of FIG. 3.

FIG. 17D depicts a partial perspective view of the frame of FIG. 15, depicting the frame in a second radially-expanded and locked configuration and releasably coupled to the delivery apparatus of FIG. 3.

FIG. 18 depicts a partial perspective view of a frame of a prosthetic heart valve, according to another example, depicting the frame in a locked configuration and releasably coupled to the delivery apparatus of FIG. 3.

FIG. 19A depicts a detail view of the frame of FIG. 18, depicting a locking mechanism of the frame in the locked configuration.

FIG. 19B depicts a cross-sectional view of the frame of FIG. 18, taken from the perspective indicated by line 19B-19B in FIG. 19A and depicting the locking mechanism of the frame in the locked configuration.

FIG. 20A depicts a detail view of the frame of FIG. 18, depicting a locking mechanism of the frame in an unlocked configuration.

FIG. 20B depicts a cross-sectional view of the frame of FIG. 18, taken from the perspective indicated by line 20B-20B in FIG. 20A and depicting the locking mechanism of the frame in the unlocked configuration.

FIG. 21 depicts a partial perspective view of a frame of a prosthetic heart valve, according to another example, depicting the frame in an unlocked configuration and releasably coupled to the delivery apparatus of FIG. 3.

FIG. 22A depicts a detail view of the frame of FIG. 22, depicting a locking mechanism of the frame in a locked configuration.

FIG. 22B depicts a detail view of the frame of FIG. 22, depicting the locking mechanism of the frame in the unlocked configuration.

FIG. 23 depicts a partial perspective view of a frame of a prosthetic heart valve, according to another example, depicting the frame in a locked configuration and releasably coupled to the delivery apparatus of FIG. 3.

FIG. 24 depicts a detail side view of the frame of FIG. 23, depicting a locking mechanism of the frame in a locked configuration.

FIG. 25 depicts a detail perspective view of the frame of FIG. 23, depicting a locking mechanism of the frame in a locked configuration.

FIG. 26A depicts a side elevation view of a locker disc of the frame of FIG. 23, depicting the locker disc in a curved configuration.

FIG. 26B depicts a top plan view of the locker disc of the frame of FIG. 23, depicting the locker disc in the curved configuration.

FIG. 27A depicts a side elevation view of a locker disc, according to one example and which can be used, for example, with the frame of FIG. 23 in lieu of the locker disc depicted in FIG. 26A-26B.

FIG. 27B depicts a top plan view of the locker disc of FIG. 27A.

FIG. 28A depicts a partial side view of the frame of FIG. 23, depicting the frame in a radially-compressed and locked configuration.

FIG. 28B depicts a partial side view of the frame of FIG. 23, depicting the frame in a partially radially-expanded and unlocked configuration.

FIG. 28C depicts a partial side view of the frame of FIG. 23, depicting the frame in a radially-expanded and locked configuration.

FIG. 29A depicts a side view of a locking mechanism of a frame, according to one example, depicting the locking mechanism in a locked configuration.

FIG. 29B depicts a side view of the locking mechanism of FIG. 29A, depicting the locking mechanism in an unlocked configuration.

FIG. 30A depicts a perspective view of a locking mechanism of a frame, according to one example, depicting the locking mechanism in a locked configuration.

FIG. 30B depicts a perspective view of the locking mechanism of FIG. 30A, depicting the locking mechanism in an unlocked configuration.

FIG. 31A depicts a perspective view of a locking mechanism of a frame, according to one example, depicting the locking mechanism in a locked configuration.

FIG. 31B depicts a perspective view of the locking mechanism of FIG. 31A, depicting the locking mechanism in an unlocked configuration.

FIG. 32A depicts a perspective view of a locking mechanism of a frame, according to one example, depicting the locking mechanism in a locked configuration.

FIG. 32B depicts a perspective view of the locking mechanism of FIG. 32A, depicting the locking mechanism in an unlocked configuration.

FIG. 33 depicts a partial side view of a frame, according to one example.

FIG. 34 depicts a detail perspective view of a locking mechanism of the frame of FIG. 33, depicting the portion indicated by the area 34 in FIG. 33.

FIG. 35 depicts a detail side view of a locking mechanism, according to one example and which can be used, for example, with the frame of FIG. 33 in lieu of the locking mechanism depicted in FIG. 34.

FIG. 36 depicts a detail perspective view of the locking mechanism of FIG. 35.

FIG. 37 depicts a detail side view of a locking mechanism, according to one example and which can be used, for example, with the frame of FIG. 33 in lieu of the locking mechanism depicted in FIG. 34.

FIG. 38 depicts a partial side view of the frame of FIG. 33 having an alternative locking mechanism.

FIG. 39 depicts a detail perspective view of the frame of FIG. 38.

FIG. 40A is a detail side view of the locking mechanism of FIG. 38, depicting the locking mechanism in a locked state.

FIG. 40B is a detail side view of the locking mechanism of FIG. 38, depicting the locking mechanism in an unlocked state.

FIG. 41 is a detail side view of the locking mechanism of FIG. 38, depicting the locking mechanism in the unlocked state and having an optional biasing member.

FIG. 42A is a detail side view of a locking mechanism, according to another example and which can be used, for example, with the frame of FIG. 33 in lieu of the locking mechanism depicted in FIG. 34, depicting the locking mechanism in a locked state.

FIG. 42B is a detail side view of the locking mechanism of FIG. 42A, depicting the locking mechanism in an unlocked state.

FIG. 43 is a detail perspective view of a locking mechanism, according to another example and which can be used, for example, with the frame of FIG. 33 in lieu of the locking mechanism depicted in FIG. 34.

FIG. 44A is a detail side view of the locking mechanism of FIG. 43, depicting the locking mechanism in a locked state and a retention member of the locking mechanism depicted in cross-section.

FIG. 44B is a detail side view of the locking mechanism of FIG. 43, depicting the locking mechanism in an unlocked state and the retention member of the locking mechanism depicted in cross-section.

FIG. 45A is a partial perspective view of a delivery assembly comprising the frame of FIG. 12 and a delivery apparatus according to one example, depicting the frame in a radially-compressed state.

FIG. 45B is a partial perspective view of the delivery assembly of FIG. 45A, depicting the frame in a radially-expanded state.

FIG. 46A is a cross-sectional view of the delivery assembly of FIG. 45A, depicting the delivery apparatus uncoupled from a proximal end portion of a frame.

FIG. 46B is a cross-sectional view of the delivery assembly of FIG. 45A, depicting the delivery apparatus coupled to a distal end portion of the frame and in a released state.

FIG. 46C is a cross-sectional view of the delivery assembly of FIG. 45A, depicting the delivery apparatus coupled to the distal end portion of the frame and in a locked state.

FIG. 46D is a cross-sectional view of the delivery assembly of FIG. 45A, depicting the delivery apparatus uncoupling from to the distal end portion of the frame.

FIG. 46E is a cross-sectional view of the delivery assembly of FIG. 45A, depicting the delivery apparatus further uncoupling from to the distal end portion of the frame.

FIG. 47 is a cross-sectional view of the delivery assembly of FIG. 45A, depicting the delivery apparatus coupled to a distal end portion of the frame and in an alternative released state.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

### Introduction to the Disclosed Technology

The mechanically-expandable prosthetic heart valves disclosed herein can be radially compressed and/or expanded, as well as locked in place, by an expansion and locking mechanism. As one example, the prosthetic heart valves can be crimped on or retained by a delivery apparatus in a radially-compressed state during delivery and then radially expanded (and axially shortened) to a radially-expanded state once the prosthetic heart valve reaches the implantation site (or a location adjacent an implantation site). The expansion and locking mechanism may be configured to hold the prosthetic valve in the radially expanded state to prevent the valve from re-compressing after expansion and/or the prosthetic valve is released from the delivery apparatus.

This disclosure describes actuation and/or locking mechanisms that are integrally formed with the frame of the prosthetic heart valve and/or are coupled to the frame so as to be radially aligned with the struts of the frame. This can, for example, help reduce the radial profile of the prosthetic valve in the radially-compressed state and/or prevent the actuation/locking mechanisms from interfering with the valve structure (e.g., the leaflets) during operation of the prosthetic valve. Related delivery apparatus and methods of using the disclosed prosthetic heart valves and delivery apparatus are also described herein. The disclosed prosthetic heart valves, delivery apparatus, and methods can, for example, allow the prosthetic heart valve to be actuated (e.g., radially expanded and/or compress) and locked in a desired configuration. The disclosed actuation and/or locking mechanisms are relatively easier to manufacturer and/or assemble, are more robust, and/or easier to use than typical actuation and locking mechanisms. This can, among other things, help to ensure that help to ensure that the mechanically-expandable prosthetic heart valve is safely and securely implanted within a patient and continues to function properly after the implantation procedure. Additionally (or alternatively), the disclosed delivery apparatus and related methods can, for example, provide a relatively quick and easy way of coupling/releasing the prosthetic heart valve to/from the delivery apparatus. This can, for example, reduce the risk of mistakes and/or reduce the time it takes to implant a prosthetic heart valve.

The prosthetic heart valves, delivery apparatus, and methods disclosed herein may be described in relation to a particular implantation location (e.g., a native aortic valve) and/or using a particular delivery procedure (e.g., transfemoral delivery). These implantation locations and delivery procedures are merely examples. The disclosed devices and methods can be adapted to various other implantation locations (e.g., a native mitral valve, tricuspid valve, and/or pulmonary valve) and/or other delivery procedures (e.g., transapical, transseptal, etc.).

### Examples of the Disclosed Technology

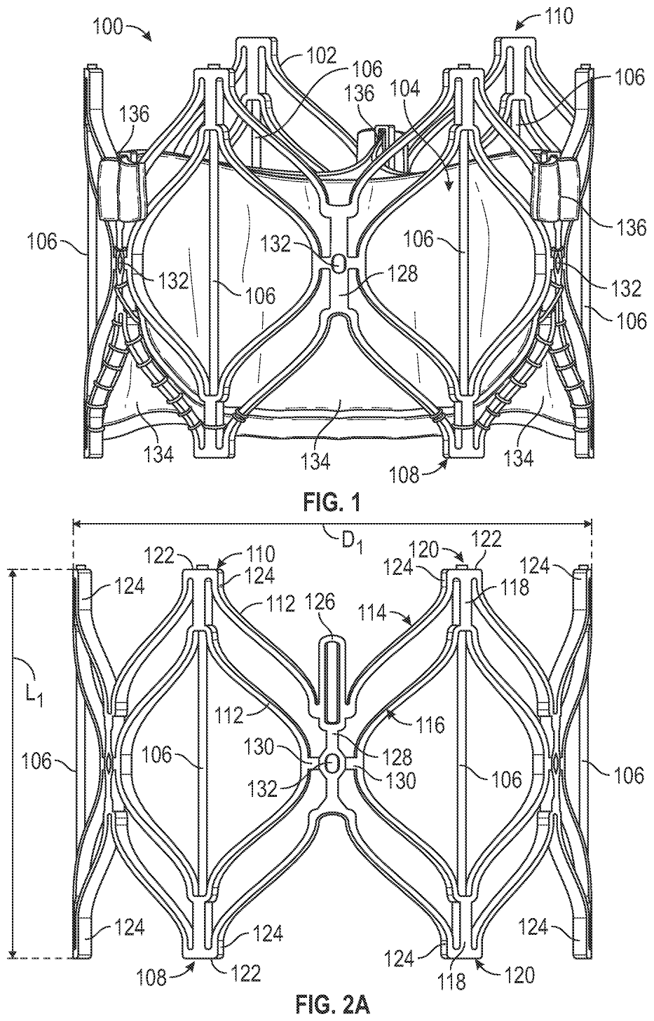

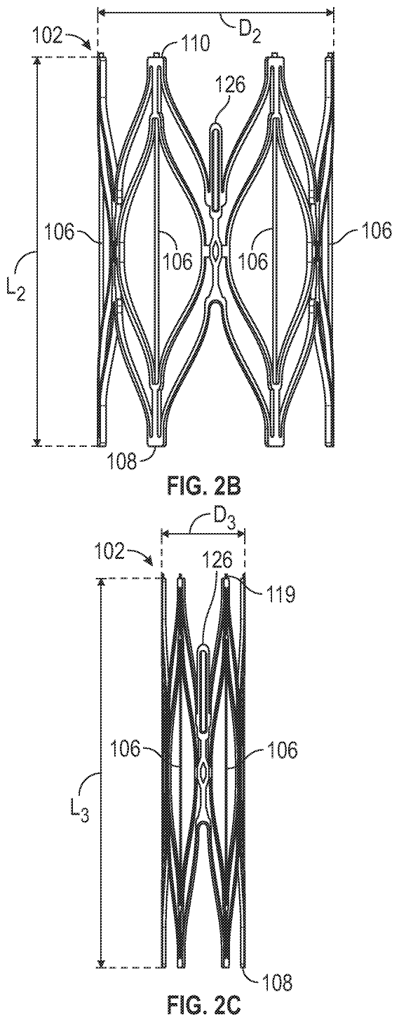

FIG. 1 depicts a mechanically-expandable prosthetic heart valve 100, according to one example. The mechanically-expandable prosthetic heart valve 100 (also referred to herein as “the prosthetic valve 100”) comprises three main components: a frame 102, a valve structure 104, and a plurality of actuators 106 (e.g., six actuators in the illustrated example). The frame 102 (which can also be referred to as “a stent” or “a support structure”) can be configured for supporting the valve structure 104 and for securing the prosthetic valve 100 within a native heart valve and/or within another support structure (e.g., an anchoring frame (such as a coil) and/or a previously implanted prosthetic valve (i.e., in a valve-in-valve procedure)). The valve structure 104 is coupled to the frame 102 (e.g., directly and/or indirectly via other components such a sealing skirt). The valve structure 104 is configured to allow blood flow through the prosthetic valve 100 in one direction (i.e., antegrade—the normal blood flow direction) and to restrict blood flow through the prosthetic valve 100 in the opposition direction (i.e., retrograde-opposite the normal blood flow direction). The actuators 106 are coupled to the frame 102 and are configured to adjust expansion of the frame 102 to a plurality of configurations including one or more functional or expanded configurations (e.g., FIG. 1), one or more delivery or compressed configurations (e.g., FIG. 2C), and/or one or more intermediate configurations between the functional and delivery configurations (e.g., FIG. 2B). It should be noted that the valve structure 104 of the prosthetic valve 100 is not shown FIGS. 2A-2C to better illustrate other components of the prosthetic valve 100.

Referring now to FIGS. 1-2A, the frame 102 of the prosthetic valve 100 has a first end 108 and a second end 110. In the depicted orientation, the first end 108 of the frame 102 is an inflow end and the second end 110 of the frame 102 is an outflow end. In other examples, the first end 108 of the frame 102 can be the outflow end and the second end 110 of the frame 102 can be the inflow end.

The frame 102 includes a plurality of struts 112, which are interconnected. In some examples, the struts can define a plurality of cells. For example, in the illustrated example, the struts 112 define a row of six primary cells. The frame 102 also comprises a row of six secondary cells, which are each nested with a respective primary cell. Accordingly, the primary cells and the secondary cells can also be referred to as “outer cells” and “inner cells,” respectively. The primary cells 114 and the secondary cells 116 are interconnected at their ends by vertical struts 118 (which can also be referred to as “non-pivoting struts”). The primary cells 114 and the secondary cells 116 each comprise a tear-drop like shape, which also resembles a hexagonal shape but with curved side. Portions of the primary cells and the secondary cells can also be described as having a “wishbone” shape. As such, the primary and secondary cells of the frame may be described as forming a “parallel wishbone” or “double wishbone” configuration. The primary and/or secondary cells can comprise various other shapes in other examples.

The primary cells 114 and/or the vertical struts 118 of the frame 102 form apices 120 at the first end 108 and the second end 110 of the frame 102. In the depicted example, each apex 120 comprises a “T” shape defined by a respective vertical strut 118 and a pair of angled struts of a primary cell 114. Each apex 120 comprises a flat (or at least substantially flat) end surface 122 extending between two vertically-oriented side surfaces 124. In other examples, the apices of the frame can comprise various other shapes (e.g., rounded).

The frame can further comprise a plurality of leaflet attachment structures. For example, as depicted in FIG. 2A, the frame 102 comprises a plurality of commissure windows 126 disposed circumferentially between adjacent pairs of the primary cells 114 of the frame. The commissure windows 126 are spaced axially from the apices 120 at the second end 110 of the frame 102 toward the first end 108 of the frame. In other examples, the commissure windows can be disposed at various other axial positions relative to the apices of the frame (e.g., axially aligned with the apices at the second end of the frame). In the depicted example, the commissure windows 126 comprise an opening that is bounded on all sides in a “closed” configuration. In other examples, the commissure windows can comprise an “open” configuration (e.g., a U-shaped slot). The commissure windows 126 extend from vertical struts 128 in a cantilevered manner. In other examples, the commissure windows 126 can be supported at multiple locations in a non-cantilevered manner.

The frame 102 can comprise various other struts and/or openings. For example, the frame 102 comprises a pair of circumferentially-extending struts 130 (which may also be referred to as “laterally-extending struts”) extending from the vertical struts 128 of the secondary cells 116. The frame 102 also comprises apertures 132 disposed in the vertical struts 128.

The struts 112 of the frame 102 are configured such that the frame 102 can move between a plurality of radial configurations. For example, FIG. 2A depicts a radially-compressed configuration, FIG. 2B depicts a partially radially-expanded configuration, and FIG. 2C depicts a radially-compressed configuration. The depicted configurations are exemplary, and the frame can be expanded or compressed to a lesser or greater extent than depicted. As the frame moves between the various configurations, some of the struts of the frame deflect or pivot relative to each other. For example, the angled struts (which can also be referred to as “diagonal struts”) (i.e., the non-vertically and non-horizontally oriented struts) deflect relative to the vertically and horizontally oriented struts. In this manner, the frame of the prosthetic valve axially elongates when the frame is radially compressed and axially foreshortens when the frame is radially expanded. Thus, a diameter D1 of the prosthetic valve 100 in the radially-expanded state (FIG. 2A) is greater than a diameter D2 of the prosthetic valve 100 in the partially radially-expanded state (FIG. 2B) and a diameter D3 of the prosthetic valve 100 in the radially-compressed state (FIG. 2C), and the diameter D2 of the prosthetic valve 100 in the partially radially-expanded state is greater than the diameter D3 of the prosthetic valve 100 in the radially-compressed state (i.e., D1>D2>D3). Oppositely, a length L1 of the prosthetic valve 100 in the radially-expanded state is less than a length L2 of the prosthetic valve 100 in the partially radially-expanded state and a length L3 of the prosthetic valve 100 in the radially-compressed state, and the length L2 of the prosthetic valve 100 in the partially radially-expanded state is less than the length L3 of the prosthetic valve 100 in the radially-compressed state (i.e., L1<L2<L3).

To facilitate movement between the expanded and compressed configurations, the frame can be formed of a deformable material, including biocompatible metals and/or biocompatible polymers. Exemplary biocompatible metals from which the frame can be formed include stainless steel, cobalt chromium alloy, and/or nickel titanium alloy (which can also be referred to as “NiTi” or “nitinol”).

The frame can be formed of a shape memory material (e.g., nitinol) such that the frame can be shape-set to a particular configuration and then elastically deformed to one or more other configurations. For example, the frame 102 is formed of nitinol and shape-set in the partially radially-expanded configuration (e.g., FIG. 2B). The frame 102 can be elastically deformed to the radially-compressed configuration (e.g., FIG. 2C) and to the radially-expanded configuration (e.g., FIG. 2A). The frame can be elastically deformed to the depicted configuration and/or various other configurations, for example, by using the actuators 106, a delivery apparatus, and/or a crimping device), as further described below.

In other examples, the frame can be formed of a plastically-deformable material (e.g., stainless steel or cobalt chromium alloy) such that the frame can be formed in a particularly configuration and then plastically deformed to one or more configurations which are radially smaller or larger than the configuration in which the frame is formed.

The frame 102 is formed from a single piece of material (e.g., a metal tube). This can be accomplished, for example, via laser cutting, electroforming, and/or physical vapor deposition. In other examples, the frame can be constructed by forming individual components coupling the individual components together (e.g., via welding, brazing, and/or other means for bonding).

Referring again to FIG. 1, the valve structure 104 of the prosthetic valve 100 is coupled to the frame 102. The valve structure 104 is configured to allow blood flow through the prosthetic valve 100 from the inflow end 108 to the outflow end 110 in an antegrade direction and to restrict blood from through the prosthetic valve 100 from the outflow end 110 to the inflow end 108 in a retrograde direction. The valve structure can include various components including a leaflet assembly comprising one or more leaflets. For example, the valve structure 104 has a leaflet assembly with three leaflets 134.

The leaflets 134 of the prosthetic valve 100 can be made of a flexible material. For example, the leaflets 134 can be made from in whole or part, biological material, bio-compatible synthetic materials, or other such materials. Suitable biological material can include, for example, bovine pericardium, equine pericardium, porcine pericardium, and/or pericardium from other sources.

The leaflets 134 can be arranged to form commissures 136 (e.g., pairs of adjacent leaflets), which can, for example, be mounted to the frame at the commissure windows 126 (e.g., via sutures, fabric, adhesive, and/or other means for mounting). Further details regarding prosthetic heart valves, including the manner in which the valve structure 104 can be coupled to the frame 102 of the prosthetic valve 100, can be found in U.S. Pat. Nos. 6,730,118, 7,393,360, 7,510,575, 7,993,394, and 8,652,202, and U.S. Publication No. 2018/0325665, which are incorporated by reference herein.

Referring again to FIGS. 1-2A, the actuators 106 of the prosthetic valve 100 are mounted to and spaced circumferentially around the frame 102. In the illustrated example, the prosthetic valve 100 comprises six actuators 106. In other examples, the prosthetic valve can comprise fewer or more than six actuators (e.g., 1-5 or 7-15). The actuators 106 are configured to, among other things, radially expand and/or radially compress the frame 102. For this reason, the actuators 106 can also be referred to as “expansion mechanisms.”

The actuators can be formed of various materials. For example, in some instances, the actuators can be a rod or shaft. In such instances, the actuators can be formed as separated components from the frame, which are then coupled thereto (e.g., via welding, adhesive, fasteners, or other means for coupling). Alternatively, the actuators and the frame can be integrally formed as a unitary structure (e.g., by forming the frame and actuators from a tube). In other instances, the actuators can be a cable, wire, cord, suture, or other relatively flexible material (i.e., compared to a shaft or rod). In such instances, the flexible actuator can be coupled to the frame by tying or looping the actuators around the struts of the frame and/or by coupling the actuator to the frame via a fastener (e.g., a grommet), adhesive, and/or other means for coupling.

In some examples, the actuators are configured for rotational actuation. For example, an actuator can comprise external threads along one or more portions of the actuator (e.g., similar to a bolt or screw). A first end portion of the actuator can be coupled to a first portion (e.g., an inflow end portion) of the frame (e.g., via the head of the screw) such that the actuator can rotate relative to the first portion of the frame but is axially fixed thereto. A second end portion of the actuator can extend through a lumen of the frame disposed at another location (e.g., an outflow end portion) of the frame. The lumen of the frame can comprise corresponding internal threads configured to mate with the external threads of the actuator. In this manner, rotating the actuator in a first direction (e.g., clockwise) relative to the frame results in radial expansion of the frame as the first end portion of the frame and the second end portion of the frame move axially toward each other along the threads of the actuator. Likewise, rotating the actuator in a second direction (e.g., counterclockwise) relative to the frame results in radial compression of the frame as the first end portion of the frame and the second end portion of the frame move axially away from each other along the threads of the actuator. Due to the threaded engagement between the frame and the actuators, the actuators lock the frame at a desired expanded configuration when the actuators are stationary relative to the frame. Accordingly, such actuators can also be referred to as “lockers,” “locking members,” or “locking mechanisms.” Such rotational actuators, however, have their shortcomings. For example, forming the actuators and frame, which are very small components, with threads can present manufacturing and reliability challenges.

Accordingly, in other examples, the actuators are configured for linear actuation. In such instances, the actuators 106 comprise fixed end portions fixedly coupled to one portion of the frame (e.g., the first end portion) and free end portions movably coupled to another portion of the frame (e.g., the second end portion). For example, the fixed end portions of the actuators 106 can be coupled to and/or extend axially from the vertical struts 118 at the inflow end portion of the frame 102, across the primary and second cells and through a lumen traversing the vertical struts 118 at the outflow end portion of the frame 102. The actuator 106 can be used to expand the frame 102 by pulling the actuator 106 toward the outflow end portion of the frame while applying an opposing force on the apices of the outflow end portion of the frame (e.g., with a delivery apparatus). These axially-opposing forces together apply a compressive force to the frame and result in radial expansion of the frame. The frame can be radially compressed by reducing tension on the actuators and allowing the elastic properties of the frame to radially compress the frame to its neutral or resting state and/or by an external radially inward force (e.g., a crimping device and/or native anatomy within a patient's body). Frames with linear actuation provide one or more advantages, including improved manufacturability and reliability relative to frames with rotational actuation. Despite these advantages, frames with linear actuation require a locking mechanism to retain the actuation member's position relative to the frame in order to secure the frame in one or more desired configurations. Several examples of actuators and locking mechanisms are described further below.

Each of the actuators can be configured to form a releasable connection with one or more respective actuation shafts of a delivery apparatus. This releasable connection can, for example, include a threaded connection, a plurality of interlocking shafts, and other means of forming a releasable connection. Several examples of releasable connections between the actuators and a delivery apparatus are described below.

The prosthetic valves described herein can also comprise one or more optional components. For example, in some examples, a prosthetic valve can include one or more sealing skirts. For example, the prosthetic valve 100 can include an inner skirt mounted on the inner surface of the frame 102. The inner skirt can function as a sealing member to prevent or decrease paravalvular leakage, to anchor the leaflets to the frame, and/or to protect the leaflets 134 against damage caused by contact with the frame 102 during crimping and during operation of the prosthetic valve 100 (i.e., the opening and closing of the leaflets). The prosthetic valve 100 can also include an outer skirt mounted on the outer surface of the frame 102. The outer skirt can function as a sealing member for the prosthetic valve by sealing against the tissue of the native valve annulus and thus reducing paravalvular leakage around the prosthetic valve. The inner and outer skirts can be formed from any of various suitable biocompatible materials, including any of various synthetic materials (e.g., PET) and/or natural tissue (e.g., pericardial tissue). The inner and outer skirts can be mounted to the frame using sutures, adhesive, and/or other means for attaching the skirts to the frame.

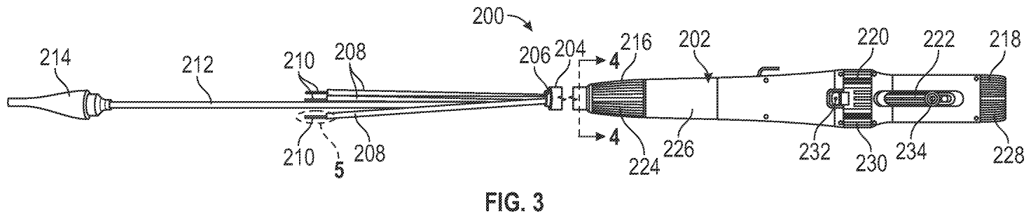

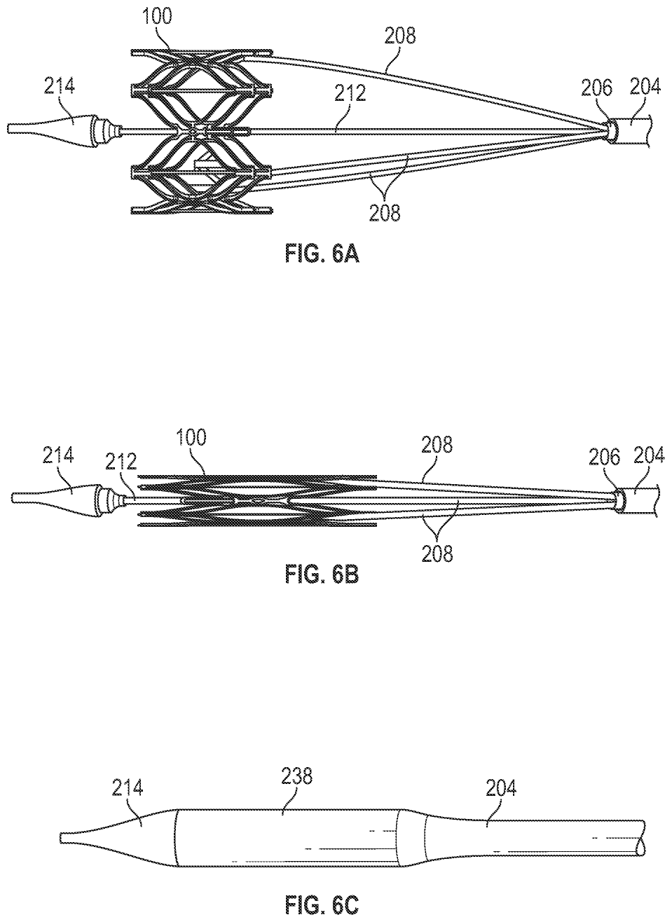

FIGS. 3-5 depict a delivery apparatus 200 and its components, according to one example. The delivery apparatus 200 can also be referred to as a “valve catheter” or a “delivery catheter.” The delivery apparatus 200 comprises a handle 202, a first shaft 204, a second shaft 206, three support sleeves 208, three actuation shafts 210, a nosecone shaft 212, and a nosecone 214. The handle 202 is configured for manipulating the various shafts and/or sleeves relative to each other. A prosthetic valve (e.g., the prosthetic valve 100) can be releasably coupled to the distal end portion of the delivery apparatus 200 (see, e.g., FIGS. 6A-6B), and the delivery apparatus 200 can be used for positioning the prosthetic valve 100, and/or for expanding, compressing, and locking the prosthetic valve 100 in a desired radially expanded configuration.

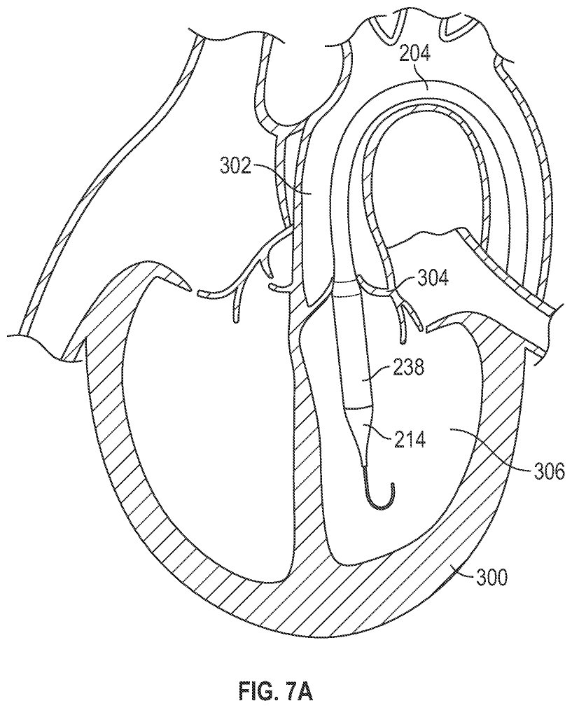

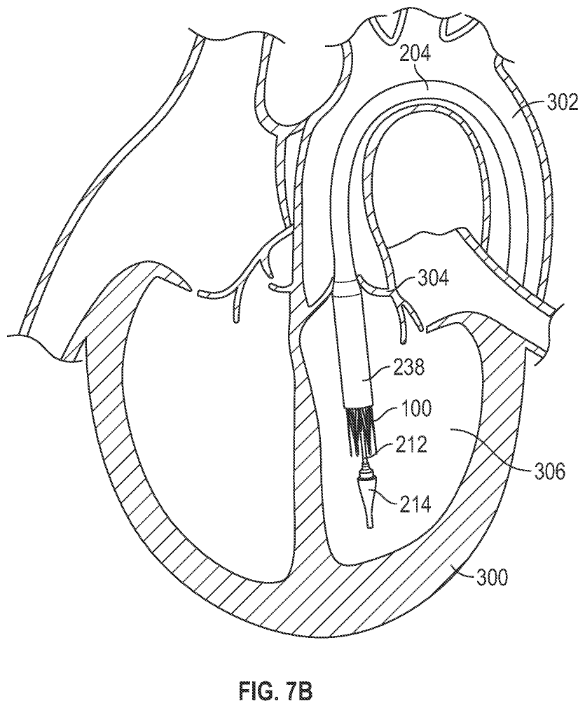

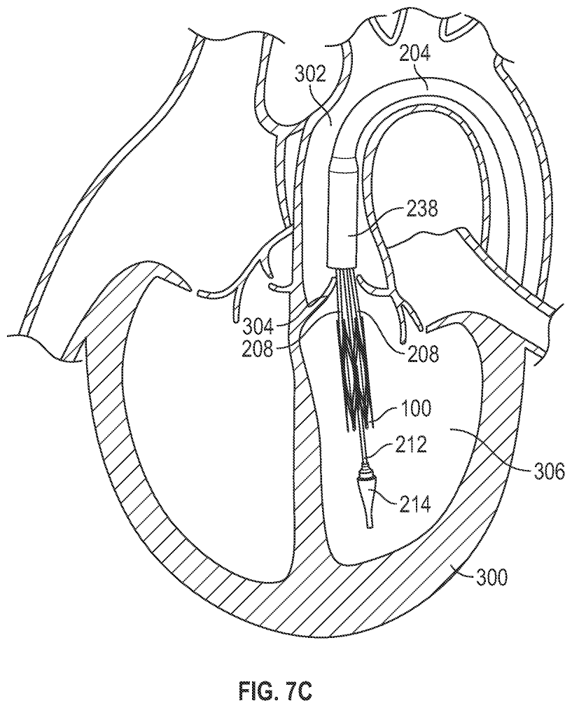

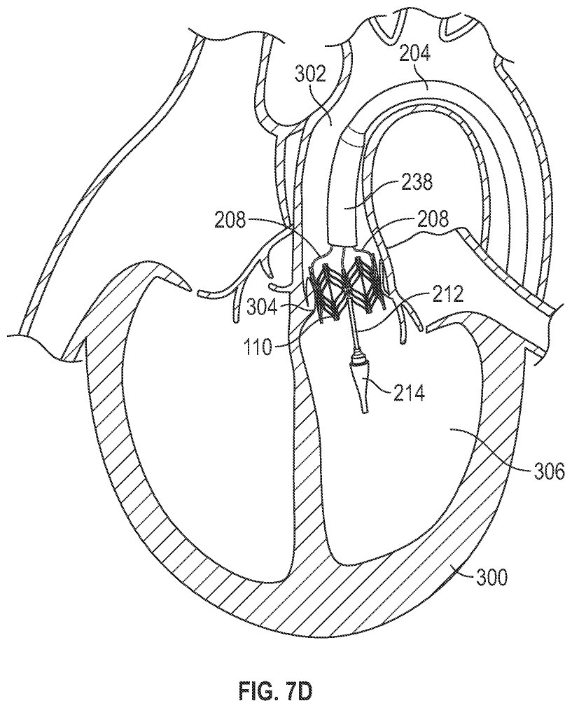

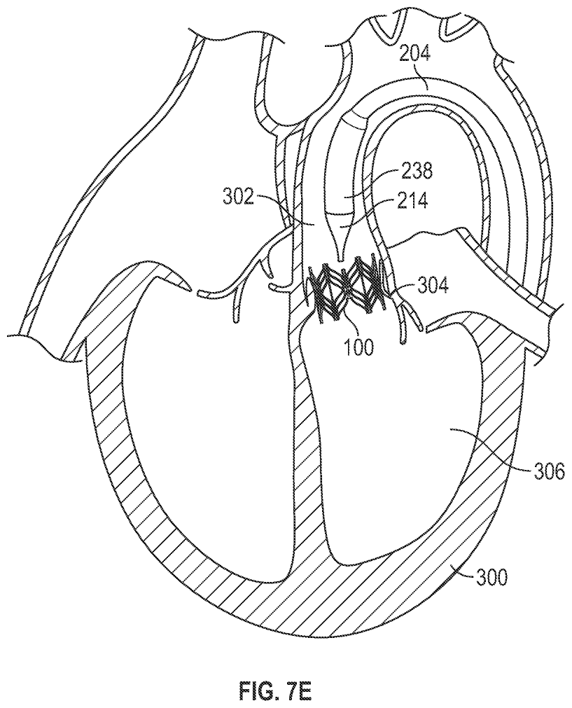

In the illustrated example, the delivery apparatus 200 comprises three pairs of support sleeves 208 and actuation shafts 210. In other examples, the delivery apparatus 200 can comprise less than three (e.g., 1-2, including 1 or 2) or more than three (e.g., 4-15, including 6-12, 6-9, 4, 5, 6, 7, 8, 9, 10, 11, 12, 13, 14, or 15) pairs of support sleeves 208 and actuation shafts 210, depending on the number of actuators a prosthetic valve includes. For example, although only three pairs of support sleeves 208 and actuation shafts 210 are depicted in FIGS. 6A-7E for purposes of illustration, the delivery apparatus 200 can comprise six pairs of support sleeves and actuation shafts when using the delivery apparatus 200 to implant the prosthetic valve 100 since the prosthetic valve 100 comprises six actuators.

The handle 202 of the delivery apparatus 200 can comprise one or more mechanisms configured to move the shafts and sleeves relative to each other. For example, as depicted in FIG. 3, the handle 202 comprises a deployment mechanism 216, an actuation mechanism 218, a release mechanism 220, and/or a nosecone positioning mechanism 222.

The deployment mechanism 216 of the handle 202 is coupled to the first shaft 204 and the second shaft 206 and is configured to move the first shaft 204 and the second shaft 206 axially relative to each other. As further explained below, the first mechanism 216 of the handle 202 can be used to deploy a prosthetic valve from a delivery capsule (or “a delivery sheath”) (e.g., a distal end portion) of the first shaft 204 (see FIGS. 7A-7C).

In the illustrated example, the deployment mechanism 216 includes a first knob 224 configured for actuating the deployment mechanism 216. In other examples, the deployment mechanism 216 can comprise various other types of actuators configured for actuating the deployment mechanism 216, such as buttons, switches, etc. The deployment mechanism 216 can also include one or more other components (such as electric motors, rotatable shafts, drive screws, gear assemblies, etc.) configured to facilitate and/or restrict relative axial movement between the first shaft 204 and the second shaft 206. For example, the deployment mechanism 216 can be configured such that rotating the first knob 224 (and/or an electric motor) relative to a housing 226 of the handle 202 results in relative axial movement between the first shaft 204 and the second shaft 206.

The actuation mechanism 218 of the handle 202 is coupled to the actuation shafts 210 and is configured to move the actuation shafts 210 axially relative to the support sleeves 208. When a prosthetic valve is coupled to the delivery apparatus 200 via the actuation shafts 210, the actuation mechanism 218 can be used to radially expand and/or compress the prosthetic valve, as further explained below. Accordingly, the actuation mechanism 218 can also be referred to as “an expansion mechanism.”

In the illustrated example, the actuation mechanism 218 comprises a second knob 228 configured for actuating the actuation mechanism 218. In other examples, the actuation mechanism 218 can comprise various other types of actuators. The actuation mechanism 218 can also include one or more additional components configured to facilitate and/or restrict relative axial movement of the actuation shafts 210 relative to the support sleeves 208. For example, the actuation mechanism 218 can comprise electric motors, drive screws, gear assemblies, and/or other components. In some examples, the actuation mechanism 218 can be configured such that rotating the second knob 228 (and/or an electric motor) relative to the housing 226 of the handle 202 results in relative axial movement between the actuation shafts 210 and the support sleeves 208.

The release mechanism 220 of the handle 202 is also coupled to the actuation shafts 210 and is configured to rotate the actuation shafts 210 relative to the support sleeves 208. In this manner, the release mechanism 220 can be used to simultaneously couple/release each of the actuation shafts 210 to/from the prosthetic valve 100, as further described below. Thus, the release mechanism 220 can also be referred to as “a coupling mechanism.”

In the illustrated example, the release mechanism 220 comprises a third knob 230 configured for actuating the release mechanism 220. In other examples, the release mechanism 220 can comprise various other types of actuators. The release mechanism 220 can also comprise one or more other components (e.g., a gear assembly and/or an electric motor) configured to facilitate and/or restrict relative rotational movement between the actuation shafts 210 and the support sleeves 208. For example, the release mechanism 220 can be configured such that rotating the third knob 230 relative to the housing 226 results in rotation of the actuation shafts 210 relative to the support sleeves 208. The release mechanism 220 can also comprise a lock mechanism, such as a switch 232 configured to selectively restrict rotation of the third knob 230 relative to the housing 226. In this manner, the switch 232 can prevent or reduce the likelihood that the delivery apparatus 200 is inadvertently released from a prosthetic valve.

The nosecone positioning mechanism 222 of the handle 202 is coupled to the nosecone shaft 212 and is configured to move the nosecone shaft 212 and the nosecone 214 axially relative to the first shaft 204 and the second shaft 206.

In the illustrated example, the nosecone positioning mechanism 222 comprises a slider 234 configured for actuating the nosecone positioning mechanism 222. The nosecone positioning mechanism 222 can comprise various other components configured to facilitate and/or restrict relative axial movement of the nosecone shaft 212, the first shaft 204, and the second shaft 206. For example, in some examples, the nosecone positioning mechanism 222 can comprise one or more biasing members (e.g., springs) configured to bias the nosecone shaft 212 to a pre-determined axial position relative to the first shaft 204 and the second shaft 206. In such instances, the slider 234 can be biased to a particular axial position relative to the housing 226 (e.g., to a proximal position). The nosecone shaft 212 can be moved axially relative to the first and second shafts by sliding the slider 234 relative to the housing 226 with sufficient force to overcome the opposing force of the biasing members. Upon release, the slider 234 can return to the biased position. In other examples, the nosecone positioning mechanism can comprise a rotatable knob, an electric motor, and/or a drive screw configured to convert relative rotational movement between the knob (and/or motor) and the housing into relative axial movement between the nosecone shaft and the first and second shafts.

Referring now to FIGS. 3-4, a proximal end portion of the first shaft 204 is coupled to and extends distally from the handle 202. The first shaft 204 comprises a lumen 236 for housing the second shaft 206 of the delivery apparatus 200. The distal end portion of the first shaft 204 comprises a delivery capsule 238 (which can also be referred to as “a sheath”) configured to receive a prosthetic valve in the radially-compressed configuration (see FIGS. 7A-7B). Alternatively, the delivery capsule can be a separately formed component that is coupled to the distal end portion of the first shaft 204.

As depicted in FIG. 3, the second shaft 206 extends coaxially through and is axially movable relative to the first shaft 204. The second shaft 206 can comprise a plurality of lumens extending axially therethrough and can thus be referred to as “a multi-lumen shaft.” For example, as shown in FIG. 4, the second shaft 206 includes three actuation lumens 240 spaced circumferentially relative to each other. The actuation lumens 240 can be configured to receive respective actuation shafts 210 and/or support sleeves 208. In the illustrated example, the actuation lumens 240 are evenly spaced relative to each other (e.g., spaced apart by about 120 degrees). In other examples, the actuation lumens 240 can be non-evenly spaced relative to each other. The second shaft 206 can in other examples include less (e.g., 1-2, including 1 or 2) or more (e.g., 4-15, including 6-12, 6-9, 4, 5, 6, 7, 8, 9, 10, 11, 12, 13, 14, or 15) actuation lumens.

Referring still to FIG. 4, the second shaft 206 also includes a guidewire lumen 242. The guidewire lumen 242 can be radially centrally disposed in the second shaft 206.

In some examples, the second shaft 206 optionally include one or more additional lumens. For example, the second shaft 206 can comprise a recompression lumen configured to receive one or more shaft, sutures, etc. that can be used to recompress a prosthetic valve. The recompression lumen can be disposed radially outwardly relative to the guidewire lumen 242. In some examples, the recompression lumen can be radially aligned with and/or spaced circumferentially relative to the actuation lumens 240.

The support sleeves 208 can extend distally from respective actuation lumens 240 of the second shaft 206 and can be configured to contact apices of the prosthetic valve. The support sleeves 208 can be relatively more rigid than the actuation shafts 210. As such, the support sleeves 208 can be used to apply distally-directed forces the prosthetic valve, which can oppose proximally-directed forces applied to prosthetic valve by the actuation shafts 210, thereby enabling expansion of the prosthetic valve caused by relative axial movement between the actuators and the frame of the prosthetic valve.

In the illustrated example, the support sleeves 208 are relative short tubes that are coupled to the distal end portion of the second shaft 206 but do not extend all the way through the second shaft 206 to the handle 202. The support sleeves 208 can, in some instances, be secured to the inner surfaces of the second shaft 206 that define the actuation lumens 240 (e.g., via adhesive). In other examples, proximal end portions of the support sleeves 208 can be coupled to the handle 202, and the support sleeves 208 can extend through respective actuation lumens 240 of the second shaft 206 and beyond the distal end of the second shaft 206. In either case, each of the support sleeves 208 comprises a lumen configured to receive a respective actuation shaft 210, as shown in FIG. 4.

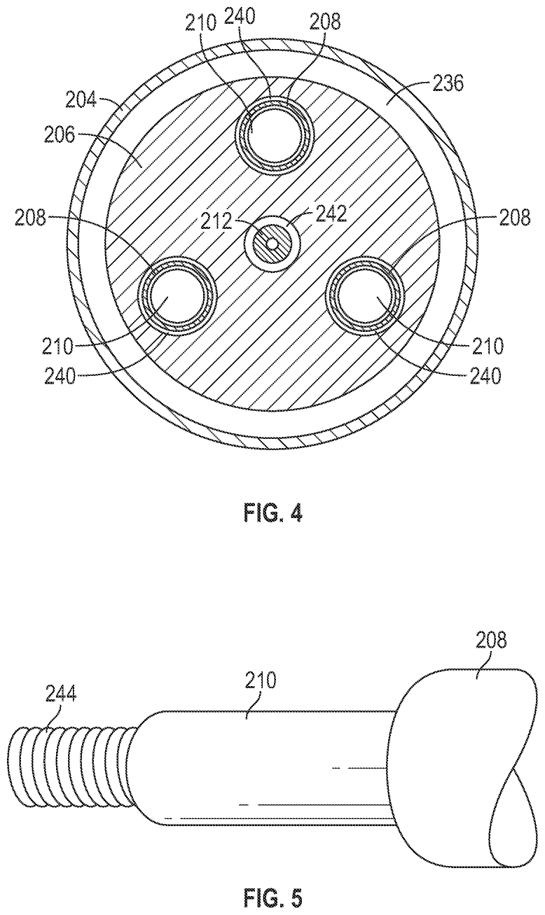

The actuation shafts 210 can extend distally from the handle 202, through respective actuation lumens 240 of the second shaft 206, and through the lumens of respective support sleeves 208. The distal end portions of the actuation shafts 210 can comprise mating features configured to releasably couple the actuation shafts to the actuators of the prosthetic valve. For example, as shown in FIG. 5, the distal end portions of the actuation shafts 210 comprise external threads 244 configured to mate with corresponding internal threads of the actuators of the prosthetic valve.

In some examples, the actuation shafts 210 can be relatively flexible members. For example, the actuation shafts can be wires, cables, cords, sutures, etc. In other examples, the actuation shafts can be relatively rigid members, such as a rod. In other examples, the actuation shafts 210 can comprise one or more relatively flexible segments (e.g., at the distal end portions) and one or more relatively rigid segments (e.g., at the proximal end portions).

The optional recompression shaft extends from the handle through the recompression lumen of the second shaft. The recompression shaft comprises a lumen through which a recompression member (e.g., wire, cable, suture, etc.) extends. The recompression member can extend around the prosthetic valve in a lasso-like manner. As such, the recompression member can be used to aid recompression of the prosthetic valve by tensioning and thus constricting the recompression member around the prosthetic valve.

Referring now to FIG. 6A, the prosthetic valve 100 can be coupled to a distal end portion of the delivery apparatus 200 to form a delivery assembly, and the delivery apparatus 200 can be used to implant the prosthetic valve 100 within a patient's body (see FIGS. 7A-7E). The prosthetic valve 100 can be coupled to the delivery apparatus 200 by positioning the delivery apparatus 200 in the configuration shown in FIG. 6A. With the prosthetic valve 100 in the radially expanded configuration, the prosthetic valve 100 can be positioned over a proximal portion of the nosecone 214 and the nosecone shaft 212. The actuators 106 of the prosthetic valve 100 can be positioned adjacent the distal ends of the actuation shafts 210. The actuation shafts 210 can then be threadably coupled to the actuators 106.

With the prosthetic valve 100 releasably coupled to the delivery apparatus 200, the prosthetic valve 100 can be radially compressed by actuating the actuators 106, by tensioning a recompression member, and/or by inserting the prosthetic valve 100 and delivery apparatus 200 into a crimping device. FIG. 6B shows the prosthetic valve 100 in a radially compressed configuration. The first shaft 204 of the delivery apparatus 200 can be advanced over the second shaft 206 of the delivery apparatus 200 and the prosthetic valve 100 such that the prosthetic valve 100 is disposed within the lumen of the first shaft 204 and the distal end of the first shaft 204 abuts the nosecone 214, as shown in FIG. 6C. This can be accomplished, for example, by actuating the deployment mechanism 216 of the handle 202.

The distal end portion of the delivery assembly can then be inserted into a patient's vasculature, and the prosthetic valve 100 can be advanced to an implantation location using the delivery apparatus 200. For example, FIGS. 7A-7E show an exemplary implantation procedure for implanting the prosthetic valve 100 within a patient's heart 300 using a transfemoral delivery procedure. In other examples, various other delivery procedures can be used, such as transventricular, transapical, transseptal, etc.

Referring to FIG. 7A, the distal end portion of the delivery assembly is inserted into a patient's vasculature (e.g., over a guidewire) such that the first shaft 204 extends through the patient's aorta 302 and such that the nosecone 214 extends through the patient's native aortic annulus 304 and into the left ventricle 306 of the patient's heart 300. Turning to FIG. 7B, the prosthetic valve 100 can be deployed from the first shaft 204 of the delivery apparatus 200 by actuating the deployment mechanism 216 of the handle 202, which moves the first shaft 204 of the delivery apparatus 200 proximally relative to the second shaft 206 of the delivery apparatus 200 (and/or moves the second shaft 206 distally relative to the first shaft 204). The first shaft 204 can be moved further proximally such that the support sleeves 208 are exposed from the distal end of the first shaft 204, as depicted in FIG. 7C.

Once exposed from the delivery capsule 238 of the delivery apparatus 200, the prosthetic valve 100 can, in some instances, self-expand from the radially-compressed, delivery configuration to an intermediate (or neutral) configuration, which can also be referred to as “a partially-expanded configuration” (see, e.g., FIG. 2B). In some examples, the partially-expanded configuration can correspond to the diameter to which the frame of the prosthetic valve is shape-set, such as when the frame is formed of a shape-memory material (e.g., nitinol).

As shown in FIG. 7D, the prosthetic valve 100 can be mechanically expanded from the partially-expanded configuration (or from the radially-compressed configuration) to one or more functional configurations. This can be accomplished, for example, by actuating the actuation mechanism 218 of the handle 202 such that the actuation shafts 210 of the delivery apparatus 200 and the actuators 106 of the prosthetic valve 100 (which are coupled to the actuation shafts 210) move proximally relative to the support sleeves 208 of the delivery apparatus 200 and the frame 102 of the prosthetic valve 100 (which contact the distal ends of the support sleeves 208). When the actuators 106 are configured for rotation actuation (e.g., jack screw type actuators), rotating the actuators 106 relative to the frame results in expansion or contraction of the prosthetic valve 100 depending on the direction of rotation. When the actuators are configured for linear actuation, moving the actuators 106 axially (e.g., proximally/distally) relative to the frame 102 results in expansion or contraction of the prosthetic valve 100 depending on the direction of movement. When the prosthetic valve 100 is desirably positioned and secured within the native aortic annulus 304, the actuators 106 can be secured relative to the frame 102 (e.g., by the threaded connection and/or a locking mechanism) to retain the prosthetic valve 100 in the expanded state.

If re-positioning or retrieval of the prosthetic valve is desired, the actuation mechanism 218 can be used to actuate the actuators 106 to radially compress the prosthetic valve 100. In lieu of or in addition to using the actuation mechanism 218, the prosthetic valve 100 can be recompressed and repositioned and/or retrieved using a recompression member and/or the delivery capsule 238 via the deployment mechanism 216. In some instances, the recompression member and/or the delivery capsule can radially compress the prosthetic valve to a diameter that is smaller than is possible using only the actuators 106.

Once expanded and secured, the prosthetic valve 100 can be released from the delivery apparatus 200, as shown in FIG. 7E. This can be accomplished by actuating the release mechanism 220 of the handle 202. This rotates the actuation shafts 210 of the delivery apparatus 200 relative to the actuators 106 of the prosthetic valve 100, thereby de-coupling the threads 244 of the actuation shafts 210 from the threads of the actuators 106. The actuation shafts 210, the support sleeves 208, the second shaft 206, and the nosecone shaft 212 can then be withdrawn into the first shaft 204 such that the nosecone 214 abuts the distal end of the delivery capsule 238, and the delivery apparatus 200 can be removed from the patient's body.

Additional details regarding the delivery apparatus 200 (including, for example, the recompression member and the deployment, actuation, release, and nosecone mechanisms of the handle 202) are provided in International Patent Application Nos. PCT/US2020/063104 and U.S. PCT/US2021/022467, both of which are incorporated by reference herein.

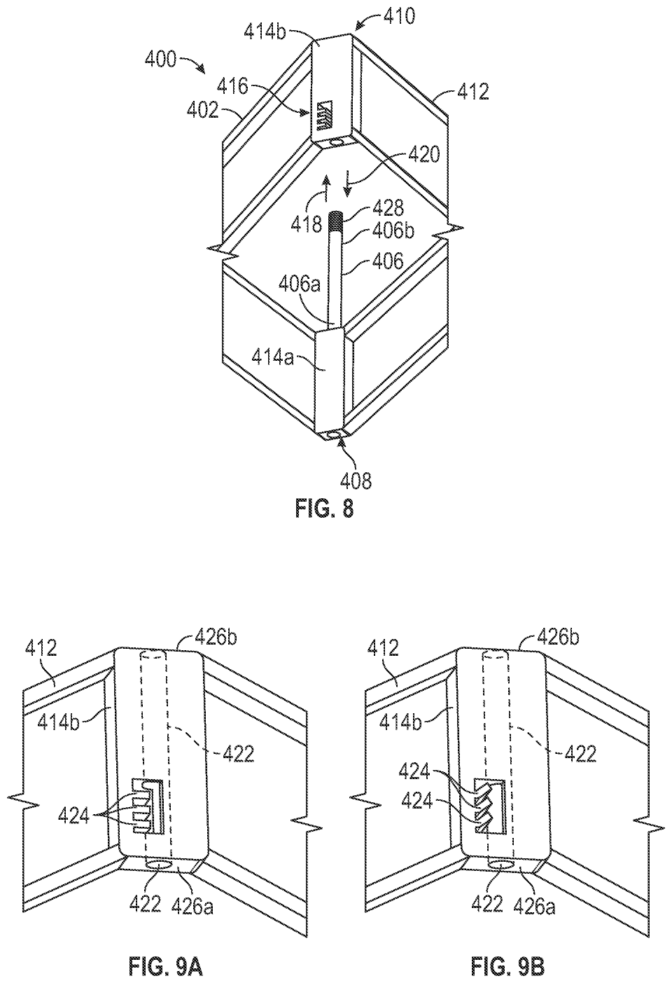

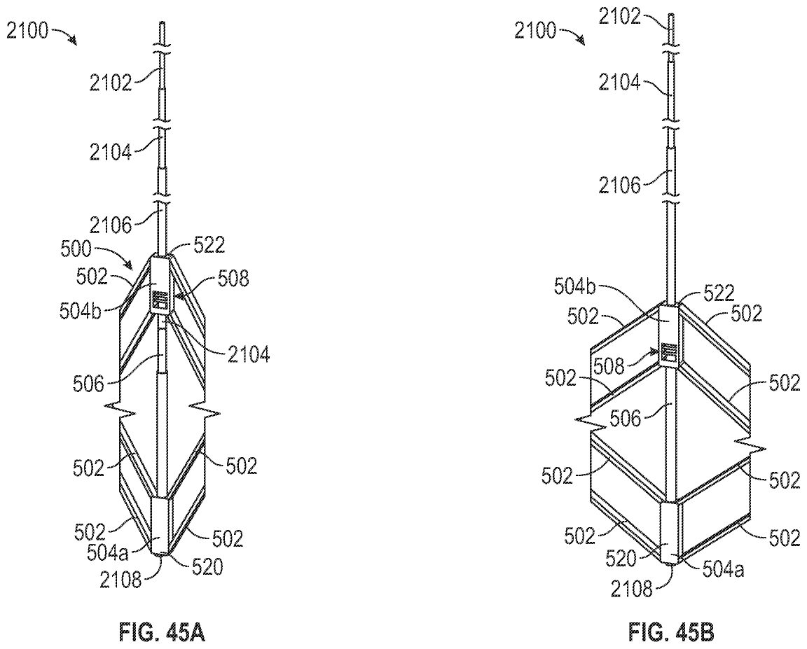

FIGS. 8-44B depict (in some instances schematically) portions of prosthetic heart valves and several examples of actuation members and locking mechanisms that can be used to expand/compress the prosthetic heart valve and/or to secure the prosthetic valve in one or more expanded configurations. FIGS. 45A-47 depict an exemplary delivery assembly comprising a prosthetic valve and a delivery apparatus, which are partially shown for purposes of illustration, as well as examples of how the delivery apparatus can be coupled to and released from the prosthetic valve.

FIGS. 8-11 depict a portion of a prosthetic heart valve 400. The prosthetic valve 400 can be configured generally similar to the prosthetic valve 100 in that the prosthetic valve 400 can include a frame 402, a valve structure, and one or more actuation members 406, which are similar in their purpose and/or function to the frame 102, the valve structure 104, and the actuators 106 of the prosthetic valve 100, respectively. The frame 402 is configured to support the valve structure and to anchor the prosthetic valve within a patient's vasculature (e.g., a native valve annulus), another prosthetic valve (e.g., for a valve-in-valve procedure), or a docking device (e.g., a stent or a coil). The valve structure is configured for regulating one-way blood flow through the valve from an inflow end 408 to an outflow end 410. The actuation members 406 (together with a delivery apparatus) are configured for moving the frame 402 from one or more radially-compressed configurations to one or more radially-expanded configurations, and vice versa.

The frame 402 of the prosthetic valve 400 is only partially shown in FIGS. 8-11. For example, only one region or segment is depicted. The frame 402 can include a plurality (e.g., 3-15) of regions that are substantially similar to the depicted region. For example, in some instances, the frame comprises six regions (which may also be referred to as “cells”). Referring to FIG. 8, the frame comprises a plurality of interconnected struts. The struts comprise a plurality of angled struts 412 extending between a plurality of vertically-oriented struts, including a first vertically-oriented strut 414a and a second vertically-oriented strut 414b (collectively or generically referred to as “the vertically-oriented struts 414”). The struts can define one or more cells (e.g., primary/outer and/or secondary/inner cells). The angled struts 412 are configured to deflect relative to the vertically-oriented struts 414 as the frame 402 expands and compresses radially. Accordingly, an angle between the angled struts 412 and the vertically-oriented struts 414 decreases when the frame 402 is radially compressed and increases when the frame 402 is radially expanded. This change in angle can be seen by comparing FIGS. 8 and 10.

The vertically-oriented struts 414 of the frame 402 can comprise various features and/or have one or more additional components coupled thereto. For example, as depicted in FIG. 8, the actuation member 406 is coupled to and extends from the first vertically-oriented strut 414a (which is located toward the inflow end 408), and the second vertically-oriented strut 414b (which is located toward the outflow end 410) comprises a locking mechanism 416. In other examples, the actuation member can be coupled to and extend from the second vertically-oriented strut 414b, and the first vertically-oriented strut 414a can comprise the locking mechanism. Additional vertically-oriented struts (i.e., in addition to the vertically-oriented struts 414) and/or other struts of the frame (e.g., the angled struts 412) can be configured for coupling the valve structure (and/or a sealing skirt) to the frame. For example, one or more other struts can comprise a window or slot formed therein configured to receive commissures of the valve structure. Such struts can be referred to as “commissure posts” or “commissure attachment posts.” In some examples, the commissure posts can be spaced axially from the outflow end 410 of the prosthetic valve 400 (e.g., in “valleys” between adjacent pairs of apices). In other examples, the commissure posts can be disposed at or adjacent apices of the prosthetic valve. Exemplary commissure posts and attachment of the valve structure are described above with reference to the prosthetic valve 100 and depicted in FIGS. 1 and 2A.

Returning to FIG. 8, the actuation member 406 and the locking mechanism 416 are integrally formed with the struts of the frame 402 as a single, unitary component. This can be accomplished, for example, by cutting the frame 402, the actuation member 406, and the locking mechanism 416 from a tubular piece of material (e.g., a metal tube). In such examples, the actuation member 406 and/or the locking mechanism 416 can be referred to as components of the frame 402. In other examples, one or more of the components (e.g., the frame and the actuation member) can be formed as separate components, and the separately-formed components can be coupled together (e.g., via welding, fasteners, adhesive, and/or other means for coupling).

The actuation member 406 can be a rod or shaft and comprises a fixed end portion 406a and a free end portion 406b. The fixed end portion 406a is coupled to the first vertically-oriented strut 414a, and the free end portion 406b extends toward the second vertically-oriented strut 414b and the locking mechanism 416. When the frame 402 of the prosthetic valve 400 is in a radially-compressed configuration (e.g., a delivery configuration), the free end portion 406b of the actuation member 406 is spaced apart from the locking mechanism 416. When the frame 402 of the prosthetic valve 400 radially expands, the free end portion 406b of the actuation member 406 moves toward the locking mechanism 416 (i.e., in a first direction depicted by the arrow 418). When the frame 402 of the prosthetic valve 400 radially compresses, the free end portion 406b of the actuation member 406 moves away from the locking mechanism 416 (i.e., in a second direction depicted by the arrow 420). The frame 402 can freely move between various radially expanded/compressed configurations so long as the free end portion 406b is disengaged from the locking mechanism 416. When the frame 402 is radially expanded to a predetermined diameter, the locking mechanism 416 engages the free end portion 406b of the actuation member 406 and prevents the actuation member 406 from separating from the locking mechanism 416, thereby preventing or restricting radial compression of the frame 402. This configuration can be referred to as “a locked configuration.” In the locked configuration, the locking mechanism 416 prevents the actuation member 406 from moving in the second direction (see arrow 420) relative to the locking mechanism 416. After initial engagement between the locking mechanism 416 and the actuation member 406, the locking mechanism 416 allows the actuation member 406 to move farther in the first direction (see arrow 418) relative to the locking mechanism 416, which allows further radial expansion of the frame 402. In other words, the locking mechanism 416 allows one-way movement of the actuation member 406.

Referring to FIG. 9A, the locking mechanism 416 comprises a channel 422 and one or more retention elements 424 (which can also be referred to as “retention tabs” or “tongues”). Although three retention elements 424 are depicted in the illustrated example, a locking mechanism can have fewer (e.g., 1-2) or more (e.g., 4-15) than three retention elements. The channel 422 extends axially from a first end 426a to a second end 426b of the second vertically-oriented strut 414b and is configured to receive the actuation member 406 and an actuation shaft of the delivery apparatus. The retention elements 424 extend laterally (which may also be referred to as “circumferentially”) and partially obstruct the channel 422. Accordingly, the retention elements 424 engage the actuation member 406 when the actuation member 406 is disposed in the channel 422 (see, e.g., FIG. 11). The locking mechanism 416 utilizes friction between the retention elements 424 and the actuation member 406 to restrict relative movement therebetween, which retains the frame in a radially-expanded configuration.

In some examples, the actuation member 406 and/or the retention elements 424 can comprise one or more friction-increasing elements to secure the actuation member 406 relative to the locking mechanism 416. These friction-increasing elements can include forming the actuation member with a flat side oriented toward the retention elements, forming the retention elements to mate with the actuation member (e.g., C-shaped notches in the retention elements to engage a cylindrical outer surface of the actuation member, texturizing and/or coating the outer surface of the actuation member, teeth, holes, recesses, projections, and/or other means for increasing frictional engagement between the retention element and the actuation member. In one particular example, the actuation member can include ramped teeth configured to engage with the retention elements in a rachet-like mechanism, where the retention elements act as the pawl of the ratchet mechanism.

The locking mechanism 416 with the channel 422 and the retention elements 424 can be formed in various ways. In one example, the locking mechanism 416 can be formed by cutting a window or slot in the second vertically-oriented strut 414b (e.g., via a laser) such that the retention elements 424 are initially formed in a lateral configuration (i.e., transverse or orthogonal to the longitudinal axis of the second vertically-oriented strut 414b and the yet-to-be-formed channel 422), as depicted in FIG. 9A. The length of the retention elements is configured such that the retention elements intersect with the path of the yet-to-be-formed channel 422. As such, prior to formation of the channel 422, the retention elements 424 are deflected (either elastically or plastically) such that the retention elements are clear of the path where the channel 422 will be formed (see, e.g., FIG. 9B). With the retention elements 424 out of the way, the channel 422 can then be formed in the second vertically-oriented strut 416b (e.g., via electrical discharge machining (EDM) and/or electrochemical machining (ECM)). After the channel 422 is formed, the retention elements 424 can be returned to their lateral configuration (e.g., FIG. 9A). This can be accomplished, for example, by releasing the retention elements 424 from the angled orientation and allowing the bias of the retention elements 424 toward the lateral configuration to move the retention elements 424 relative to the second vertically-oriented strut 416b and the channel 422 from the deflected, angled orientation (FIG. 9B) to the resting, lateral configuration (FIG. 9A) where the retention elements 424 extend at least partially obstruct the channel 422.

In another example, the locking mechanism 416 can be formed by cutting a window or slot in the second vertically-oriented strut 414b (e.g., via a laser) such that the retention elements 424 are initially formed in the angled configuration (i.e., oblique) to the longitudinal axis of the second vertically-oriented strut 414b and the yet-to-be-formed channel 422, as depicted in FIG. 9B. The angle of the retention elements can be such that the retention elements do not obstruct the path of the yet-to-be-formed channel 422. In such instances, the channel 422 can be formed in the second vertically-oriented strut 416b (e.g., via EDM and/or ECM) either before or after the retention elements 424. After the channel 422 and the retention elements are formed, the retention elements 424 can be plastically deformed from the angled configuration (FIG. 9B) to the lateral configuration (FIG. 9A) in which the retention elements 424 intersect with the channel. This can be accomplished, for example, by moving the retention elements 424 relative to the second vertically-oriented strut 416b and the channel 422 from the angled orientation to the lateral orientation and shape-setting the retention elements 424 in the lateral configuration.

With the retention elements 424 and the channel 422 formed and the retention elements 424 in the lateral configuration (FIG. 9A), the actuation shaft 210 of the delivery apparatus 200 can be inserted through the channel 422 and coupled to the actuation member 406. In some examples, the actuation shaft 210 of the delivery apparatus comprises substantially the same diameter as the actuation member 406. In such examples, a proximal end of the actuation shaft 210 can be inserted into the channel 422 of the prosthetic valve 400 at the first end 426a of the second vertically-oriented strut 414b and advanced through the channel 422 to the second end 426b of the second vertically-oriented strut 414b. As the actuation shaft 210 moves through the channel 422, the actuation shaft 210 contacts the retention elements 424 of the prosthetic valve 400 and moves (elastically deforms) the retention elements from the lateral configuration to an angled configuration. In other examples, the actuation shaft of the delivery apparatus can comprise a relatively small diameter configured such that it does not contact the retention members of the prosthetic valve when passing through the channel 422. In such examples, the actuation shaft 210 can be inserted through the channel 422 and coupled to the actuation member 406 either by passing the proximal end of the actuation shaft 210 through the channel 422 from the first end 426a to the second end 426b of the second vertically-oriented strut 414b or by passing the distal end of the actuation shaft 210 through the channel 422 from the second end 426b to the first end 426a of the second vertically-oriented strut 414b. In these examples, the retention elements 424 will not move from the lateral configuration to the angled configuration until the actuation member 406 is drawn into the channel 422 and contacts the retention elements (e.g., during valve expansion).

The distal end portion of the actuation shaft 210 of the delivery apparatus 200 can be releasably coupled to the actuation member 406 of the prosthetic valve 400 in various ways. For example, as depicted in FIGS. 5, 8, and 11, the distal end portion of the actuation shaft 210 can comprise external threads 244 and the free end portion 406b of the actuation member 406 can comprise internal threads 428 (or vice versa), thereby enabling the actuation shaft 210 and the actuation member 406 to be threadably coupled together. In other examples, the actuation member 406 can comprise a lumen 430 (FIG. 10) extending axially therethrough, and the actuation shaft 210 can comprise a wire or suture that extends through the lumen 430 and is releasably secured to the outflow end portion of the frame 402 (e.g., looped around the first vertically-oriented strut 414a). Various other types of releasable connections between the actuation shaft 210 and the actuation member can be used. For example, FIGS. 45A-47 depict yet another example of how the actuation shafts of the delivery apparatus can be releasably coupled to the actuation member of the prosthetic valve.

With the actuation member 406 of the prosthetic valve releasably coupled to the actuation shafts of the delivery apparatus (see FIGS. 10-11), the delivery apparatus can be used to radially expand the prosthetic valve 400. The actuation shafts 210 of the delivery apparatus 200 can be used to apply a proximally-directed force on the inflow end 408 of the frame 402, and the support sleeves 208 of the delivery apparatus can be used to apply an opposing distally-directed force on the outflow end 410 of the frame 402. This axially-compressive force on the frame 402 causes the free end portion 406b of the actuation member 406 and the locking mechanism 416 to move towards each other. As noted above, in some examples, the actuation shaft of the delivery apparatus can be configured with a relatively small diameter such that the actuation shaft of the delivery apparatus does not engage the retention elements of the prosthetic valve as the actuation shaft moves through the channel of the prosthetic valve. This can allow the prosthetic valve to move between various radially-compressed states and radially-expanded states until the frame is radially expanded to the point at which the actuation member 406 enters the channel 422 and contacts the retention elements 424 of the locking mechanism 416. At this point, the frame 402 of the prosthetic valve 400 is locked and is prevented from radially compressing, though the frame 402 can be further radially expanded. In other examples, the actuation shaft 210 can be configured with a relatively large diameter such that the actuation shaft 210 engages the retention elements 424 as the actuation shaft moves through the channel 422. When the retention elements 424 contact the actuation member 406 (and/or the actuation shaft), the retention elements 424 deflect from the lateral configuration to the angled configuration (e.g., FIG. 11). In such instances, the frame of the prosthetic valve can be radially expanded but is prevented from radial compression.

Due to the angled orientation of the retention elements 424, the frictional engagement between the actuation member 406 and the retention elements 424 is less when moving the actuation member 406 proximally relative to the retention elements 424 than when attempting to move the actuation member 406 distally relative to the retention elements 424. As such, the actuation member 406 can continue to move proximally (and thus further radially expand) after initial engagement between the actuation member 406 and the retention elements 424, and the actuation member 406 is prevented from moving distally after the retention elements 424 of the locking mechanism 416 initially engage the actuation member 406 (and thus locking the frame 402 in an expanded configuration). Thus, the frame 402 of the prosthetic valve 400 can be locked in various radially-expanded configurations.

Once the frame 402 is radially-expanded to a desired diameter, the actuation shaft 210 of the delivery apparatus can be released from the actuation member 406 of the prosthetic valve 400 (e.g., by rotating the actuation shaft relative to the actuation member). The locking mechanism 416 retains the relative position of the actuation member 406 relative to the second vertically-oriented strut 414b, thereby securing the prosthetic valve 400 at the desired diameter.

The configurations of the frame, the actuation member, and/or the locking mechanism can be altered to change the predetermined diameter at which the actuation member engages the locking mechanism. For example, a relatively longer actuation member decreases the distance the free end portion of actuation member can move before engaging the locking mechanism (assuming the frame and the locking mechanism maintain a constant configuration). This results in the frame “locking” at a relatively smaller diameter than when the actuation member is relatively short. Additionally (or alternatively), the length of the vertically-oriented struts can be altered to change the predetermined diameter at which the actuation member engages the locking mechanism.

In some instances, the prosthetic valve can comprise a stopper configured to prevent further radial expansion of the frame. For example, the stopper can be a flange that extends radially outwardly from the actuation shaft and that is radially larger than the channel 422. Accordingly, the stopper cannot enter the channel 422 and abuts the first end 426a of the second vertically-oriented strut at a predetermined diameter (e.g., a maximum diameter). In this manner, the stopper prevents the actuation member from moving further in the proximal direction relative to the second vertically-oriented strut and therefore can help to prevent overexpansion of the frame.

FIGS. 12-14B depict a portion of a frame 500 of a prosthetic heart valve. The frame 500 can include a plurality (e.g., 3-15) of portions that are substantially similar to the portion depicted in FIG. 12. In certain instances, the frame comprises six portions (which may also be referred to as “regions” or “cells”).

In some examples, the frame 500 (as well as the other frames disclosed herein) can be a component of a prosthetic valve further comprising a valve structure and/or one or more sealing members. In other examples, the frame 500 (as well as the other frames disclosed herein) can be used as a docking station for a prosthetic valve that is deployed within the docking station. In yet other examples, the frames disclosed herein can be used as a stent or graft, which can, for example, be deployed in a blood vessel.

Referring to FIG. 12, the frame 500 comprises a plurality of pivoting struts 502, a plurality of non-pivoting struts 504, one or more actuation members 506, and one or more locking mechanisms 508. In the illustrated portion, the frame comprises one actuation member 506 and one locking mechanism 508. The frame can, in other examples, comprise 2-15 (or 3-12 or 3-6 or 6-9) pairs of actuation members and locking mechanisms (i.e., one actuation member and one locking mechanism forming one pair) distributed circumferentially about the frame. In particular examples, the frame can comprise 1, 2, 3, 4, 5, 6, 7, 8, 9, 10, 11, 12, 13, 14, or 15 pairs of actuation members and locking mechanisms. In some examples, the pairs of actuation members and locking mechanisms can be evenly spaced circumferentially relative to each other (e.g., 60 degrees between each adjacent pair of actuation members and locking mechanisms for a configuration comprising six pairs). In other examples, the pairs of actuation members and locking mechanisms can be non-evenly spaced circumferentially relative to each other.

The frame 500 is movable between one or more radially-compressed states and one or more radially-expanded states (e.g., via a delivery apparatus). When the frame expands or compresses radially, the pivoting struts 502 pivot relative to the non-pivoting struts 504. The pivoting struts 502 can also be referred to herein as “angled struts” due to the oblique angle the pivoting struts form relative to the non-pivoting struts. The angle between the pivoting struts 502 and the non-pivoting struts 504 increases as the frame 500 radially expands and axially foreshortens and decreases as the frame 500 radially compresses and axially elongates. The non-pivoting struts 504 can also be referred to as “vertically-oriented struts” or “axially-extending struts” due their depicted orientation and being generally parallel to the central longitudinal axis of the frame 500.

The actuation member 506 extends from a first non-pivoting strut 504a, and the locking mechanism 508 is formed in a second non-pivoting strut 504b. It should be noted that the non-pivoting struts are collectively or generically referred to as “the non-pivoting struts 504.”

The actuation member 506 of the frame 500 is configured similar to the actuation member 406 of the frame 402 in that the actuation member extends from the first non-pivoting strut 504a toward the second non-pivoting strut 504b and can be releasably coupled to a delivery apparatus (e.g., via threaded engagement, sutures, and/or other means for releasably coupling). When the frame is in a radially-compressed configuration and/or a partially radially-expanded configuration, the actuation member 506 is spaced apart from the locking mechanism 508. In embodiments where the actuation shaft of the delivery apparatus is sufficiently smaller than the actuation member of the prosthetic valve, the frame 500 can be move between various expanded/compressed states via a delivery apparatus and/or the bias (e.g., shape set) of the frame 500 to a particular state. The frame can be locked at one or more radially-expanded configurations by expanding the frame 500 to a point at which the actuation member 506 and the locking mechanism 508 engage one another. In the engaged configuration, the locking mechanism 508 prevents the actuation member from separating from the locking mechanism 508, thereby securing the frame in the expanded configuration.

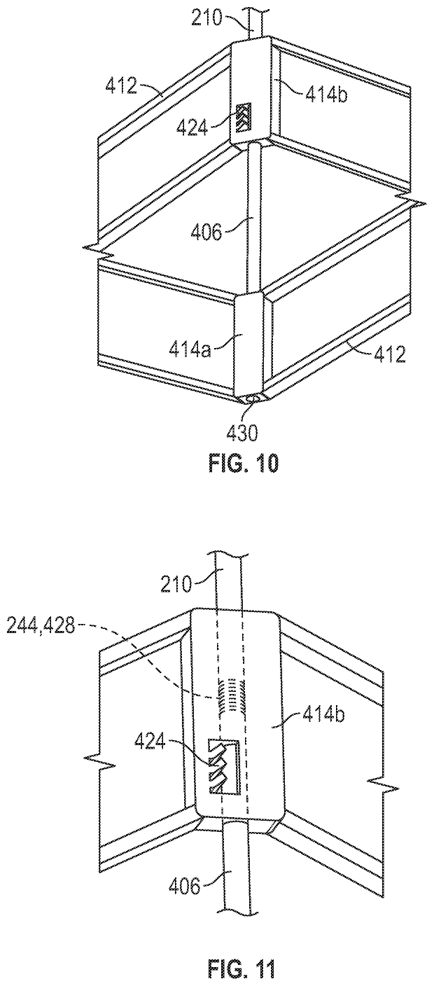

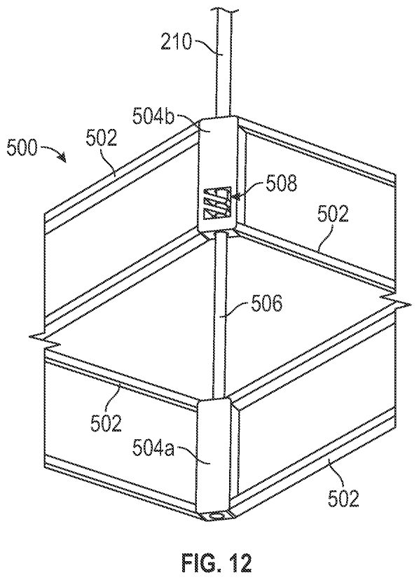

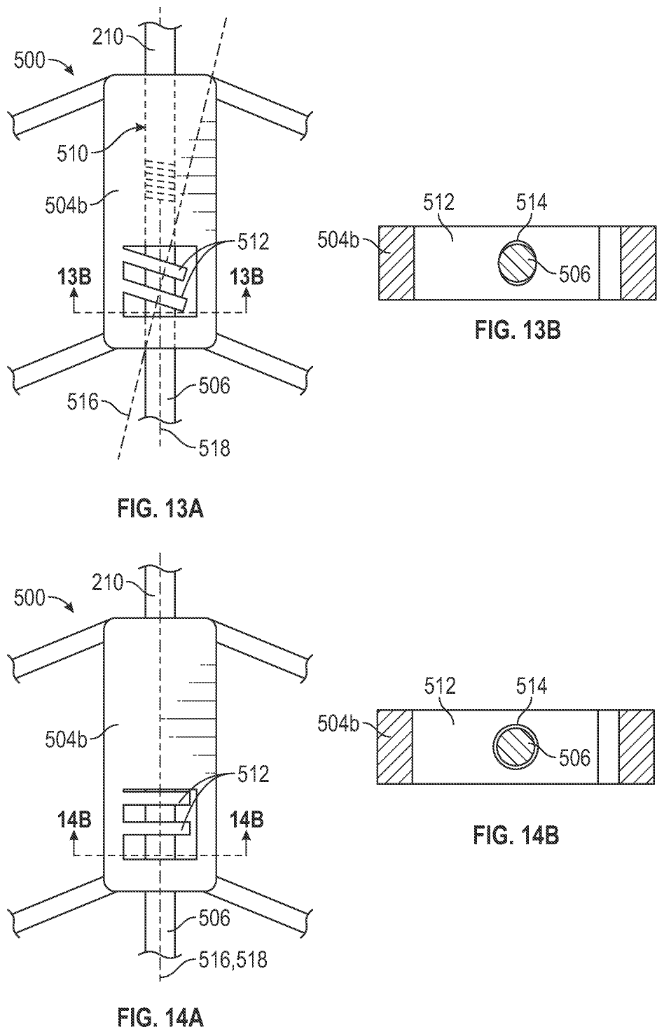

More specifically, as depicted in FIG. 13A, the locking mechanism 508 comprises a channel 510 and one or more retention elements 512 (e.g., two in the illustrated example). The channel 510 extends axially through the second non-pivoting strut 504b. The retention elements 512 each comprise an aperture 514 that can be aligned with the channel 510. The channel 510 and the apertures 514 are configured to receive the actuation shaft 210 of the delivery apparatus 200 and/or the actuation member 506 of the frame 500. The retention elements 512 are configured to engage the actuation member 506 to retain the position of the actuation member 506 relative to the second non-pivoting strut 504b, thereby locking the frame 500 in a radially-expanded configuration.

The retention elements 512 of the locking mechanism 508 are movable (e.g., pivotable) relative to the main portion of the second non-pivoting strut 504b from an angled (or locked) configuration (see FIG. 13A) to a lateral (or unlocked) configuration (see FIG. 14A). In the angled/locked configuration (FIG. 13A-13B), a central longitudinal axis 516 of the apertures 514 is tilted relative a central longitudinal axis 518 of the actuation member 506, which misaligns the apertures 514 relative to the actuation member 506. Thus, in the locked configuration (e.g., FIGS. 13A-13B), each retention element 512 contacts two portions of the outer surface of the actuation member 506, which are diametrically opposite from each other. The frictional engagement between the retention elements 512 and the actuation member 506 prevents the frame from returning to a smaller diameter. In the unlocked configuration (FIGS. 14A-14B), the central longitudinal axis 516 of the apertures 514 is coaxial (or at least substantially coaxial) with the central longitudinal axis 518 of the actuation member 506, which aligns the apertures 514 of the retention elements 512 with the actuation member 506 and reduces the frictional engagement between the retention elements 512 and the actuation member 506 such that the actuation member 506 can be moved in a direction corresponding to frame expansion (e.g., upward in the orientation depicted in FIG. 14A). Although FIG. 14B depicts a small gap between the actuation member 506 and the retention element 512, the retention elements 512 maintain at least some contact with the actuation member 506 when the retention elements 512 are in the unlocked state.

In some examples, the angle between the central longitudinal axis 516 of the apertures 514 and the central longitudinal axis 518 of the actuation member 506 when the retention members are in the locked configuration is oblique. In certain examples, the angle between the central longitudinal axis 516 of the apertures 514 and the central longitudinal axis 518 of the actuation member 506 when the retention members are in the locked configuration is within a range of 1-75 degrees or within a range of 5-50 degrees.

The retention elements 512 are biased to the locked/angled state by angling the retention elements “downward” (e.g., FIG. 13A). In other words, free end portions of the retention elements 512 (i.e., the portions with the apertures 514 formed therein) are disposed closer to the first non-pivoting strut 504a (FIG. 12) than fixed end portions of the retention elements 512 (i.e., the portions coupled to the second non-pivoting strut 504b) when the retention elements 512 are in a resting or undeflected state. In this manner, the retention elements frictionally engage (or “bite”) the actuation member 506 and restrict radial compression of the frame once the retention elements 512 initially engage the actuation member 506. When the retention elements 512 are engaged with the actuation member 506 and the actuation member 506 moves proximally (e.g., upward) relative to the second non-pivoting strut 504b, the retention elements 512 initially move proximally together with the actuation member 506, thereby moving the retention elements 512 from the locked configuration (FIG. 13A) to the unlocked configuration (FIG. 14A). The retention elements move proximally together with the actuation member until the proximal force on the actuation member is at least slightly greater than the frictional force of the retention elements on the actuation member. The retention elements can be configured such that this “unlocking” occurs when the retention members are horizontal or substantially horizontal (e.g., within a few degrees of horizontal).

When the retention elements 512 are in the unlocked configuration, the actuation member 506 is aligned with the apertures 514 of the retention elements 512 can slide proximally through the apertures 514 of the retention elements 512, which results in radial expansion of the frame 500. As mentioned above, the retention elements 512 maintain contact with the actuation member 506 in the unlocked state, but the contact does not provide sufficient friction to prevent the actuation shaft from moving proximally relative to the retention elements 512. In some instances, the proximal-most retention member can be configured to contact the proximal wall of the opening in which the retention elements are formed. The proximal wall can thus prevent the retention elements from angling upward (i.e., opposite the downward angle depicted in FIG. 13A), which could result in the actuation member being prevented from moving proximally relative to the retention elements and thus prevent expansion of the frame.

When the proximal force on the actuation member 506 reduced such that it is less than or equal to the frictional force of the retention elements 512 on the actuation member, the actuation member stops moving axially (proximally or distally) relative to the retention elements, thereby locking the frame in a particular expanded state (i.e., diameter). When the proximal force on the actuation member is less than the distal force exerted by the retention elements to their biased/angled state, the retention elements and the actuation member move together slightly distally until the retention elements reach their resting position. It should be noted that the frictional engagement between the retention elements and the actuation member increases as the retention members move from the horizontal configuration (FIG. 14A) to their resting configuration (FIG. 13A).

The depicted example comprises two retention elements 512. In other examples, a locking mechanism can comprise one retention element. In yet other examples, a locking mechanism can comprise more than two (e.g., 3-20) retention elements.

The frame 500 (and/or the other frames disclosed herein) can, in some examples, be formed of a shape memory material (e.g., nitinol) such that the frame can be shape-set at a particular configuration and then elastically deformed to one or more other configurations. For example, the frame can be shape-set in the partially radially-expanded configuration and can be elastically deformed to the radially-compressed configuration and/or to the radially-expanded configuration (e.g., FIG. 2A). The frame can be elastically deformed to the various configurations, for example, by using the actuator members, a delivery apparatus, and/or a crimping device), as further described below.

In some examples, the frame can be formed from a single piece of material (e.g., a metal tube). This can be accomplished, for example, via laser cutting, electroforming, and/or physical vapor deposition. In other examples, the frame can be constructed by forming individual components coupling the individual components together (e.g., via welding).

The locking mechanism 508 of the frame 500 can be formed of a shape memory material (e.g., nitinol) and can be formed in various ways. In one example, a window or slot can be formed in the second non-pivoting strut 504b (e.g., via laser cutting) to form the retention elements 512 in the lateral configuration (e.g., FIG. 14A). The channel 510 can then be formed in the second non-pivoting strut 504b and the retention elements 512 (e.g., via EDM, ECM, and/or drilling). In another example, the channel 510 can be formed first, and then the retention elements 512 can be formed. In either instance, after the channel 510 and the retention elements 512 are formed, the retention elements can be moved from the lateral configuration (FIG. 14A) to the angled configuration (FIG. 13A) and shape-set in the angled configuration (e.g., via heat setting).

The actuation member 506 of the frame 500 can be releasably coupled to a delivery apparatus in various ways. For example, as depicted in FIG. 13A, an end portion of the actuation member can comprise a threaded connection with the actuation shaft 210. Additionally (or alternatively), sutures, interlocking shafts, and/or other means for releasably coupling can be used.

In some examples, the delivery apparatus 200 can be used to delivery, position, axially compress, and/or radially expand the frame 500. In such instances, the actuation shaft 210 can be releasably coupled to the actuation member 506, and the support sleeves 208 can be used to apply an opposing force to the frame 500 when the actuation shaft 210 and actuation member are tensioned during frame expansion.

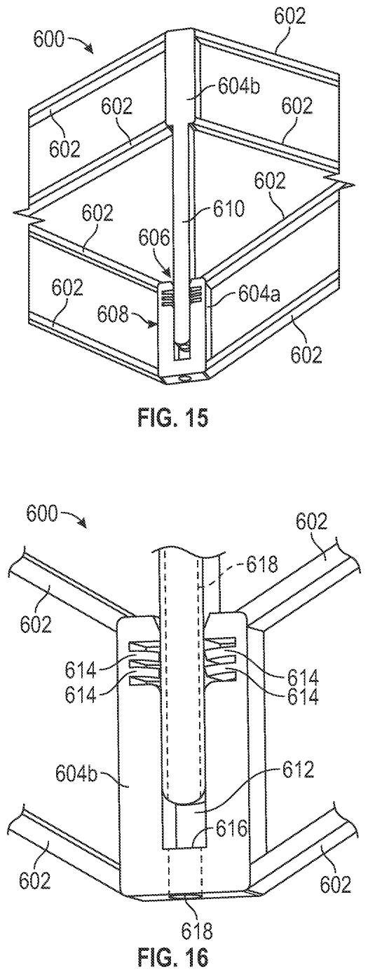

FIGS. 15-17D depict a portion of a frame 600 of a prosthetic heart valve. The frame 600 can include a plurality (e.g., 3-15) of regions that are substantially similar to the region depicted in FIG. 15. In certain instances, the frame comprises six regions (which may also be referred to as “portion” or “cells”).

Referring to FIG. 15, the frame 600 comprises a plurality of pivoting struts 602, a plurality of non-pivoting struts 604a and 604b (which are collectively or generically referred to as “the non-pivoting struts 604”), and a locking mechanism 606. In the illustrated portion, the frame comprises one locking mechanism 606. The frame can, in other examples, comprise 2-15 locking mechanisms distributed circumferentially about the frame. In particular examples, the frame can comprise 3, 6, 9, 12, or 15 locking mechanisms. In some examples, the locking mechanisms can be evenly spaced circumferentially relative to each other (e.g., 60 degrees between adjacent locking mechanisms for a configuration comprising six locking mechanisms). In other examples, the locking mechanisms can be non-evenly spaced circumferentially relative to each other.

The frame 600 is movable between one or more radially-compressed states and one or more radially-expanded states (e.g., via a delivery apparatus), as depicted, for example, in FIGS. 17A-17D. When the frame 600 expands or compresses radially, the pivoting struts 602 pivot relative to the non-pivoting struts 604. The pivoting struts 602 can also be referred to herein as “angled struts” due to the oblique angle the pivoting struts form relative to the non-pivoting struts 604. The angle between the pivoting struts 602 and the non-pivoting struts 604 increases as the frame 600 radially expands and axially foreshortens and decreases as the frame 600 radially compresses and axially elongates. The non-pivoting struts 604 can also be referred to herein as “vertically-oriented struts” or “axially-extending struts” due their depicted orientation and being generally parallel to the central longitudinal axis of the frame 600.

The locking mechanism 606 is configured to retain the frame 600 in a desired radially expanded configuration. Referring to FIGS. 15-16, the locking mechanism 606 comprises a female portion 608 and a male portion 610. The female portion 608 of the locking mechanism 606 is formed in a first non-pivoting strut 604a and comprises a slot 612 and one or more retention elements 614 (e.g., four in the depicted example). The male portion 610 of the locking mechanism 606 is coupled to and extends axially from a second non-pivoting strut 604b toward the first non-pivoting strut 604a. In the depicted example, the female portion 608 of the locking mechanism 606 and the first non-pivoting strut 604a are disposed adjacent an inflow end of the frame 600, and the male portion 610 of the locking mechanism 606 and the second non-pivoting strut 604b are disposed adjacent an outflow end of the frame 600. In other examples, the female portion of the locking mechanism and/or the first non-pivoting strut can be disposed adjacent an outflow end of the frame, and the male portion of the locking mechanism and/or the second non-pivoting strut can be disposed adjacent an inflow end of the frame.

When the frame is in a radially-compressed configuration (e.g., FIG. 17A) or a partially radially-expanded configuration (e.g., FIG. 17B), the male portion 610 of the locking mechanism 606 is axially spaced from the female portion 608 of the locking mechanism 606. As the frame 600 radially expands, the female portion 608 and the male portion 610 move axially toward each other. At a predetermined point the female portion 608 and the male portion 610 engage with each other as the male portion 610 enters the slot 612 of the female portion 608. The retention elements 614, which protrude into the slot 612, engage the male portion 610. The frictional engagement between the retention elements 614 and the male portion 610 secures the frame 600 in a radially-expanded configuration. The frame 600 can be further radially expanded and is prevented from radially contracting.

The frame 600 can expand radially until the end of the male portion 610 contacts an end surface 616 (FIG. 16), as depicted in FIG. 17D. The length of the male portion 610 and/or the depth of the slot 612 of the female portion 608 (which is defined by the end surface 616) can, for example, be configured to prevent overexpansion of the frame 600. For example, if the frame 600 is configured to be radially expanded to nominal diameter of 26 mm and to a maximum diameter of 29 mm, the female portion 608 and the male portion 610 of the locking mechanism can be configured to initial engage and thus prevent radial compression when the frame is expanded to a diameter which is slightly less than the nominal diameter (e.g., 24-25 mm). The frame 600 can then be further expanded to the nominal diameter. If desired (e.g., to reduce paravalvular leakage (PVL), the frame 600 can be expanded from the nominal diameter to the maximum diameter. At the maximum diameter, the end of the male portion 610 contacts the end surface 616, which prevents further radial expansion of the frame 600. In this manner, the locking mechanism 606 serves as a “stopper.” This can advantageously prevent the frame 600 from being overexpanded, which thus reduces the likelihood of damage to the frame 600 and/or to the native tissue (e.g., a native annulus) in which the frame 600 is expanded.

Referring again to FIG. 16, the female portion 608 of the locking mechanism 606 comprises two retention elements 614 on each side of the slot 612. In other examples, the female portion 608 can comprise less (e.g., one) or more (e.g., 3-15) retention elements on each side of the slot. In the depicted example, the female portion 608 comprises the same number of retention elements (e.g., two) on each side of the slot 612. In other examples, the female portion can comprise a different number of retention elements on a first side of the slot than on a second side of the slot. The retention elements on either side of the slot can be axially aligned (as depicted) or axially offset.

Referring still to FIG. 16, the male portion 610 of the locking mechanism 606 comprises an elongate shaft. The shaft can comprise various shapes. For example, as depicted, the shaft comprises a rectangular cross-sectional profile taken in a plane perpendicular to the longitudinal axis of the shaft. The flat sides of the shaft directed toward the retention elements 614 can, for example, provide increased surface area contact between the shaft and the retention elements (compared to a cylindrical shaft), which therefore more securely locks the frame in a desired configuration. In other examples, the shaft can comprise a circular, ovular, triangular, or other cross-section profiles, including non-standard geometric shapes.

The frame 600 further comprises a lumen 618 which extends from an inflow end to the outflow end of the frame, passing through the non-pivoting struts 604 and the male portion 610 of the locking mechanism 606. The lumen 618 is aligned (e.g., coaxial) with the slot 612 of the female portion 608 of the locking mechanism 606. The lumen 618 can be configured to receive an actuation shaft 210 of the delivery apparatus 200, as depicted in FIGS. 17A-17D. In some examples, the portion of the lumen 618 formed in the first non-pivoting strut 604a can comprise internal threads configured to mate with the external threads 244 (FIG. 5) of the actuation shaft 210 of the delivery apparatus 200. In other examples, the actuation shaft of the delivery apparatus can comprise a suture or a wire that extends through the lumen 618 and is releasably secured to the inflow end of the frame 402, e.g., looped around the first non-pivoting strut 604. Regardless of the manner of coupling, the actuation shaft of the delivery apparatus, which is releasably coupled to the inflow end of the frame 600, can be used to apply a proximally-directed force on the frame, and the support sleeve, which is releasably coupled to the outflow end of the frame 600, can be used to apply an opposing, distally-directed force on the frame. These opposing axial forces can be used to radially expand the frame 600. Once the frame 600 is expanded to a desired diameter and secured in the desired diameter with the locking mechanism 606, the delivery apparatus can be released from the frame.

The frame 600 can be formed in various ways. For example, the frame can be formed by laser cutting the pivoting struts 602, the non-pivoting struts 604, and the locking mechanism 606 from a tube (e.g., a metal tube, such as nitinol). The frame 600 can be formed in a configuration in which the male portion of the locking mechanism is axially spaced from the female portion of the locking mechanism (e.g., FIGS. 17A-17B). As such, the frame 600 is relatively easy to manufacture since most of the frame formation can be done in a single process (e.g., via laser cutting). Before or after the struts and locking mechanism of the frame are formed, the lumen 618 can be formed (e.g., via EDM and/or ECM).

FIGS. 18-20B depict a portion of a frame 700 of a prosthetic heart valve. The frame 700 can include a plurality (e.g., 3-15) of regions that are substantially similar to the region depicted in FIG. 18. In certain instances, the frame comprises six regions (which may also be referred to as “portion” or “cells”). The frame 700 comprises a plurality of pivoting struts 702, a plurality of non-pivoting struts 704a and 704b (which are collectively or generically referred to as “the non-pivoting struts 704”), an actuation member 706, and a locking mechanism 708.

The frame 700 is movable between one or more radially-compressed states and one or more radially-expanded states (e.g., via a delivery apparatus) and functions generally similar to the other frames described above. One difference between the frame 700 and the previously-described frames is the locking mechanism 708.

The locking mechanism 708 is an elongate tab (or “arm”) that extends from the second non-pivoting strut 704b toward the first non-pivoting strut 704a. The locking mechanism comprises a wave shape (or “C” shape) that intersects with the actuation member 706 at two locations. The locking mechanism 708 comprises two apertures 710 that are spaced apart from each other and configured to allow the actuation member 706 to pass therethrough. In other examples, the locking mechanism 708 can comprise fewer (e.g., 1) or more (e.g., 2-15) apertures and can comprise various other shapes (e.g., a “V” shape).

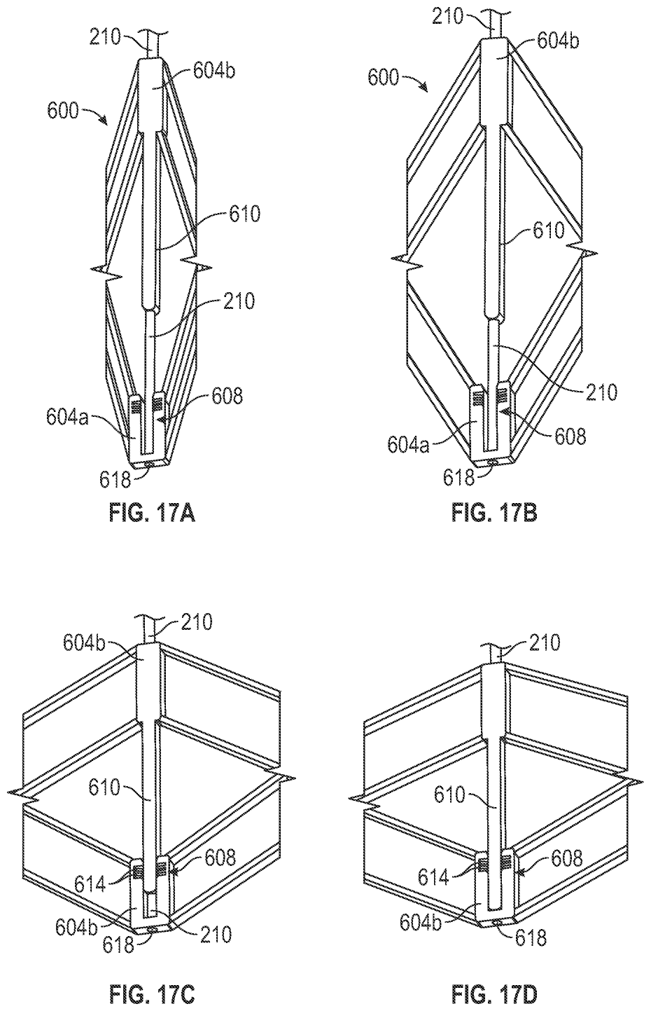

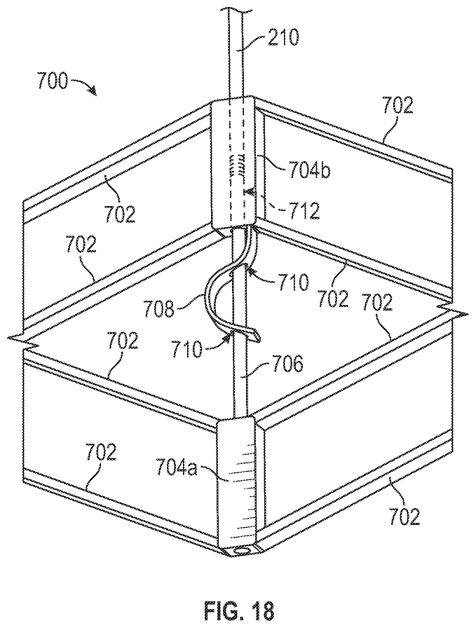

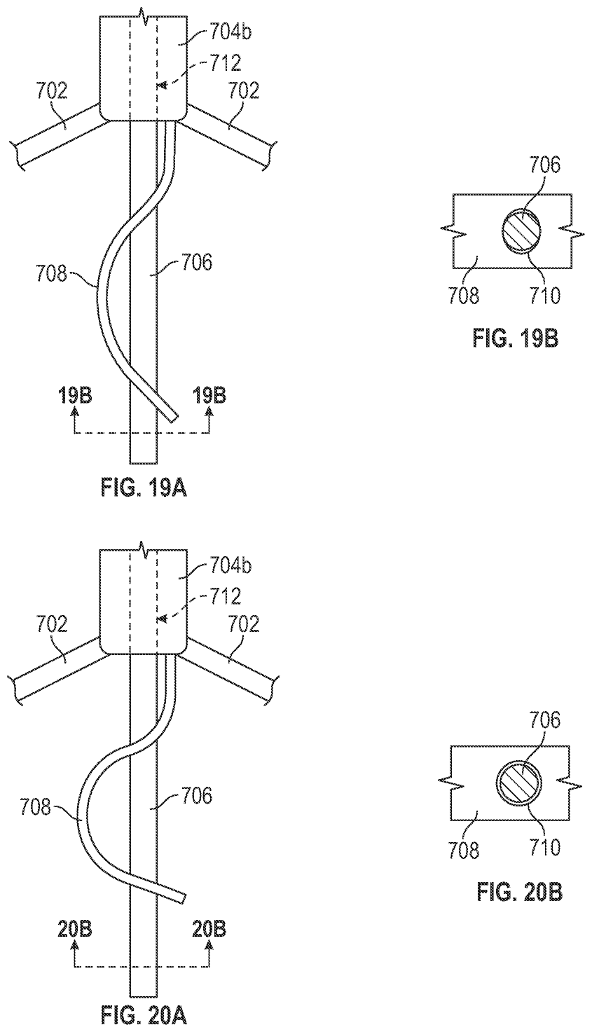

The locking mechanism 708 is biased to a locked configuration in which the apertures 710 are tilted relative to the actuation member 706, as depicted in FIGS. 19A-19B. In other words, the apertures 710 are not coaxial with the actuation member 706. In the locked configuration, each locking mechanism engages two portions of the actuation member 706 (i.e., on two “sides” of the actuation member), as depicted in FIG. 19B. The frictional engagement between the locking mechanism and the actuation member is sufficient to prevent the actuation member 706 from moving in a direction corresponding to radial compression of the frame (e.g., downward in the depicted orientation). The greater the force exerted on the frame 700 attempting to radially compress the frame 700, the more misaligned the apertures of the locking mechanisms and the actuation member become, which increases the frictional engagement between the locking mechanism 708 and the actuation member 706.

The locking mechanism 708 is movable from the locked configuration to an unlocked configuration (e.g., FIGS. 20A-20B) by moving the actuation member 706 in a direction corresponding to radial expansion of the frame 700 (e.g., upward in the depicted orientation). As actuation member 706 moves upward relative to the second non-pivoting strut 704b, the locking mechanism 708 initially moves upward together with the actuation member 706. This causes the locking mechanism to axially foreshorten (compare FIGS. 19A and 20A) and the apertures 710 to better align with the actuation member 706 (compare FIGS. 19B and 20B). Also, the curvature of the locking mechanism is tighter (e.g., has a smaller radius) when the locking mechanism is in the unlocked configuration compared to the locked configuration. Due to the better alignment (and therefore reduced friction), the actuation member 706 can move axially through the apertures 710 of the locking mechanism 708 in the proximal direction, which radially expands the frame 700. When proximal force on the actuation member 706 is reduced such that the proximal force is less than or equal to the frictional force of the locking mechanism 708 on the actuation member 706, the actuation member 706 stops moving relative to the locking mechanism 708. When the proximal force on the actuation member 706 is less than the distal force of the locking mechanism 708 to its biased configuration, the locking mechanism 708 and the actuation member move slightly distally until the locking mechanism reaches its resting/locked configuration (FIG. 19A), thereby securing the frame at a particular expanded configuration. The frame can be further expanded by repeating the process.

The actuation member 706 can be releasably coupled to a delivery apparatus in various ways. For example, as depicted in FIG. 18, the second non-pivoting strut 704b can comprise a lumen 712 through which the actuation member 706 and/or the actuation shaft 210 can extend, and the actuation member 706 can be releasably coupled to the actuation shaft 210 of the delivery apparatus via a threaded connection. In this manner, the actuation shaft 210 can be used to apply a proximally-directed force on the actuation member 706, and the support sleeve 208 can be used to apply an opposite, distally-directed force on the second non-pivoting strut 704b, which results in radial expansion of the frame 700.

One advantage of the frame 700 is that the frame can be locked in a relatively wide range of diameters. This is because the locking mechanism 708 engages the actuation member 706 across a large portion of the overall length of the actuation member (compared to some examples where the actuation member is sometimes disengaged with the locking mechanism).

The frame 700 can be formed, for example, by laser cutting the struts 702, 704, the actuation member 706, and the locking mechanism 708 from a tube. The tube can comprise a shape memory material (e.g., nitinol). The frame can be formed in a configuration in which the actuation member 706 is axially spaced apart (e.g., disengaged) from the locking mechanism 708. The locking mechanism 708 can be formed in the unlocked configuration. The lumen 712 and the apertures 710 can be formed, for example, using EDM and/or ECM, either before or after the struts, the actuation member, and/or the locking mechanism are formed. After the locking mechanism 708 and the apertures 710 are formed, the locking mechanism 708 can be moved from the unlocked configuration to the locked configuration and shape set in the locked configuration.

In other examples, the actuation member can be formed as a separate component from the frame 402, and the actuation member can be fixedly coupled to the frame (e.g., via a fastener, welding, adhesive, etc.).

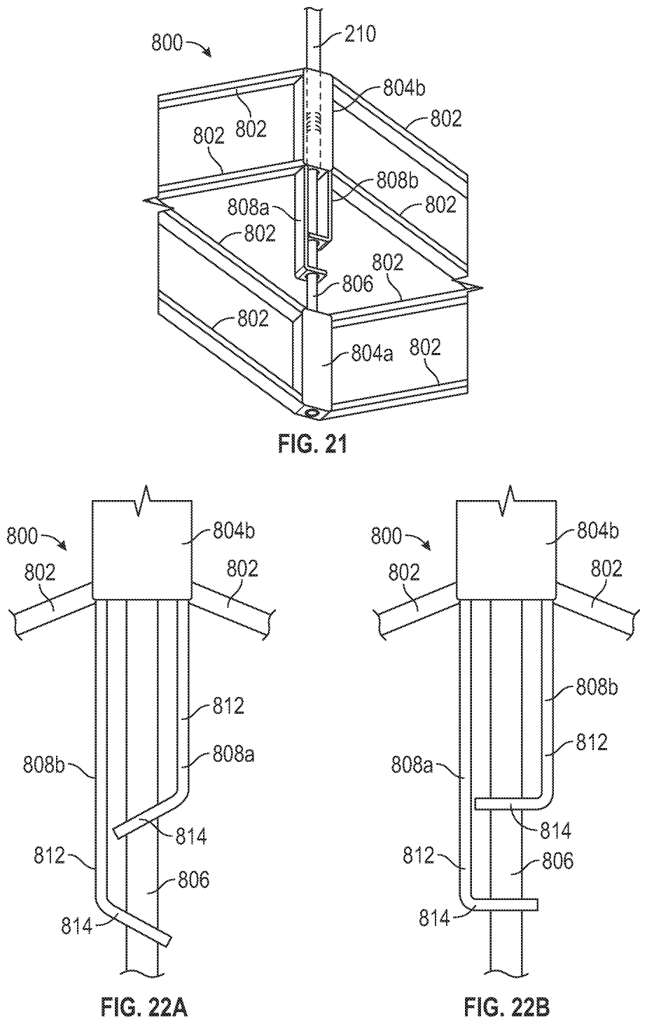

FIGS. 21-22B depict a portion of a frame 800 of a prosthetic heart valve. The frame 800 can include a plurality (e.g., 3-15) of regions that are substantially similar to the region depicted in FIG. 21. In certain instances, the frame comprises six regions (which may also be referred to as “portion” or “cells”). The frame 800 comprises a plurality of pivoting struts 802, a plurality of non-pivoting struts 804a and 804b (which are collectively or generically referred to as “the non-pivoting struts 804”), an actuation member 806, and a locking mechanism (which includes a first locking member 808a and a second locking member 808b, collectively or generically referred to as “the locking members 808”). In other examples, the frame can comprise a single locking member or more than two locking members.

The frame 800 is movable between one or more radially-compressed states and one or more radially-expanded states (e.g., via a delivery apparatus) and functions generally similar to the other frames described above. One difference between the frame 800 and the previously-described frames is the locking mechanism.

The locking mechanism of the frame 800 includes the locking members 808 extending from the second non-pivoting strut 804b toward the first non-pivoting strut 804a, the first locking member 808a comprising an “L” shape, and the second locking member 808b comprising a “J” shape. In the depicted example, the first locking member 808a is longer than the second locking member 808b. In other examples, the first locking member is shorter than the second locking member.

Each locking member 808 comprises an extension portion 812 and an engagement portion 814. The extension portion 812 is coupled to the second non-pivoting strut 804b and is (or at least substantially) parallel to the actuation member 806. The engagement portion 814 extends from the extension portion 812 at an oblique angle. The engagement portion 814 of each locking member 808 comprises an aperture configured to maintain contact with the actuation member and to allow the actuation member 806 to pass therethrough when in the engagement portion is in an unlocked configuration (FIG. 22B). The engagement portions 814 of the locking members 808 are also configured to contact the actuation member 806 with sufficient frictional force to secure the frame 800 in a desired configuration.

The engagement portions 814 of the locking members 808 are biased to a locked configuration, as depicted in FIG. 22A. In the locked configuration, the engagement portions 814 (and thus the apertures) of the locking member 808 are tilted relative to the actuation member 806. Therefore, in the locked configuration, the engagement portions 814 of the locking members 808 each engage two “sides” of the actuation member in a manner similar to that depicted in FIG. 19B with the frame 700. The frictional engagement between the locking members 808 and the actuation member 806 is sufficient to prevent the actuation member 806 from moving in a direction corresponding to radial compression of the frame (e.g., downward in the depicted orientation). The greater the force exerted on the frame 800 attempting to radially compress the frame 800, the more misaligned the apertures of the locking members 808 and the actuation member 806 become, which increases the frictional engagement between the locking members 808 and the actuation member 806.

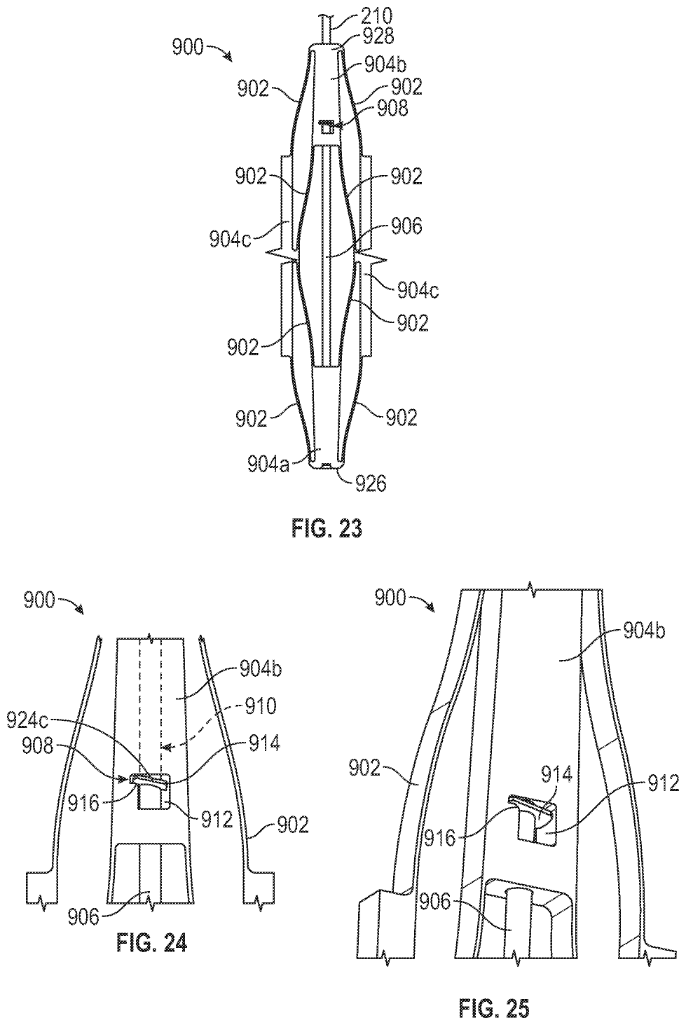

The locking members 808 are movable from the locked configuration (FIG. 22A) to an unlocked configuration (FIG. 22B) by moving the actuation member 806 in a direction corresponding to radial expansion of the frame 800 (e.g., upward in the depicted orientation). As actuation member 806 moves upward relative to the second non-pivoting strut 804b, the engagement portions 814 of the locking members 808 initially move together with the actuation member 806 due to the frictional engagement between the two components. This results in the engagement portions 814 of the locking members 808 pivoting relative to the extension portions 812 of the locking members 808 such that the apertures of the locking members 808 better align with the actuation member 806. Improving the alignment reduces the friction between the locking members 808 and the actuation member 806 such that the actuation member 806 can move upwardly through the apertures of the locking members 808 to radially expand the frame 800. When the proximal force on the actuation member is less than the frictional force on the actuation member from the locking members, the actuation member 806 stops moving relative to the locking members 808. The engagement portions 814 of the locking members 808 return to the locked configuration (FIG. 23) due to their bias to the locked configuration when the proximal force on the actuation member is less than the distal force from the bias of the locking members to the locked configuration.

The actuation member 806 can be releasably coupled to a delivery apparatus in various ways. For example, as depicted in FIG. 22, the second non-pivoting strut 804b can comprise a lumen through which the actuation member 806 and/or the actuation shaft 210 can extend, and the actuation member 806 can be releasably coupled to the actuation shaft 210 of the delivery apparatus via a threaded connection. In this manner, the actuation shaft 210 can be used to apply a proximally-directed force on the actuation member 806, and the support sleeve 208 can be used to apply an opposite, distally-directed force on the second non-pivoting strut 804b, which results in radial expansion of the frame 800.

One advantage of the frame 800 is that the frame can be locked in a relatively wide range of diameters. This is because the locking members 808 engage the actuation member 806 across a large portion of the overall length of the actuation member (compared to some examples where the actuation member is sometimes disengaged with the locking mechanism).

The frame 800 can be formed in a manner similar to that of the frame 700. For example, the struts 802, 804, the actuation member 806, and the locking mechanisms 808 can be laser-cut from a metal (e.g., nitinol tube), and the lumen and apertures (e.g., for receiving the actuation shaft and/or actuation member) can be formed via EDM and/or ECM.

FIGS. 23-28C depict a portion of a frame 900 of a prosthetic heart valve and/or its components. The frame 900 can include a plurality (e.g., 3-15) of regions that are substantially similar to the region depicted in FIG. 23. In certain instances, the frame comprises six regions (which may also be referred to as “portion” or “cells”). The frame 900 comprises a plurality of pivoting struts 902, a plurality of non-pivoting struts 904a, 904b, and 904c (which are collectively or generically referred to as “the non-pivoting struts 904”), an actuation member 906, and a locking mechanism 908.

The actuation member 906 is fixedly coupled to the first non-pivoting strut 904a and extend axially toward the second non-pivoting strut 904b. The actuation member 906 can be fixedly coupled to the first non-pivoting strut 904a in various ways, including integrally forming the actuation member 906 and the first non-pivoting strut 904a from as a single, unitary component (e.g., from a metal tube) or by forming the actuation member 906 as a separate component from the first non-pivoting strut 904a (and the rest of the frame 900) and fixedly coupling the actuation member 906 to the first non-pivoting strut 904a (e.g., via welding, fasteners (e.g., screws, sutures, etc.), adhesive, and/or other means for coupling).

Referring to FIGS. 23-25, the second non-pivoting strut 904b comprises a lumen 910 extending axially therethrough. The lumen 910 is configured to receive the actuation member 906 and/or the actuation shaft 210 of the delivery apparatus 200.

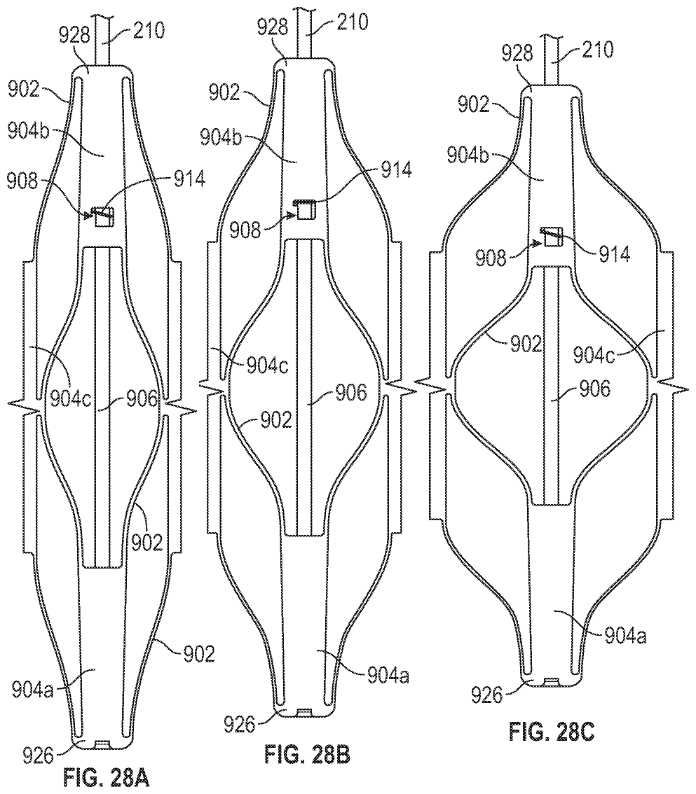

As depicted in FIGS. 28A-28C, the frame 900 is movable between one or more radially-compressed states and one or more radially-expanded states (e.g., via a delivery apparatus) and functions generally similar to the other frames described above. One difference between the frame 900 and the previously-described frames is the locking mechanism 908.

Referring to FIGS. 24-25, the locking mechanism 908 comprises a window 912 formed in the second non-pivoting strut 904b and a locker disc 914 disposed within the window 912. The window 912 comprises a support shoulder 916 extending laterally from one side. The support shoulder 916 is configured to engage the locker disc 914 and bias the locker disc 914 to a locked, angled configuration. The locker disc 914 can be wedged in the window and extend from the support shoulder 916 in a cantilevered configuration. This allows the locker disc 914 to pivot about the support shoulder 916 between the locked, angled configuration (e.g., FIG. 28A) to an unlocked, lateral configuration (e.g., FIG. 28B), as further described below.

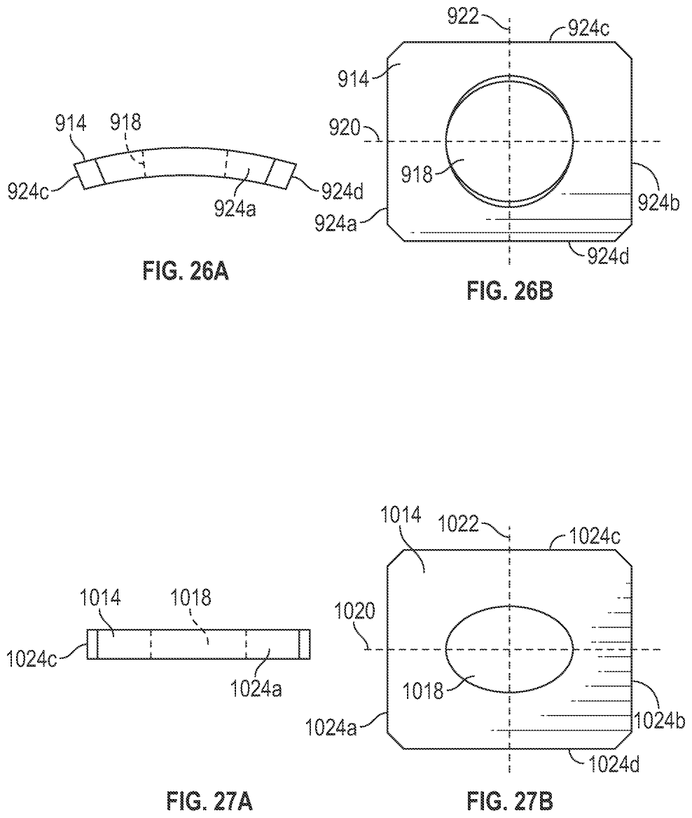

Turning to FIGS. 26A-26B, the locker disc 914 comprises an opening 918 configured for receiving the actuation member 906 and/or the actuation shaft 210. The locker disc 914 is formed from a flat plate and the opening 918 having a circular shape is formed therein. After formation of the opening 918, the locker disc 914 can be plastically deformed from the flat configuration to a curved configuration. In the curved configuration, the opening 918 of the locker disc 914 effectively has an ovular shape having a major axis 920 and minor axis 922, as depicted in FIG. 26B. The major axis 920 extends from a first side 924a to a second side 924b of the locker disc 914, the first and second sides being opposite each other. The minor axis 922 extends from a third side 924c to a fourth side 924d of the locker disc, the third and fourth sides being opposite each other and substantially perpendicular to the first and second sides. Alternatively, a flat locker disc 1014 having an ovular opening 1018 can be used, as depicted in FIGS. 27A-27B. For the sake of brevity, the locker disc 1014 is labeled with reference numbers that correspond to components of the locker disc 914, but the reference numbers are increased by 100. The opening (e.g., the opening 918, 1018) in the locker disc (e.g., the locker disc 914, 1014) is configured such that the sides of the locker disc disposed along the minor axis (e.g., the minor axis 922) remain engaged with the actuation member in both a locked configuration and an unlocked configuration, as further explained below. Accordingly, the locker disc moves together with the actuation member unless movement of the locker disc is restricted by another object (e.g., a non-pivoting strut of the frame).

Referring to FIGS. 24-25, the locker disc 914 is disposed in the window 912 such the first side 924a and the second side 924b of the locker disc 914 are oriented in the lateral/circumferential direction (left/right in the depicted orientation) and such the third side 924c and the fourth side 924d of the locker disc 914 are oriented in the radial direction (in/out in the depicted orientation).

FIGS. 28A-28C depict the frame 900 radially expanding and the locking mechanism moving from a locked configuration (FIG. 28A) to an unlocked configuration (FIG. 28B) and back to the locked configuration (FIG. 28C). In the locked configuration, the opening 918 of the locker disc 914 is tilted or angled along its major axis 920 (FIG. 26) relative to the longitudinal axis of the actuation member 906. In some examples, this angle can be within a range of 5-85 degrees or 10-65 degrees. The locker disc 914 is also wedged in the window 912 such that the locker disc 914 is restricted from moving toward a first end 926 of the frame 900. Due to the tilting, the locker disc 914 engages the actuation member 906 along the major axis 920 of the opening 918. Due to the ovular shape of the opening 918, the locker disc 914 also engages the actuation member 906 along the minor axis 922 of the opening 918. The bias of the frame 900 toward a smaller radial configuration (and/or an inward force from a patient's native anatomy) maintains tension on the actuation member 906, which in turn holds the locker disc 914 against the support shoulder 916 in the locked, angled configuration. In this manner, the locking mechanism 908 prevents the frame 900 from radially compressing.

When the actuation member 906 moves axially toward a second end 928 of the frame 900, the locker disc 914 initially moves axially together with the actuation member 906. The locker disc 914 pivots (e.g., upward in the depicted orientation) about the support shoulder 916 and moves from the locked, angled configuration to the unlocked, lateral configuration, as depicted in FIG. 28B. The locker disc 914 continues to move axially with the actuation member 906 until the locker disc 914 contacts the end wall of the window 912 disposed closest to the second end 928 of the frame 900. At this point, the locker disc 914 is restricted from further axial and/or pivoting movement toward the second end 928 of the frame 900. The actuation member 906 can continue to move axially relative to the locker disc 914 and the second non-pivoting strut 904b toward the second end 928 of the frame 900 by sliding through the opening 918 of the locker disc 914 and the lumen 910 of the second non-pivoting strut 904b, which results in radial expansion of the frame 900. In the unlocked state, the locker disc 914 maintains some contact with the actuation member 906 along the minor axis 922 of the opening 918, but the contact is insufficient to prevent the frame 900 from being radially expanded.

The frame 900 can be locked at a desired diameter by reducing the tensile force on the actuation member 906 (e.g., via the actuation shaft 210) such that it is less than the opposing force of the frame 900 attempting to return to the radially-compressed configuration (i.e., due to the bias of the frame to the radially-compressed state and/or due to forces on the frame from the native anatomy). This results in the actuation member 906 and the locker disc 914 moving slightly axially toward the first end 926 of the frame 900. The locker disc 914 contacts and pivots (e.g., downward in the depicted orientation) about the support shoulder 916 and moves from the lateral, unlocked configuration to the angled, locked configuration, as depicted in FIG. 28C.

The actuation member 906 can be releasably coupled to a delivery apparatus in various ways. For example, the actuation member 906 can be releasably coupled to the actuation shaft 210 of the delivery apparatus via a threaded connection. In this manner, the actuation shaft 210 can be used to apply a proximally-directed force on the actuation member 906, and the support sleeve 208 can be used to apply an opposite, distally-directed force on the second non-pivoting strut 904b, thereby producing radial expansion of the frame 900. The frame 900 (as well as the other frame disclosed herein) can also be configured to for use with other types of releasable connections between the actuation member of the frame and an actuation shaft (or shafts) of a delivery apparatus (e.g., the delivery apparatus 2100)

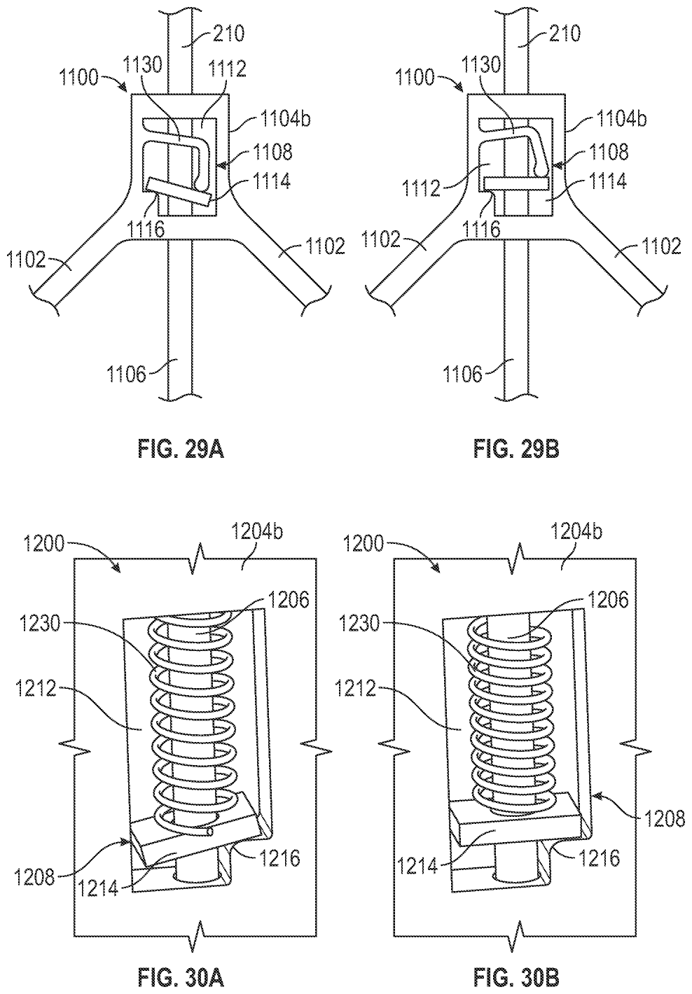

FIGS. 29A-29B depict a portion of a frame 1100 of a prosthetic valve. The frame 1100 can, for example, be configured generally similar to the frame 900 and can function is a substantially similar manner. For example, FIG. 29A depicts the frame 1100 in a locked configuration, and FIG. 29B depicts the frame 1100 in an unlocked configuration. As such, for the sake of brevity, the frame 1100 is labeled with reference numbers that correspond to the components of the frame 900 but increased by 200.

One difference between the frame 1100 and the frame 900 is that the locking mechanism 1108 of the frame 1100 comprises a biasing member 1130. The biasing member 1130 has the form of a pivoting arm. The biasing member 1130 is configured to apply an axially-directed force on one side of the locker disc 1114. Due to the biasing member 1130, the locker disc 1114 contacts the support shoulder 1116 and tilts about the support shoulder to the angled, locked state (FIG. 29A) in a default, resting state. In the angled locked state, the locker disc 1114 engages the actuation member 1106, thereby preventing the actuation member 1106 from moving relative to the second non-pivoting struts 1104b toward the first end of the frame 1100. In this manner, the locking mechanism 1108 can secure the frame 1100 in a desired radially-expanded configuration.

The locking mechanism 1108 can be moved from the angled, locked configuration (FIG. 29A) to the lateral, unlocked configuration (FIG. 29B), for example, by moving the actuation member 1106 toward the second end of the frame 1100. This can be accomplished, for example, by tensioning the actuation shaft 210 of the delivery apparatus with sufficient force to overcome the opposing force of the biasing member 1130. The force can cause the biasing member to pivot within the window 1112 (e.g., upward in the depicted orientation) and the locker disc 1114 to pivot about the support shoulder 1116 from the angled configuration to the lateral configuration. In the lateral configuration, the opening in the locker disc 1114 is sufficiently aligned with the actuation member 906. This releases the actuation member 1106 from the locker disc 1114, allows the actuation member 1106 to move axially relative to the locker disc 1114 toward the second end of the frame 1100, and results in radial expansion of the frame.

When the frame 1100 is expanded to a desired diameter, tension on the actuation shaft 210 of the delivery apparatus and thus on the actuation member 1106 can be reduced such that the force on the locker disc 1114 is less than the opposing force on the locker disc 1114 from the biasing member 1130. The biasing member 1130 can pivot (e.g., upward in the depicted orientation) within the window 1112, and the locker disc 1114 can pivot about the support shoulder 1116 from the lateral configuration to the angled configuration, which secures the frame 1100 at the desired diameter.

In some examples, the biasing member 1130 can have an aperture formed therein such that the actuation member 1106 of the frame and/or an actuation shaft 210 of the delivery apparatus can extend therethrough. In other examples, the biasing member can be formed without an aperture, and the biasing member can be disposed radially inwardly or radially outwardly relative to the actuation member 1106. In yet other examples, a plurality of biasing members can be provided. In such instances, a first biasing member can be disposed radially inwardly relative to the actuation member 1106, and a second biasing member can be disposed radially outwardly relative to the actuation member 1106.

FIGS. 30A-30B depict a portion of a frame 1200 of a prosthetic valve. The frame 1200 can, for example, be configured generally similar to the frame 1100 and can function in a substantially similar manner. For example, FIG. 30A depicts the frame 1200 in a locked configuration, and FIG. 30B depicts the frame 1200 in an unlocked configuration. As such, for the sake of brevity, the frame 1200 is labeled with reference numbers that correspond to the components of the frame 1100 but increased by 100.

One difference between the frame 1200 and the frame 1100 is that the biasing member 1230. The biasing member 1230 is a coil compression spring, whereas the biasing member 1130 is a pivoting arm.

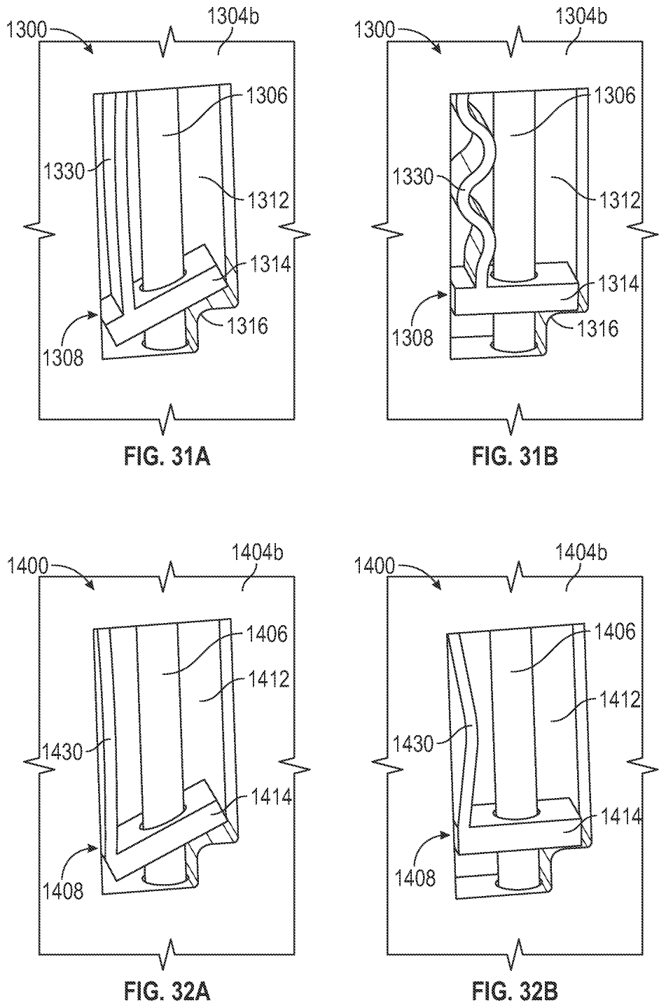

FIGS. 31A-31B depict a portion of a frame 1300 of a prosthetic valve. The frame 1300 can, for example, be configured generally similar to the frame 1100 and can function in a substantially similar manner. For example, FIG. 31A depicts the frame 1300 in a locked configuration, and FIG. 31B depicts the frame 1300 in an unlocked configuration. As such, for the sake of brevity, the frame 1300 is labeled with reference numbers that correspond to the components of the frame 1100 but increased by 200.

One difference between the frame 1300 and the frame 1100 is that the biasing member 1330. The biasing member 1330 is a wave spring, whereas the biasing member 1130 is a pivoting arm.

FIGS. 32A-32B depict a portion of a frame 1400 of a prosthetic valve. The frame 1400 can, for example, be configured generally similar to the frame 1100 and can function in a substantially similar manner. For example, FIG. 32A depicts the frame 1400 in a locked configuration, and FIG. 32B depicts the frame 1400 in an unlocked configuration. As such, for the sake of brevity, the frame 1400 is labeled with reference numbers that correspond to the components of the frame 1100 but increased by 300.

One difference between the frame 1400 and the frame 1100 is that the biasing member 1430. The biasing member 1430 is a leaf spring, whereas the biasing member 1130 is a pivoting arm.

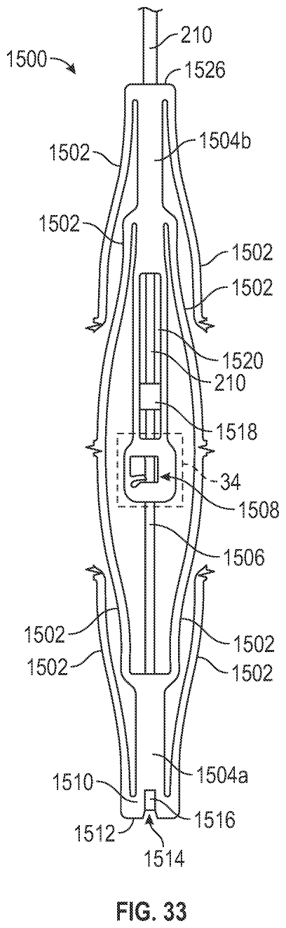

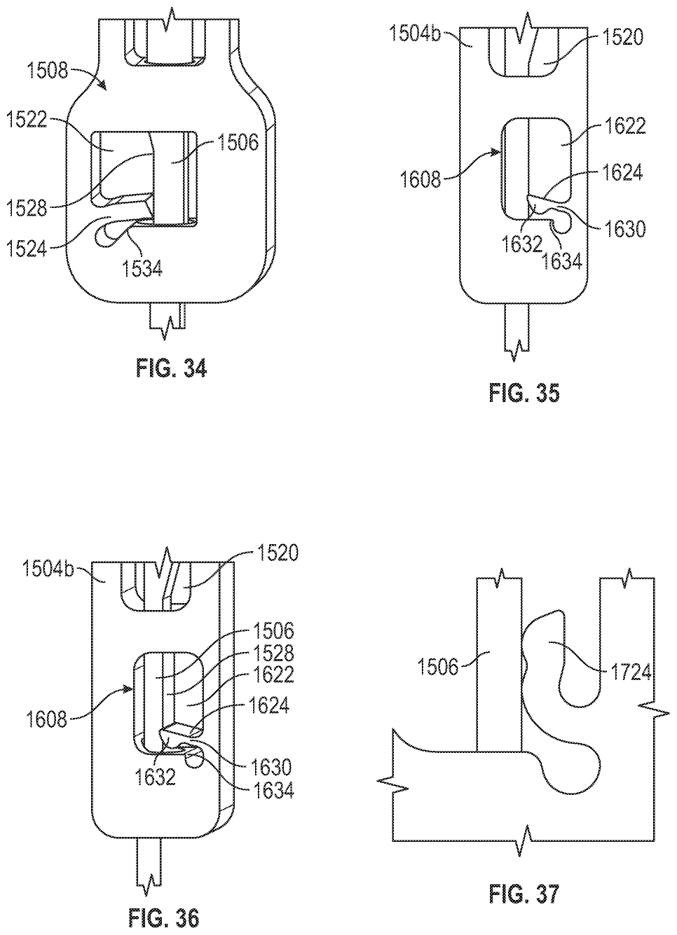

FIGS. 33-34 depict a portion of a frame 1500 for a prosthetic heart valve. The frame 1500 comprises a plurality of pivoting struts 1502, a plurality of non-pivoting struts (including a first non-pivoting strut 1504a and a second non-pivoting strut 1504b, which are collectively or generically referred to as “the non-pivoting struts 1504”), an actuation member 1506 (which can also be referred to as “a pull member”), and a locking mechanism 1508. The frame 1500 is configured similar to the other frames disclosed herein in that the frame 1500 is mechanically expandable to various diameters via moving the actuation member 1506 relative to the locking mechanism 1508 and the second non-pivoting strut 1504b and that the frame 1500 can be locked at a desired diameter via the locking mechanism 1508.

The actuation member 1506 is fixedly coupled to and extends through a lumen of the first non-pivoting strut 1504a and extends from the first non-pivoting strut 1504a toward the second non-pivoting strut 1504b. The actuation member 1506 can be fixedly coupled to the first non-pivoting strut 1504a in various ways. For example, as depicted in FIG. 33, an apex 1510 of the frame 1500 disposed at a first end portion 1512 of the frame 1500 (e.g., an inflow end portion) comprises a notch 1514 formed therein configured to receive one or more tabs 1516 of the actuation member 1506. The tabs 1516 of the actuation member 1506 and the notch 1514 of the frame 1500 can be configured to restrict axial movement in the proximal direction and rotational movement of the actuation member 1506 relative to the frame 1500. The locking mechanism 1508 can restrict axial movement of the actuation member 1506 in the distal direction. The actuation member can be fixedly coupled to the first non-pivoting strut in various other ways, including integrally forming the actuation member and the first non-pivoting strut (e.g., together with the rest of the frame). Alternatively, the actuation member can be fixedly coupled to the first non-pivoting strut via fasteners, welding, adhesive, and/or other means for fixedly coupling.

The actuation member 1506 can also comprise a connection portion disposed at the end opposite the tabs 1516 (i.e., a proximal end portion). The connection portion of the actuation member 1506 can be releasably coupled to the actuation shaft 210 of the delivery apparatus in various ways, including by a nut 1518, other threaded connection, or other means for releasably coupling (e.g., sutures, wires, etc.). In some examples, the nut can be fixedly coupled to the actuation shaft of the delivery apparatus. In other examples, the nut can be fixedly coupled to the actuation member 1506. In the illustrated example, the frame comprises a slot 1520 configured for receiving the nut 1518. In some examples, the portions of the frame defining the slot 1520 can engage the nut 1518 and prevent the nut from rotating relative to the actuation member 1506. In some examples, the nut 1518 can comprise a non-circular cross-sectional profile (e.g., rectangular, hexagonal, etc.) such that the sides or edges of the nut engage the frame adjacent the slot 1520.

Referring to FIG. 34, the locking mechanism 1508 comprises a window 1522 formed in the second non-pivoting strut 1504b (or to a portion of the frame coupled to thereto) and one or more retention elements 1524 disposed in the window 1522 of the frame 1500. The retention element 1524 is configured to engage the actuation member 1506 and to allow the actuation member 1506 to move in a first direction relative to the locking mechanism 1508 (e.g., upward toward a second end portion 1526 (FIG. 33) of the frame in the depicted orientation) and to restrict movement of the actuation member 1506 in a second direction relative to the locking mechanism 1508 (e.g., downward toward the first end portion 1512 (FIG. 33) of the frame in the depicted orientation).

In some examples, the actuation member 1506 can comprise a cylindrical shape, and the retention element 1524 can engage the rounded outside surface of the actuation member 1506. In other examples, the actuation member 1506 can have a surface oriented toward the retention member that is configured to increase frictional engagement between the retention element 1524 and the actuation member 1506. For example, the actuation member 1506 comprises a flat surface 1528 oriented toward the retention element 1524. The flat surface 1528 of the actuation member increases the surface area where the retention element 1524 contacts the actuation member 1506, thereby increasing friction and enhancing locking between the actuation member 1506 and the locking mechanism 1508. Additionally (or alternatively), the actuation member 1506 can comprise projections and/or notches that are configured to engage the retention element, and/or the retention element can comprise projections and/or notches that are configured to engage the actuation member.

The retention element can also be configured in various ways to enhance locking. For example, the retention element can be biased (e.g., via heat setting) in an angled configuration (e.g., non-horizontal in the depicted orientation). In the angled configuration, the free end of the retention element is disposed at least slight farther toward the second end portion 1526 of the frame than the fixed end portion of the retention element.

As another example, FIGS. 35-36 depict a locking mechanism 1608 comprising a window 1622 and one or more retention elements 1624. The locking mechanism 1608 functions similar to the locking mechanism 1508 and can be used, for example, with the frame 1500 in lieu of the locking mechanism 1508.

One difference between the locking mechanism 1608 compared to the locking mechanism 1508 is the shape of the retention element 1624. The retention element 1624 comprise a neck portion 1630, which is relatively thin, and a head portion 1632, which is relative thick. The thin neck portion can, for example, provide increased flexibility of the retention element, allowing it to easily bend proximally as the actuation member moves proximally during frame expansion. The thick head portion can, for example, provide greater strength to the end portion that contacts and is pressed against the actuation member.

Another difference between the locking mechanism 1608 compared to the locking mechanism 1508 is the shape of the window 1622. The window 1622 of the locking mechanism 1608 comprises a shoulder 1634 that is “elevated” (i.e., closer to the second end portion 1526 of the frame) relative a shoulder 1534 (FIG. 34) of the locking mechanism 1508. Elevating the shoulder 1634 of the window 1622 in this manner (and/or the shape of the head portion 1632 of the retention element 1624) can, for example, help to prevent or reduce the likelihood of the retention element 1624 deflecting “downward” past a horizontal plane because the retention element will contact the shoulder 1634 and thus stop deflecting downward while the retention element is still in a slightly upward configuration. Preventing the retention element from moving to a downward configuration can help ensure that the locking mechanism 1508 does not inadvertently move from a locked configuration to an unlocked configuration.

In lieu of or in addition to forming the retention element and/or the shoulder as depicted in FIGS. 35-36, the locking mechanism can also comprise various other features configured to enhance engagement between the retention element and the actuation member and/or to help prevent the retention element from moving to a position in which it is angled downward. For example, FIG. 37 depicts a retention element 1724 that has been shape-set (e.g., via heat setting) in an upward configuration. This upward configuration can, for example, increase the contact area of the retention element 1724 with the actuation member 1506, thereby improving the locking engagement therebetween. The upward configuration of the retention element 1724 can also reduce the likelihood of the retention element moving to a downward configuration, thereby ensuring that the locking mechanism does not inadvertently move from a locked configuration to an unlocked configuration.

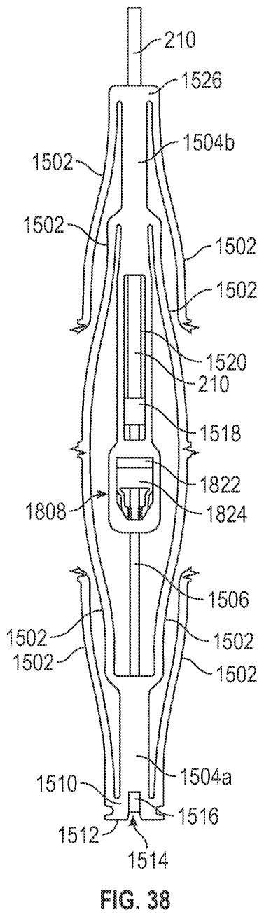

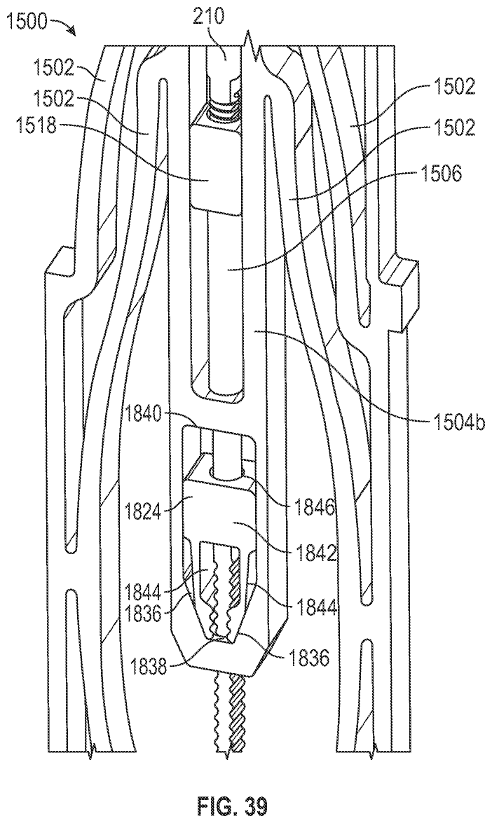

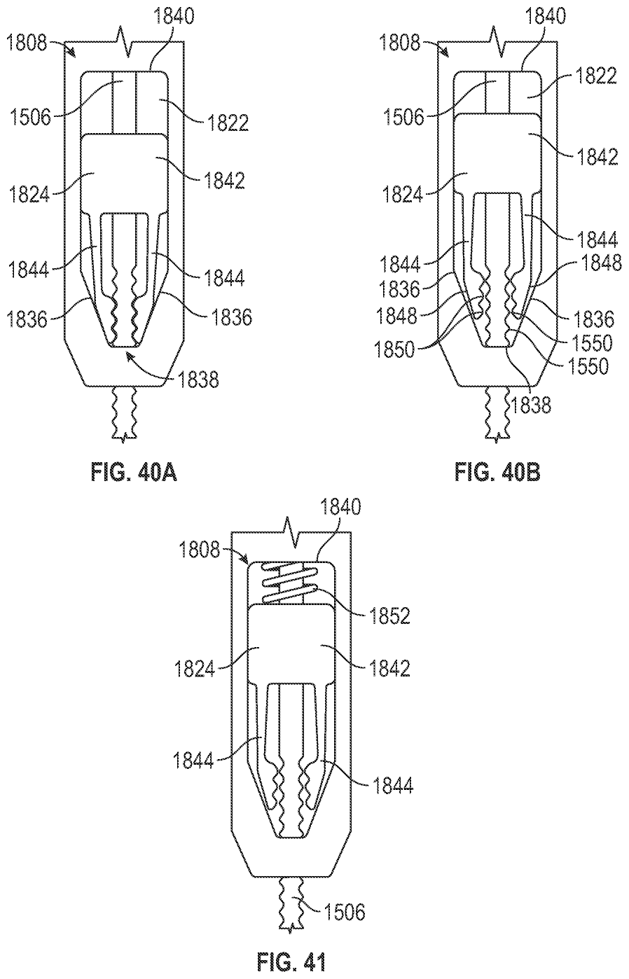

FIGS. 38-41 depict a locking mechanism 1808. As depicted in FIGS. 38-39, the locking mechanism 1808 can be used, for example, with the frame 1500 in place of the locking mechanism 1508. The locking mechanism 1808 comprises a chamber 1822 (which can also be referred to as “a window”) and a retention member 1824 disposed in the chamber 1822. The retention member 1824 can move axially (e.g., up and down in the depicted orientation) within the chamber between a locked position (e.g., FIGS. 38-40A) and an unlocked position (e.g., FIG. 40B-41), as further described below.

The chamber 1822 of the locking mechanism 1808 is formed in and/or coupled to the second non-pivoting strut 1504b, as illustrated in FIGS. 38-39. Referring to FIG. 40A, the chamber 1822 comprises ramped side walls 1836 disposed toward a first end 1838 of the chamber 1822. Stated another way, the chamber 1822 comprises a “V” shape in a plane parallel to the longitudinal axis of the frame. The ramped side walls 136 taper outwardly from a smallest width (e.g., in the horizontal direction in the depicted orientation) at the first end 1838 of the chamber 1822 to a largest width toward a second end 1840 of the chamber 1822. The ramped side walls 1836 are configured to move the retention member 1824 from the unlocked configuration to the locked configuration and to retain the retention member 1824 in the locked configuration.

Referring to FIGS. 40A-40B, the retention member 1824 of the locking mechanism 1808 comprises a base segment 1842 and one or more arms 1844 (e.g., two in the illustrated example) extending from the base segment 1842. The base segment 1842 of the retention member 1824 comprises a lumen 1846 (FIG. 39) configured to receive the actuation member 1506. The arms 1844 of the retention member 1824 are configured to move between a closed state (e.g., FIG. 40A), in which the arms engage the actuation member 1506, and an open state (e.g., FIG. 40B), in which the arms disengage the actuation member 1506. In this manner, the arms 1844 can also be referred to as “jaws” or “clamping elements,” which are movable between an open configuration and a closed configuration. The arms 1844 are biased to the open state in which the clamping force on actuation member 1506 is reduce such that the actuation member 1506 can move proximally relative to the arms 1844, though the arms 1844 can (at least in some instances) maintain at least some contact with the actuation member 1506. The ramped side walls 1836 of the chamber 1822 are configured to move the arms 1844 inward from the open state to the closed state when the arms contact the side walls 1836.

In lieu of or in addition to the arms 1844 maintaining at least some contact with the actuation member 1506, the lumen 1846 of the base segment 1842 can be configured such that the base segment of the retention member 1824 remains engaged with the actuation member 1506. Therefore, the retention member 1824 and the actuation member 1506 move axially together within the chamber 1822. The bias of the frame 1500 to a radially compressed configuration (or a partially radially-expanded configuration) and/or the radially-inward force from the patient's native anatomy pulls actuation member 1506 and the retention member 1824 toward the first end 1838 of the chamber 1822, as depicted in FIG. 40A. In this position, the ramped side walls 1836 of the chamber 1822 contact the arms 1844 and urge the arms 1844 inward against the actuation member 1506. The first end 1838 of the chamber 1822 prevents the retention member 1824 from moving further toward the first end portion 1512 of the frame 1500, and the arms 1844 prevent the actuation member 1506 from moving further toward the first end portion 1512 (FIG. 38) of the frame 1500. In this manner, the locking mechanism 1808 secures the frame 1500 in a desired radially-expanded configuration and prevents the frame from radially compressing.

The locking mechanism 1808 can be moved from the locked configuration to the unlocked configuration (e.g., to radially expand the frame) by moving the actuation member 1506 toward the second end portion 1526 of the frame 1500 (FIG. 38) (e.g., via the actuation shaft 210). The frictional engagement between the base segment 1842 of the retention member 1824 result in the retention member 1824 moving axially together with the actuation member 1506 away from the first end 1838 of the chamber 1822 to the second end 1840 of the chamber 1822 (e.g., upward in the depicted orientation). As the retention member 1824 moves upward within the chamber 1822, the ramped side walls 1836 of the chamber 1822 allow the arms 1844 of the retention member 1824 to move outward and disengage from the actuation member 1506 (though at least some contact can remain), as depicted in FIG. 40B. The base segment 1842 of the retention member 1824 can continue to move upward together with the actuation member 1506 until the base segment 1842 contacts the second end 1840 of the chamber 1822. At this point, the actuation member 1506 can move proximally relative to the lumen 1846 of the retention member 1824 to radially expand the frame 1500.

The locking mechanism 1808 can be moved from the unlocked configuration to the locked configuration by reducing tension on the actuation member (e.g., via the actuation shaft 210) such that the bias of the frame toward the radially-compressed configuration and/or external forces on the frame (e.g., the native anatomy) pulls the actuation member 1506 downward away from the second end 1840 of the chamber 1822 toward the first end 1838 of the chamber 1822. Due to the frictional engagement therebetween, the retention member 1824 moves axially downward together with the actuation member 1506. The arms 1844 of the retention member 1824 contact the ramped side walls 1836 of the chamber 1822, which urges the arms 1844 inwardly against the actuation member 1506, thereby preventing relative movement therebetween. The retention member 1824 and the actuation member 1506 move axially downward together until the arms 1844 of the retention member 1824 contact the first end 1838 of the chamber 1822. At this point, both the retention member 1824 and the actuation member 1506 are restricted from moving further axially downwardly relative to the chamber 1822, which secures the frame 1500 in a particular radially-expanded configuration.

As depicted in FIG. 40B, the arms 1844 of the retention member 1824 can, in some examples, comprise ramped outer surfaces 1848. The ramped outer surfaces 1848 of the arms 1844 can comprise the same or similar angle as the ramped side walls 1836 of the chamber 1822. This can, for example, facilitate relative axial movement between the arms 1844 of the retention member 1824 and the side walls 1836 of the chamber 1822.

Referring still to FIG. 40B, in some instances, inner surfaces of the arms 1844 can comprise teeth 1850 and/or other types of friction-increasing elements configured to engage the actuation member 1506. Additionally (or alternatively), the actuation member 1506 can comprise teeth 1550 and/or other types of friction-increasing elements configured to engage the inner surfaces of the arms 1844 (e.g., the teeth 1850).

Referring to FIG. 41, the locking mechanism 1808 can, in some examples, further comprise a biasing member 1852 (e.g., a compression spring, a wave spring, a leaf spring, etc.) disposed between the base segment 1842 of the retention member 1824 and the second end 1840 of the chamber 1822. The biasing member 1852 can, for example, help bias the locking mechanism 1808 to the locked configuration with the arms 1844 disposed against the first end 1838 of the chamber 1822 (e.g., FIG. 40A). As depicted in FIG. 41, the biasing member 1852 can be configured (e.g., via the spring coefficient) such that the retention member 1824 can still move proximally together with the actuation member 1506, thereby allowing the locking mechanism 1808 to move from the locked configuration to the unlocked configuration. When the retention member 1824 moves proximally toward the second end 1840 of the chamber 1822 (e.g., via the actuation shaft 210 and actuation member 1506), the biasing member 1852 compresses and the retention member moves to the unlocked configuration (e.g., FIG. 41). When the proximal (e.g., upward) force on the retention member 1824 is less than the opposing distal (e.g., downward) force of the biasing member, the retention member 1824 is urged from the unlocked configuration to the locked configuration, which secures the frame in a radially expanded state.

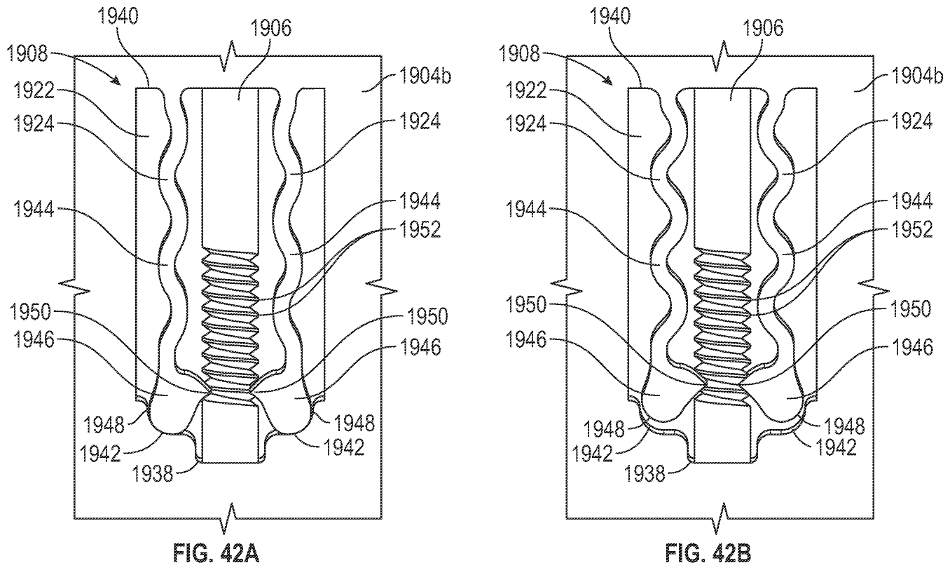

FIGS. 42A-42B depict a locking mechanism 1908. The locking mechanism 1908 can be used, for example, with the frame 1500 in lieu of the locking mechanism 1508. The locking mechanism 1908 comprises a chamber 1922 (which can also be referred to as “a window”) and one or more retention members 1924. In the depicted example, the locking mechanism 1908 comprises two retention members 1924. In other examples, the locking mechanism can have fewer (e.g., one) or more (e.g., 3-4) retention members.

The locking mechanism 1908 is movable between a locked state (FIG. 42A) and an unlocked state (FIG. 42B). In the locked state, the actuation member 1906 is restricted from moving distally (e.g., downwardly in the depicted orientation) relative to the locking mechanism 1908, thereby preventing the frame from radially compressing. In the unlocked state, the actuation member 1906 can move proximally (e.g., upwardly in the depicted orientation) relative to the locking mechanism 1908, thereby allowing the frame to radially expand.

The chamber 1922 of the locking mechanism 1908 can be formed in and/or coupled to a non-pivoting strut of a frame (e.g., a second non-pivoting strut 1904b). The chamber 1922 comprises a first end portion 1938 and a second end portion 1940. The first end portion 1938 of the chamber 1922 comprises one or more curved surfaces 1942 configured to contact respective retention members 1924 and to retain the retention members 1924 against the actuation member 1906, thereby locking the actuation member 1906 relative to the locking mechanism 1908.

Each of the retention members 1924 of the locking mechanism 1908 comprise an arm 1944 (or “arm portion”) and a cam 1946 (or “cam portion”). The arm 1944 comprises a fixed end portion extending from the second end portion 1940 of the chamber 1922 and a free end portion disposed toward the first end portion 1938 of the chamber 1922. The cam 1946 is coupled to the free end portion of the arm 1944.

The arms 1944 of the retention members 1924 are flexible so that the arms can move axially relative to the chamber 1922 in a spring-like manner. For example, FIG. 42A depicts the arms 1944 in a relaxed, uncompressed state, and FIG. 42B depicts the arms 1944 in a deflected, compressed state. In the depicted example, the arms 1944 comprise a wave-spring configuration. In other examples, the arms 1944 can have various other configurations, including coil spring, leaf spring, etc.

The cams 1946 of the retention members 1924 comprise rounded portions 1948 and teeth 1950. The rounded portions 1948 of the cams 1946 are configured to engage the curved surfaces 1942 of the chamber 1922 to retain and/or position the teeth 1950 of the cams 1946 against the actuation member 1906. The teeth 1950 are configured to engage corresponding projections (e.g., threads 1952, teeth, etc.) and/or notches of the actuation member 1906.

In the locked state (FIG. 42A), the teeth 1950 of the retention members 1924 engage the threads 1952 of the actuation member 1906, and the curved surfaces 1942 of the chamber 1922 engage the rounded portions 1948 of the retention members 1924. In this manner, the retention members 1924 and the chamber 1922 prevent the actuation member 1906 from moving distally relative to the locking mechanism 1908, which in turn prevents the frame from radially compressing.

As indicated above, the locking mechanism 1908 can be moved from the locked state (FIG. 42A) to the unlocked state (FIG. 42B). This can be accomplished, for example, by moving the actuation member 1906 proximally relative to the chamber 1922 (e.g., via an actuation shaft of a delivery apparatus that is releasably coupled to the actuation member 1906). The teeth 1950 of the retention members 1924 remain engaged with the threads 1952 of the actuation member 1906 as the actuation member 1906 moves proximally (e.g., upwardly in the depicted orientation). This results in the arms 1944 of the retention members 1924 axially compressing/foreshortening, and the rounded portions 1948 of the cams 1946 moving away from the curved surfaces 1942 of the chamber 1922, as depicted in FIG. 42B. The arms 1944 of the retention members 1924 are biased to remain in contact with the actuation member 1906. Because the cams 1946 disengage from the chamber 1922, the arms 1944 can deflect (elastically) outwardly away from the actuation member 1906, thereby allowing the actuation member 1906 to move axially relative to the teeth 1950 of the retention members 1924. This relative movement can result in radial expansion of the frame. Due to the bias of the teeth 1950 of the retention members 1924, inward against the actuation member 1906, the teeth 1950 “click” along (or slide “over”) the threads in a ratchet-like manner.

When the frame is expanded to a desired state (e.g., a particular diameter), tension on the actuation member 1906 can be reduced such that the bias of the frame to the radially-compressed configuration and/or the native anatomy acting on the frame moves the actuation member 1906 distally relative to the chamber 1922. The retention member 1924 moves distally together with the actuation member 1906. The rounded portion 1948 of the retention member 1924 contacts the curved surfaces 1942 of the chamber 1922, which locks the teeth 1950 of the retention members 1924 against the actuation member 1906 and prevents the retention member 1924 and the actuation member 1906 from moving further distally relative to the chamber 1922. As a result, the frame is prevented from compressing radially.

In some examples, the cams 1946 can pivot (e.g., deflect) at least slightly relative to the arms 1944 as the rounded portion 1948 of the cams 1946 engage/disengage the curved surfaces 1942 of the chamber 1922 when locking/unlocking the locking mechanism 1908. In such examples, the pivot point can be at or adjacent the location where the cams 1946 are coupled to the arms 1944. In some examples, each of the arms 1944 can comprise a hinge element (e.g., a notch) formed therein to facilitate and/or control the pivoting of the cams 1946 relative to the arms 1944.

In examples in which the actuation member 1906 comprises threads 1952 configured to engage with the teeth 1950 of the retention members 1924, the frame can be recompressed from a locked radially-expanded configuration, for example, by rotating the actuation member 1906 relative to the retention members 1924. This allows the actuation member 1906 to move axially as the actuation member 1906 rotates relative to the second non-pivoting strut 1904b as the teeth 1950 of the retention members 1924 traverse the helical path of the threads 1952. The pitch of the threads of the actuation member can be selected to determine the extent of axial movement per rotation of the actuation member (i.e., larger pitch, more axially movement per rotation; smaller pitch, less axial movement per rotation). In this manner, the locking mechanism 1908 comprises “dual actuation,” including “pull-to-expand” actuation and “rotate-to-compress” actuation.

In configurations comprising dual actuation, the actuation member is formed separately from and not fixedly coupled to the frame so that the actuation member can rotate relative to the first non-pivoting strut and is axially fixed relative to the first non-pivoting strut. This can be accomplished, for example, by forming the first non-pivoting strut with a lumen extending therethrough configured for receiving a distal end portion of the actuation member. The actuation member can have radial flanges (e.g., bushings) fixedly coupled thereto (and/or formed thereon) adjacent to the proximal and distal ends of the first non-pivoting strut. The radial flanges can be radially larger than the lumen of the first non-pivoting strut. In this manner, the actuation member can rotate within the lumen of the first non-pivoting strut (e.g., during radial compression of the frame) because the actuation member is at least slightly smaller than the lumen of the first non-pivoting strut, and the actuation member is prevented from moving axially within the lumen of the first non-pivoting strut (e.g., during radial expansion of the frame) because the radial flanges act as stoppers against the proximal and distal ends of the first non-pivoting strut.

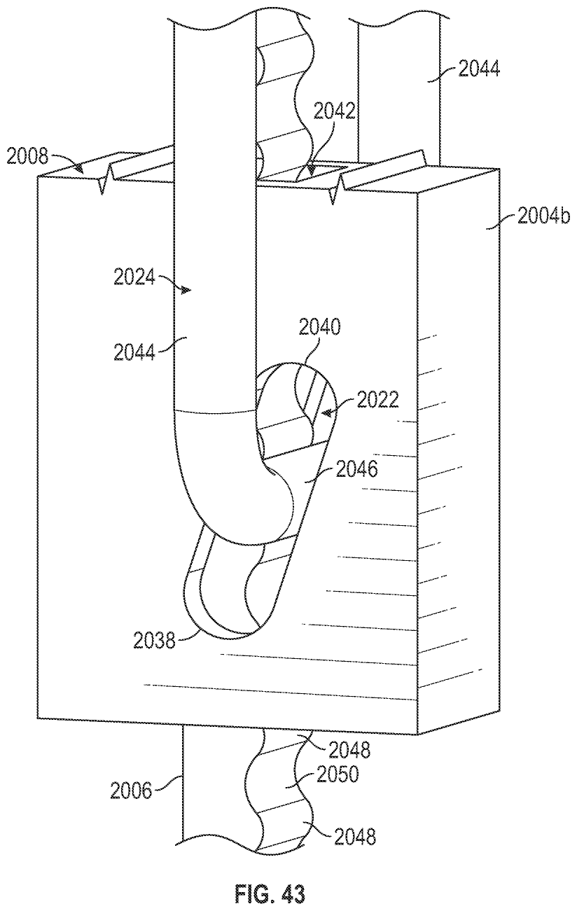

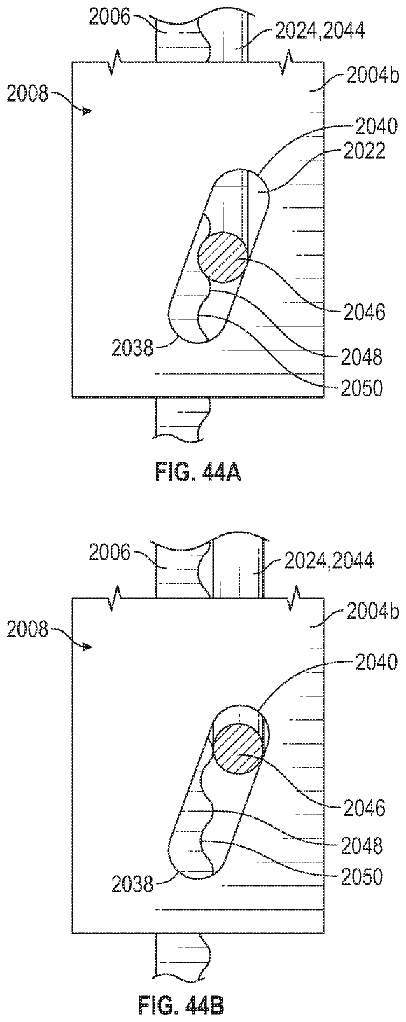

FIGS. 43-44B depict a locking mechanism 2008. The locking mechanism 2008 can be used, for example, with the frame 1500 in lieu of the locking mechanism 1508 and/or adapted for use with one or more of the other frames disclosed herein. The locking mechanism 2008 comprises a chamber 2022 (which can also be referred to as “a window”) and a retention member 2024. In the depicted example, the locking mechanism 2008 comprises one window and one retention member. In other examples, the locking mechanism can have a plurality (e.g., 2-4) of chambers and/or retention members (e.g., such as two chambers spaced axially with one retention member disposed in each of the two chambers).

The locking mechanism 2008 is movable between a locked state (FIGS. 43 and 44A) and an unlocked state (FIG. 44B). In the locked state, the actuation member 2006 is restricted from moving distally (e.g., downwardly in the depicted orientation) relative to the locking mechanism 2008, thereby preventing the frame from radially compressing. In the unlocked state, the actuation member 2006 can move proximally (e.g., upwardly in the depicted orientation) relative to the locking mechanism 2008, thereby allowing the frame to radially expand.

Referring to FIG. 43, the chamber 2022 of the locking mechanism 2008 can be formed in and/or coupled to a non-pivoting strut of a frame (e.g., a second non-pivoting strut 2004b). The chamber 2022 comprises an angled slot having a first end portion 2038 and a second end portion 2040 which is offset laterally relative to the first end portion 2038. The first end portion of the chamber 2022 can intersect with an axially-extending lumen 2042 of the second non-pivoting strut 2004b through which the actuation member 2006 extends. In this manner, the lumen 2042 configured for receiving the actuation member 2006 comprises a first longitudinal axis, and the chamber comprises a second longitudinal axis, which is oblique relative to the first longitudinal axis (see FIG. 44A).

The retention member 2024 of the locking mechanism 2008 can comprise one or more arm portions 2044 and a hook portion 2046. As depicted in FIG. 43, the retention member 2024 comprises two arm portions 2044 coupled to the hook portion 2046. In some examples, the retention member can form a rectangular shape or a “U” shape. In other examples, the retention member can comprise a single arm portion and a hook portion, forming an “L” shape or a “J” shape. The arm portions 2044 of the retention member 2024 can be coupled to the frame at a location proximal to the chamber 2022 (e.g., at or adjacent a second end portion of the frame). The hook portion 2046 of the retention member 2024 are disposed in the chamber 2022, and the retention member 2024 is movable relative to the chamber 2022. Due to the angled orientation of the chamber 2022, the hook portion 2046 of the retention member 2024 moves toward and engages the actuation member 2006 as the hook portion 2046 moves toward the first end portion 2038 of the chamber 2022 (see, e.g., FIG. 44A) and moves away from and disengages the actuation member 2006 as the hook portion 2046 moves toward the second end portion 2040 of the chamber 2022 (see, e.g., FIG. 44B). In some examples, the retention member 2024 is biased toward the first end portion 2038 of the chamber 2022. This can be accomplished in various ways, including forming the arm portions 2044 as springs and/or by coupling one or more springs to the retention member 2024.

The actuation member 2006 can comprise one or more projections 2048 and/or grooves 2050 oriented toward the retention member 2024. The hook portion 2046 of the retention member 2024 can engage the projections 2048 and/or the grooves 2050 of the actuation member 2006 to lock the actuation member 2006.

In the locked position, the hook portion 2046 of the retention member 2024 engages the actuation member 2006, as depicted in FIGS. 43 and 44A. The hook portion 2046 is urged against the side (e.g., the right side in the depicted orientation) of the chamber 2022 and against the actuation member 2006. In this manner, the hook portion 2046 of the retention member 2024 binds the actuation member 2006 and prevents the actuation member 2006 from moving distally (e.g., downwardly in the depicted orientation) relative to the second non-pivoting strut 2004b. This in turn prevents the frame from radially compressing.

When the actuation member 2006 is moved proximally (e.g., upwardly in the depicted orientation) from the position depicted in FIG. 44A, the retention member 2024 moves proximally therewith initially. As the retention member 2024 moves proximally, the hook portion 2046 of the retention member 2024 is guided toward the second end portion 2040 of the chamber 2022 at an angle and at least partially disengages the actuation member 2006, as depicted in FIG. 44B. The actuation member 2006 can therefore be moved further in the proximal direction relative to the hook portion 2046 of the retention member 2024 and the second non-pivoting strut 2004b, which results in radial expansion of the frame.

Once the frame is expanded to a desired configuration, the frame can be locked in the place. The actuation member 2006 and the retention member 2024 are biased toward the distal direction, so reducing tension on the actuation member 2006 (via a delivery apparatus) results in the actuation member 2006 and the retention member 2006 moving distally together. More specifically, the hook portion 2046 of the retention member 2024 engages the projections 2048 and grooves 2050 of the actuation member 2006, and the retention member 2024 and the actuation member 2006 move together in the distal direction until the distance between a groove of the actuation member 2006 in which the hook portion 2046 of the retention member 2024 is disposed and the opposing side wall of the chamber 2022 is equal to the width of the hook portion 2046 of the retention member 2024. At this point, the hook portion 2046 of the retention member 2024 is wedged between the actuation member 2006 and the angled side wall of the chamber 2022 (see FIG. 44A). In this manner, the retention member 2024 binds the actuation member 2006 and prevents the actuation member 2006 from moving further distally, thereby locking the frame in an expanded state and preventing re-compression. The binding occurs because a width (e.g., left-right in the depicted orientation) of the chamber is configured to be less than a combined width of the actuation member and the hook portion of the retention member.

In the example described above, the retention member 2024 may be deemed to be “passively actuated” because it is moved proximally via the actuation member 2006 and distally via the actuation member 2006 and/or the bias of the retention member 2024 to a distal position. In other examples, the retention member can be configured to be “actively actuated.” In such examples, the retention member can be independently actuated (e.g., via a releasable connection with a delivery apparatus (e.g., via a suture releasably coupled to the retention member and extending to a handle of the delivery apparatus)) relative to the actuation member 2006. For example, the locking mechanism can be unlocked by moving the actuation member and the retention member proximally. Once unlocked, the actuation member can be moved further proximally and/or distally while the retention member is disengaged from the actuation member. Thus, the frame can be radially expanded and contracted as desired. The locking mechanism can be locked by moving the retention member distally and back into engagement with the actuation member. This can be accomplished, for example, by releasing the retention member from the proximal position and allowing the bias of the retention member to the distal position to move the retention member distally and/or by moving the retention element distally (e.g., via the delivery apparatus).

It should be noted that the various “locking mechanism” described herein can also be referred to as “a locking member.”

FIGS. 45A-47 depict a delivery assembly comprising the frame 500 and a delivery apparatus 2100, which, for purposes of illustration, are only partially shown. The delivery apparatus 2100 is similar to the delivery apparatus 200 in that it can be releasably coupled to a frame and/or actuation member of a prosthetic heart valve and can be used to radially compress and/or radially expand the prosthetic heart valve. One difference between the delivery apparatus 2100 and the delivery apparatus 200 is that the delivery apparatus 2100 is releasably coupled to the prosthetic heart valve via a plurality of interlocking shafts of an actuation assembly rather than a threaded connection like the delivery apparatus 200.

Referring to FIGS. 45A-45B, the delivery apparatus 2100 comprises an actuation shaft 2102, a locking shaft 2104, and a support sleeve 2106. It should be noted that for simplicity only one set of shafts is depicted (i.e., a set comprising the actuation shaft, the locking shaft, and the support sleeve 2106), but a delivery apparatus can comprise more than one set of shafts/sleeves (e.g., 2-15, including 3-6, 6-12, 6-9, 3, 5, 6, 7, 8, 9, 10, 11, 12, 13, 14, or 15 in particular examples). It should also be noted that the delivery apparatus can further comprise additional components (e.g., a handle, one or more other shafts, etc.) similar to the delivery apparatus 200.

The depicted shafts of the delivery apparatus 2100 are coaxial and axially movable relative to each other. More specifically, the actuation shaft 2102 extends coaxially through the locking shaft 2104 and through the support sleeve 2106, and the locking shaft 2104 extends coaxially through the support sleeve 2106. The shafts and sleeves can be coupled to a handle and/or other shafts of the delivery apparatus at their proximal end portions and releasably coupled to the frame of a prosthetic heart valve (or other prostheses such as stents) at their distal end portions. The actuation shaft 2102 can be used, for example, to apply a proximally-directed force on a distal end portion of the frame 500 to radially expand the frame 500. The locking shaft 2104 can be used, for example, to selectively secure the actuation shaft 2102 to the prosthetic heart valve, as further explained below. The support sleeve 2106 can be used, for example, to apply a distally-directed force on a proximal end portion of the frame 500 to radially expand the frame 500. In this manner, the actuation shaft 2102 and the support sleeve 2106 apply opposing forces to axially compress and radially expand a prosthetic heart valve, and the locking shaft 2104 can be used to secure/release the actuation shaft 2102 from the prosthetic heart valve.

The actuation shaft 2102 of the delivery apparatus 2100 is an elongate, flexible shaft that extends axially from a handle of the delivery apparatus 2100 to the prosthetic heart valve. The actuation shaft 2102 is configured with sufficient rigidity to allow it to move proximally and distally relative to one or more other components of the delivery apparatus (e.g., the handle, the locking shaft 2104, the support sleeve 2106, etc.) and/or the prosthetic valve and sufficient flexibility to allow it to traverse a patient's vasculature. The actuation shaft is also configured to apply a tensile force on the prosthetic valve to move the prosthetic valve from a radially-compressed configuration to a radially-expanded configuration. In some examples, the actuation shaft 2102 can be a tube, rod, cable, wire, suture, etc.

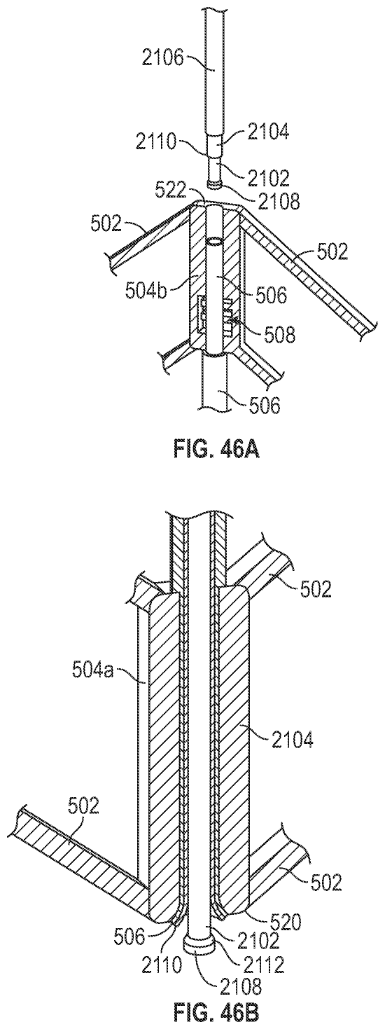

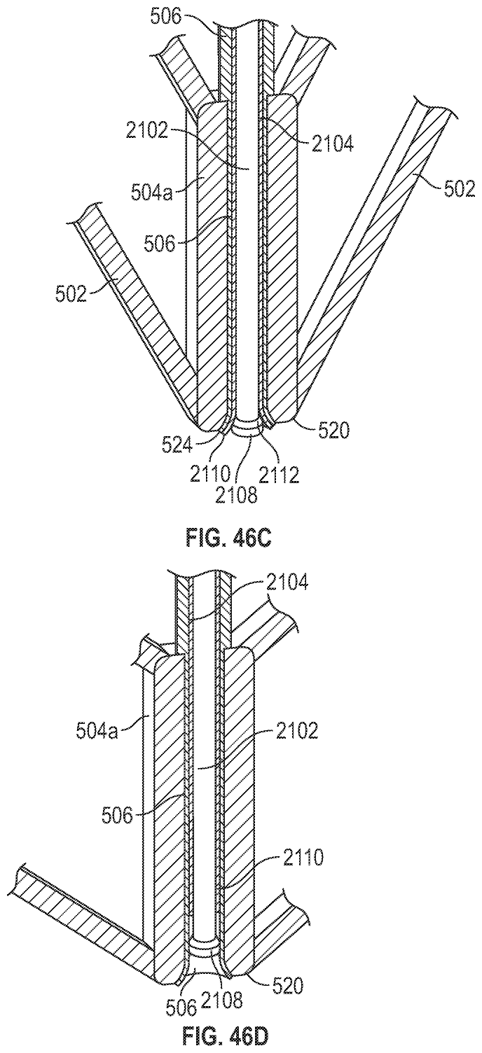

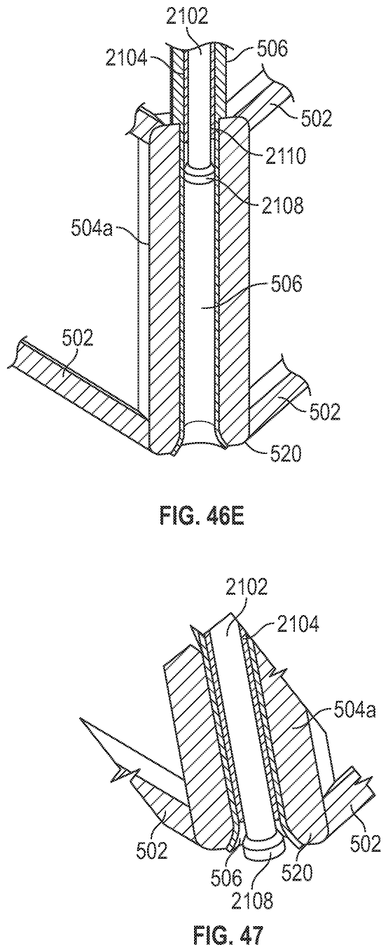

Referring to FIGS. 46A-46B, the actuation shaft 2102 can comprise a stopper 2108 (which may also be referred to as “a flange” or “a flared end portion”) at the distal end. The stopper 2108 extends radially outwardly relative to the portion of the actuation shaft adjacent the stopper. Stated another way, the stopper 2108 comprises a larger diameter than the main portion of the actuation shaft. The diameter (or width) of the stopper 2108 is smaller than the inner diameter of the lumen(s) that extends axially through the non-pivoting struts 504 and the actuation member 506 of the frame 500, as well as the inner diameter of the support sleeve 2106 of the delivery apparatus 2100. The diameter of the stopper 2108 is larger than the inner diameter of the main portion of the locking shaft 2104. As such, the actuation shaft 2102 can move proximally and distally relative to the frame 500 and the support sleeve 2106 (unless the path of the actuation shaft 2102 is obstructed by the locking shaft 2104). The actuation shaft 2102 can also move distally relative to the locking shaft 2104 and proximally relative to the locking shaft 2104 to the point at which the stopper 2108 of the actuation shaft 2102 abuts the distal end portion of the locking shaft 2104. Additional details about the relative movement of the shafts of the delivery apparatus are provided below.

In some examples, the stopper 2108 and the actuation shaft 2102 can be integrally formed as a single, unitary component (e.g., via machining, molding, etc.). In other examples, the stopper 2108 and the actuation shaft 2102 can be formed as separate components that are coupled together (e.g., via welding, fasteners, adhesive, crimping, and/or other means for coupling). For example, in certain examples, the stopper 2108 can be a grommet that is crimped onto (or otherwise coupled to) the distal end portion of the actuation shaft 2102.

The locking shaft 2104 of the delivery apparatus 2100 is an elongate, flexible shaft that extends axially from a handle of the delivery apparatus 2100 to the prosthetic heart valve. The locking shaft 2104 is configured with sufficient rigidity to allow it to move proximally and distally relative to one or more other components of the delivery apparatus (e.g., the handle, the actuation shaft 2102, the support sleeve 2106, etc.) and/or the prosthetic valve and sufficient flexibility to allow it to traverse a patient's vasculature.

The locking shaft 2104 comprises a lumen extending from a proximal end to a distal end of the locking shaft 2104. The lumen is configured such that the actuation shaft 2102 can extend coaxially through the locking shaft 2104 (excluding the stopper 2108 of the actuation shaft 2102).

Referring to FIGS. 46B-46C, the locking shaft 2104 of the delivery apparatus 2100 further comprises a flange portion 2110 disposed at the distal end of the locking shaft 2104. The flange portion 2110 flares radially outwardly and contacts the distal end portion of the actuation member 506 and/or the distal end portion of the frame 500. It should be noted that, although the actuation member 506 and the frame 500 are depicted as separate components in FIGS. 46A-47, the actuation member and the frame can be integrally formed as a single unitary component. In examples comprising a separately formed actuation member, the distal end portion of the actuation member 506 can comprise a flange portion 524, which is similar to the flange portion 2110 of the locking shaft 2104.

The flange portion 2110 of the locking shaft 2104 is configured such that it can be moved between a flared configuration (e.g., FIG. 46C) and a straight configuration (e.g., FIG. 46D). This can be accomplished, for example, by forming the flange portion 2110 of the locking shaft 2104 (and/or one or more other portions of the locking shaft 2104) from an elastically deformable material (e.g., a polymer such as polyimide and/or a metal such as nitinol or stainless steel). The locking shaft 2104 can be configured to be in the flared configuration in a resting or undeflected state. In this manner, the flange portion 2110 of the locking shaft 2104 assumes the flared configuration when it is exposed from the distal end of the lumen of the actuation member 506 and/or the frame 500. Due to its flexibility, the flange portion 2110 can be moved from the flared configuration to the straight configuration by moving the flange portion 2110 proximally into the lumen of the actuation member 506 and/or the frame 500.

A prosthetic device (e.g., the frame 500) can be releasably coupled to the delivery apparatus 2100 using the actuation shaft 2102 and the locking shaft 2104. As one example, referring first to FIG. 46A, the actuation shaft 2102 can be positioned such that the stopper 2108 is disposed distal to the flange portion 2110 of the locking shaft 2104. The distal end portion of the actuation shaft 2102 including the stopper 2108 can be inserted and advanced distally through the lumen of the non-pivoting struts 504 of the frame 500 and/or the actuation member 506 such that the stopper 2108 is disposed distal to the distal end 520 of the frame 500, as depicted in FIG. 46B. The distal end portion of the locking shaft 2104 can then be inserted and advanced distally through the lumen of the non-pivoting struts 504 of the frame 500 and/or the actuation member 506 such that the flange portion 2110 of the locking shaft 2104 is disposed distal to the distal end 520 of the frame 500, which allows the flange portion 2110 of the locking shaft 2104 to flare radially outwardly. In some instances, a temporary sleeve or other restraint can be used to retain the flange portion 2110 of the locking shaft 2104 in the straight configuration while the flange portion 2110 is initially inserted into the lumen of the frame and/or the actuation member. The actuation shaft 2102 can then be moved proximally relative to the frame and the locking shaft 2104 until the stopper 2108 of the actuation shaft 2102 contacts the flange portion 2110 of the locking shaft 2104, as depicted in FIG. 46C. At this point, the stopper 2108 of the actuation shaft 2102 urges the locking shaft 2104 against the frame or actuation member, thereby securing the locking shaft 2104 relative to the frame. The locking shaft 2104 obstructs the lumen of the frame/actuation member, which prevents the actuation shaft 2102 from moving proximally relative to the frame.

As depicted in FIG. 46C, the stopper 2108 of the actuation shaft 2102 can, in some examples, comprise a tapered portion 2112 at the proximal end of the stopper. The tapered portion 2112 can be configured to mate with the flange portion 2110 of the locking shaft 2104, thereby increasing the surface area and thus frictional engagement between the stopper 2108 of the actuation shaft 2102 and the flange portion 2110 of the locking shaft 2104.

As another example, rather than forming the locking shaft with the flange portion prior to inserting the locking shaft through the lumen of a frame, the locking shaft can be initially formed in a straight configuration (i.e., without the flange portion formed). In some such examples, the straight locking shaft can be inserted through the lumen of the frame, and then the distal end portion of the locking shaft can be plastically deformed to form the flange portion. This can be accomplished for example, by pulling the stopper 2108 of the actuation shaft 2102 against the distal end portion of the locking shaft with sufficient force to plastically deform the locking shaft from the straight configuration to a flared configuration. In lieu of and/or prior using the stopper 2108 to form the flange portion, some other deforming device (e.g., a mandrel, a punch, a hammer, etc.) can be used to plastically deform the distal end portion of the locking shaft after it is inserted through the lumen of the frame.

As yet another example, the locking shaft 2104 and the actuation shaft 2102 can be loaded through the prosthetic heart valve distally to proximally (i.e., opposite from the proximal-to-distal method described above). This method can be used, for example, when the proximal end portions of the actuation shaft and the locking shaft are not yet coupled to (or are released from) the handle of the delivery apparatus. In such examples, the proximal end the locking shaft can be advanced proximally through the lumen of the non-pivoting struts of the frame and/or the actuation member until the flange portion 2110 of the locking shaft abuts the distal end portion of the frame 500 and/or the actuation member, as depicted in FIG. 46B. The proximal end of the actuation shaft 2102 can then be inserted and advanced proximally in a similar manner until the stopper 2108 of the actuation shaft contacts the flange portion 2110 of the locking shaft 2104, as depicted in FIG. 46C. At this point, the actuation shaft 2102 compresses the locking shaft 2104 between the stopper and the frame or actuation member, thereby securing the locking shaft relative to the frame, and the locking shaft 2104 prevents the actuation shaft 2102 from further proximal movement relative to the frame 500.

Regardless of the method of initially assembling, with the actuation shaft 2102 secured to the frame 500 via the locking shaft 2104 (e.g., FIG. 46C), the actuation shaft 2102 can be used to radially expand the frame. This can be accomplished, for example, by tensioning the actuation shaft 2102, which applies a proximally-directed force on the distal end 520 of the frame 500. The support sleeve 2106 of the delivery apparatus can contact (e.g., abut) the proximal end 522 of the frame 500 and apply an opposing distally-directed force thereto. The axially-compressive force on the frame 500 via the actuation shaft 2102 and the support sleeve 2106 results in radial expansion of the frame (see, e.g., FIG. 45A-45B). The frame 500 can be expanded to a desired configuration and secured in the desired configuration via the engagement between locking mechanism 508 and the actuation member 506 of the frame 500, which is depicted in FIG. 46A and further described above.

The frame 500 can be released from the delivery apparatus 2100 in the following manner. The actuation shaft 2102 of the delivery apparatus 2100 can be advanced distally relative to the locking shaft 2104, thereby removing the clamping force on the flange portion 2110 of the locking shaft 2104. In some examples, the stopper 2108 of the actuation shaft 2102 can be spaced distally apart from the flange portion 2110 of the locking shaft 2104, as depicted, for example, in FIG. 46B. In other examples, the stopper 2108 is not spaced apart from the flange portion, but the tension on the actuation shaft 2102 is reduced such that the locking shaft 2104 can be moved proximally relative to the actuation shaft 2102, as depicted, for example, in FIG. 47. In either case, the locking shaft 2104 is released from the actuation shaft 2102 such that the flange portion 2110 of the locking shaft 2104 can be drawn into the lumen of the frame and moved from the flared configuration to the straight configuration, as depicted in FIGS. 46D-47. The locking shaft 2104 and the actuation shaft 2102 can then be moved further proximally (see FIG. 46E) and withdrawn from the lumen of the frame, thereby releasing the frame from the delivery apparatus (see, e.g., FIG. 46A). In some examples, the locking shaft 2104 can be retracted proximally first, followed by the actuation shaft 2102. In other examples, the locking shaft 2104 and the actuation shaft 2102 can be retracted proximally simultaneously.

The delivery apparatus 2100 advantageously allows a prosthetic device to be coupled and released in a relatively simple manner (e.g., relatively axial movement between the actuation shaft 2102 and the locking shaft 2104). The delivery apparatus 2100 is also relatively robust and/or easy to manufacture because it is does not rely on a threaded connection, which can be challenging for components having small dimensions.

The delivery apparatus 2100 can be used with any of the prosthetic heart valves or frames disclosed herein. The delivery apparatus 2100 can also be used with various other prosthetic devices (e.g., stents, grafts, etc.) that are releasably coupled to a delivery apparatus.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve comprising a frame that includes a plurality of struts, an actuation member, a locking mechanism, a first end portion, and a second end portion. The frame is movable from a radially-compressed configuration and a plurality of radially-expanded configurations. The plurality of struts and the locking mechanism are integrally formed as a unitary component. The actuation member extends from the first end portion of the frame toward the second end portion of the frame. The locking mechanism is disposed at the second end portion of the frame and is configured to receive the actuation member. The locking mechanism is configured to selectively engage the actuation member such that the actuation member is movable in a first direction relative to the frame when the frame is in a first radially-expanded configuration, thereby allowing further radial expansion of the frame from the first radially-expanded configuration to a second radially-expanded configuration, and such that the actuation member is prevented from moving in a second direction relative to the frame when the frame is in the first radially-expanded configuration, thereby preventing the frame from moving from the first radially-expanded configuration to the radially-compressed configuration.

Example 2. The prosthetic heart valve of any example herein, particularly example 1, further comprising a valve structure coupled to the frame, wherein the valve structure comprises a plurality of leaflets configured for allowing blood to flow in an antegrade direction and to restrict blood from flowing in a retrograde direction.

Example 3. The prosthetic heart valve of any example herein, particularly any one of examples 1-2, wherein the actuation member of the frame comprises a first threaded portion configured for threadably coupling the actuation member to a second threaded portion of a delivery apparatus.

Example 4. The prosthetic heart valve of any example herein, particularly any one of examples 1-2, wherein the frame comprises a lumen configured for receiving a plurality of shafts of a delivery apparatus configured for releasably coupling the prosthetic heart valve to the delivery apparatus via a non-threaded connection.

Example 5. The prosthetic heart valve of any example herein, particularly any one of examples 1-4, wherein the actuation member, the plurality of struts, and the locking mechanism are integrally formed as a unitary component.

Example 6. The prosthetic heart valve of any example herein, particularly any one of examples 1-5, wherein the plurality of struts of the frame includes a plurality of pivoting struts and a plurality of non-pivoting struts, wherein the pivoting struts are integrally formed with or fixedly coupled to the non-pivoting struts, and wherein the pivoting struts pivot relative to the non-pivoting struts as the frame moves from the radially-compressed configuration to the first radially-expanded configuration.

Example 7. The prosthetic heart valve of any example herein, particularly example 6, wherein the actuation member is coupled to a first non-pivoting strut of the plurality of non-pivoting struts, which is disposed at the first end portion of the frame, and wherein the locking mechanism is formed in a second non-pivoting strut of the plurality of non-pivoting struts, which is disposed at the second end portion of the frame.

Example 8. The prosthetic heart valve of any example herein, particularly any one of examples 1-7, wherein the actuation member comprises an elongate shaft.

Example 9. The prosthetic heart valve of any example herein, particularly any one of examples 1-7, wherein the actuation member comprises an elongate rod.

Example 10. The prosthetic heart valve of any example herein, particularly any one of examples 1-9, wherein the actuation member comprises a circular cross-sectional profile taken in a plane perpendicular to a longitudinal axis of the actuation member.

Example 11. The prosthetic heart valve of any example herein, particularly any one of examples 1-9, wherein the actuation member comprises a non-circular cross-sectional profile taken in a plane perpendicular to a longitudinal axis of the actuation member.

Example 12. The prosthetic heart valve of any example herein, particularly any one of examples 1-9, wherein the actuation member comprises a rectangular cross-sectional profile taken in the plane perpendicular to the longitudinal axis of the actuation member.

Example 13. The prosthetic heart valve of any example herein, particularly any one of examples 1-12, wherein the actuation member comprises one or more projections extending outwardly therefrom and configured to engage the locking mechanism.

Example 14. The prosthetic heart valve of any example herein, particularly any one of examples 1-13, wherein the actuation member comprises one or more notches formed therein and configured to receive the locking mechanism.

Example 15. The prosthetic heart valve of any example herein, particularly any one of examples 1-14, wherein the locking mechanism comprises one or more tabs configured to frictionally engage the actuation member.

Example 16. The prosthetic heart valve of any example herein, particularly any one of examples 1-14, wherein the locking mechanism comprises one or more tongues configured to frictionally-engage the actuation member.

Example 17. The prosthetic heart valve of any example herein, particularly any one of examples 1-14, wherein the locking mechanism comprises one or more arms configured to frictionally-engage the actuation member.

Example 18. The prosthetic heart valve of any example herein, particularly any one of examples 1-17, wherein the locking mechanism is biased to a locked position and is movable from the locked position to an unlocked position by moving the actuation member relative to the second end portion of the frame.

Example 19. The prosthetic heart valve of any example herein, particularly example 18, wherein the locking mechanism is biased to the locked position by shape-setting the locking mechanism in the locked position.

Example 20. The prosthetic heart valve of any example herein, particularly example 18 or example 19, wherein the locking mechanism is biased to the locked position by a spring.

Example 21. A prosthetic heart valve comprising a frame including a plurality of struts, a plurality of actuation members, a plurality of locking mechanisms, a first end portion and a second end portion. The frame is movable from a radially-compressed configuration to a plurality of radially-expanded configurations. The plurality of struts and the plurality of locking mechanisms are integrally formed as a unitary component. The plurality of actuation members extends from the first end portion of the frame toward the second end portion of the frame, each actuation member of the plurality of actuation members being spaced circumferentially relative to an adjacent actuation member of the plurality of actuation members. The plurality of locking mechanisms is disposed at the second end portion of the frame, each locking mechanism of the plurality of locking mechanisms being spaced circumferentially relative to an adjacent locking mechanism of the plurality of actuation members and configured to receive a respective actuation member of the plurality of actuation members and to selectively engage the respective actuation member such that the respective actuation member is movable in a first direction relative to the frame when the frame is in the first radially-expanded configuration, thereby allowing further radial expansion of the frame from the first radially-expanded configuration to a second radially-expanded configuration, and such that the respective actuation member is prevented from moving in a second direction relative to the frame when the frame is in the first radially-expanded configuration, thereby preventing the frame from moving from the first radially-expanded configuration to the radially-compressed configuration.

Example 22. The prosthetic heart valve of any example herein, particularly example 21, wherein the plurality of actuation members comprises 2-15 actuation members, and wherein the plurality of locking mechanisms comprises 2-15 locking mechanisms.

Example 23. The prosthetic heart valve of any example herein, particularly example 21, wherein the plurality of actuation members comprises 3-12 actuation members, and wherein the plurality of locking mechanisms comprises 3-12 locking mechanisms.

Example 24. The prosthetic heart valve of any example herein, particularly example 21, wherein the plurality of actuation members comprises 6-9 actuation members, and wherein the plurality of locking mechanisms comprises 6-9 locking mechanisms.

Example 25. The prosthetic heart valve of any example herein, particularly example 21, wherein the plurality of actuation members comprises exactly three actuation members, and wherein the plurality of locking mechanisms comprises exactly three locking mechanisms.

Example 26. The prosthetic heart valve of any example herein, particularly example 21, wherein the plurality of actuation members comprises exactly six actuation members, and wherein the plurality of locking mechanisms comprises exactly six locking mechanisms.

Example 27. The prosthetic heart valve of any example herein, particularly example 21, wherein the plurality of actuation members comprises exactly nine actuation members, and wherein the plurality of locking mechanisms comprises exactly nine locking mechanisms.

Example 28. The prosthetic heart valve of any example herein, particularly example 21, wherein the plurality of actuation members comprises exactly twelve actuation members, and wherein the plurality of locking mechanisms comprises exactly twelve locking mechanisms.

Example 29. The prosthetic heart valve of any example herein, particularly any one of examples 21-28, wherein the plurality of actuation members, the plurality of struts, and the plurality of locking mechanisms are integrally formed as a unitary component.

Example 30. The prosthetic heart valve of any example herein, particularly any one of examples 21-28, wherein the plurality of actuation members is formed as separate components from the plurality of struts and the plurality of locking mechanisms and is fixedly coupled to the frame.

Example 31. A method of implanting a prosthetic heart valve, comprising inserting a prosthetic heart valve into a patient's vasculature, the prosthetic heart valve releasably coupled to a distal end portion of a delivery apparatus and in a radially-compressed configuration. The method further comprises advancing the prosthetic heart valve through the patient's vasculature to an implantation location, expanding the prosthetic heart valve to a radially-expanded configuration by applying an axially-compressive force on the prosthetic heart valve with the delivery apparatus, and locking the prosthetic heart valve in the radially-expanded configuration by engaging an actuation member and a locking mechanism of the prosthetic heart valve such that the prosthetic heart valve is prevented from moving from the radially-expanded configuration to the radially-compressed configuration, wherein the locking mechanism is integrally formed with a frame of the prosthetic heart valve.

Example 32. The method of any example herein, particularly example 31, wherein the actuation member is integrally formed with the frame and the locking mechanism.

Example 33. The method of any example herein, particularly any one of examples 31-32, wherein prior to expanding the prosthetic heart valve to the radially-expanded configuration by applying the axially-compressive force on the prosthetic heart valve with the delivery apparatus, the method further comprises deploying the prosthetic heart valve from a delivery capsule of the delivery apparatus, and allowing the prosthetic heart valve to self-expand from the radially-compressed configuration to a partially-radially expanded configuration, which is less than the radially-expanded configuration.

Example 34. The method of any example herein, particularly any one of examples 31-33, wherein after locking the prosthetic heart valve in the radially-expanded configuration, the method further comprises releasing the prosthetic heart valve from the delivery apparatus by rotating a shaft of the delivery apparatus relative to the actuation member of the prosthetic heart valve.

Example 35. The method of any example herein, particularly any one of examples 31-33, wherein after locking the prosthetic heart valve in the radially-expanded configuration, the method further comprises releasing the prosthetic heart valve from the delivery apparatus by moving an inner shaft of the delivery apparatus distally relative to an outer shaft of the delivery apparatus followed by moving the inner shaft and the outer shaft proximally relative to the prosthetic heart valve.

Example 36. A prosthetic heart valve comprising a frame and a valve structure. The frame comprising a plurality of struts, an actuation member, a locking mechanism, a first end portion and a second end portion. The frame is movable from a radially-compressed configuration and a radially-expanded configuration. The plurality of struts and the locking mechanism are integrally formed as a unitary component. The actuation member extends from the first end portion of the frame toward the second end portion of the frame. The locking mechanism is disposed at the second end portion of the frame and comprises a channel and a retention element. The channel is configured to receive the actuation member. The retention element is configured to selectively engage the actuation member such that the actuation member is movable in a first direction relative to the channel as the frame moves from the radially-compressed configuration to the radially-expanded configuration and such that the actuation member is prevented from moving in a second direction relative to the channel when the frame is in the radially-expanded configuration, thereby preventing the frame from moving from the radially-expanded configuration to the radially-compressed configuration. The valve structure coupled to the frame and comprising a plurality of leaflets configured for allowing blood to flow in an antegrade direction and to restrict blood from flowing in a retrograde direction.

Example 37. The prosthetic heart valve of any example herein, particularly example 36, wherein the locking mechanism further comprises a window defined by a first side wall, a second side wall, a first end wall, and a second end wall, wherein the channel extends across the window from the first end wall to the second end wall, and wherein the retention element extends from the first side wall of the window and partially obstructs the channel.

Example 38. The prosthetic heart valve of any example herein, particularly example 37, wherein the retention element comprises a fixed end portion and a free end portion, wherein the fixed end portion extends from the first side wall, and wherein the retention element is angled such that the free end portion is disposed farther toward the first end wall than the fixed end portion.

Example 39. The prosthetic heart valve of any example herein, particularly example 38, wherein an angle between the free end portion of the retention element and the first side wall is within a range of 10-80 degrees.

Example 40. The prosthetic heart valve of any example herein, particularly example 38, wherein an angle between the free end portion of the retention element and the first side wall is within a range of 20-60 degrees.

Example 41. The prosthetic heart valve of any example herein, particularly example 38, wherein an angle between the free end portion of the locking mechanism and the first sidewall is within a range of 30-50 degrees.

Example 42. The prosthetic heart valve of any example herein, particularly any one of examples 36-41, wherein the first end portion of the frame is an inflow end portion, and wherein the second end portion is an outflow end portion.

Example 43. The prosthetic heart valve of any example herein, particularly any one of examples 36-41, wherein the first end portion of the frame is an outflow end portion, and wherein the second end portion is an inflow end portion.

Example 44. The prosthetic heart valve of any example herein, particularly any one of examples 36-43, wherein the actuation member is integrally formed as a unitary component with the plurality of struts and the locking mechanism.

Example 45. The prosthetic heart valve of any example herein, particularly any one of examples 36-44, wherein the actuation member comprises a threaded portion configured for releasably coupling the actuation member to an actuation mechanism of a delivery apparatus.

Example 46. The prosthetic heart valve of any example herein, particularly any one of examples 36-44, wherein the actuation member comprises a lumen configured for releasably coupling the actuation member to an actuation mechanism of a delivery apparatus.

Example 47. The prosthetic heart valve of any example herein, particularly any one of examples 36-46, wherein the actuation member comprises a circular cross-sectional profile taken in a plane perpendicular to a longitudinal axis of the actuation member.

Example 48. The prosthetic heart valve of any example herein, particularly any one of examples 36-46, wherein the actuation member comprises a rectangular cross-sectional profile taken in a plane perpendicular to a longitudinal axis of the actuation member.

Example 49. The prosthetic heart valve of any example herein, particularly any one of examples 36-46, wherein the actuation member comprises a flat side surface facing the locking mechanism, and wherein the locking mechanism is configured to engage the flat side surface of the actuation member.

Example 50. The prosthetic heart valve of any example herein, particularly any one of examples 36-49, wherein the actuation member comprises one or more friction-increasing elements, and wherein the locking mechanism is configured to engage the one or more friction-increasing elements.

Example 51. The prosthetic heart valve of any example herein, particularly example 50, wherein the one or more friction-increasing elements comprise teeth.

Example 52. The prosthetic heart valve of any example herein, particularly any one of examples 50-51, wherein the one or more friction-increasing elements comprise grooves.

Example 53. The prosthetic heart valve of any example herein, particularly any one of examples 50-52, wherein the one or more friction-increasing elements comprise notches.

Example 54. The prosthetic heart valve of any example herein, particularly any one of examples 36-53, wherein the frame is formed of a shape-memory material.

Example 55. The prosthetic heart valve of any example herein, particularly example 54, wherein the frame is formed of nitinol.

Example 56. The prosthetic heart valve of any example herein, particularly any one of examples 36-55, wherein the plurality of leaflets is coupled together to form commissures, and wherein the frame comprises a plurality of commissure attachment posts configured for receiving the commissures of the plurality of the leaflets.

Example 57. The prosthetic heart valve of any example herein, particularly example 56, wherein each commissure attachment post of the plurality of commissure attachment posts comprises a slot configured for receiving a respective commissure.

Example 58. The prosthetic heart valve of any example herein, particularly any one of examples 56-57, wherein the plurality of commissure attachment posts is spaced apart from an outflow end of the frame.

Example 59. The prosthetic heart valve of any example herein, particularly any one of examples 36-58, wherein the plurality of struts forms a plurality of closed cells.

Example 60. The prosthetic heart valve of any example herein, particularly example 59, wherein the plurality of closed cells comprises a plurality of inner cells and a plurality of outer cells.

Example 61. The prosthetic heart valve of any example herein, particularly example 60, wherein each inner cell is disposed within a respective outer cell.

Example 62. The prosthetic heart valve of any example herein, particularly any one of examples 60-61, wherein each inner cell is circumferentially aligned with a respective outer cell.

Example 63. The prosthetic heart valve of any example herein, particularly any one of examples 60-62, wherein the inner cells and outer cells comprise the same shape.

Example 64. The prosthetic heart valve of any example herein, particularly any one of examples 60-63, wherein the inner cells and outer cells comprise a hexagonal shape when viewed in a direction parallel to a longitudinal axis extending from the first end portion of the frame to the second end portion of the frame.

Example 65. The prosthetic heart valve of any example herein, particularly any one of examples 59-63, wherein each of the closed cells comprises a double wishbone shape when viewed in a direction parallel to a longitudinal axis extending from the first end portion of the frame to the second end portion of the frame.

Example 66. The prosthetic heart valve of any example herein, particularly any one of examples 36-65, wherein the actuation member is axially spaced apart from the locking mechanism when the frame is in the radially-compressed configuration.

Example 67. The prosthetic heart valve of any example herein, particularly any one of examples 36-66, wherein the plurality of struts is fixedly coupled together such that a first portion of the plurality of struts deflects relative to a second portion of the plurality of struts when the frame moves from the radially-compressed configuration to the radially-expanded configuration.

Example 68. The prosthetic heart valve of any example herein, particularly example 67, wherein the second portion of the plurality of struts is oriented parallel to a longitudinal axis of the frame when the frame is in the radially-compressed configuration and the radially-expanded configuration, and wherein the first portion of the plurality of struts is oblique relative to the second portion of the plurality of struts when the frame is in the radially-compressed configuration and the radially-expanded configuration.

Example 69. The prosthetic heart valve of any example herein, particularly any one of examples 36-68, wherein the actuation member is one of a plurality of actuation members, and wherein each of the plurality of actuation members is spaced circumferentially relative to an adjacent actuation member.

Example 70. The prosthetic heart valve of any example herein, particularly any one of examples 36-69, wherein the locking mechanism is one of a plurality of locking mechanisms, and wherein each of the plurality of locking mechanisms is spaced circumferentially relative to an adjacent locking mechanism.

Example 71. The prosthetic heart valve of any example herein, particularly any one of examples 36-70, wherein the retention element is one of a plurality of retention elements, and wherein each of the plurality of retention elements is spaced axially relative to an adjacent retention member.

Example 72. The prosthetic heart valve of any example herein, particularly any one of examples 36-71, wherein the radially-expanded configuration is a first radially-expanded configuration, wherein the frame is movable from the radially-compressed configuration to the first radially-expanded configuration and from the first radially-expanded configuration to the a second radially-expanded configuration, and wherein the first radially-expandable configuration is radially larger than the radially-compressed configuration and smaller than the second radially-expanded configuration.

Example 73. The prosthetic heart valve of any example herein, particularly any one of examples 36-72, wherein the frame is movable from the radially-expanded configuration to the radially-compressed configuration.

Example 74. The prosthetic heart valve of any example herein, particularly any one of examples 72-73, wherein the frame formed of a shape memory material such that the frame self-expands from the radially-compressed configuration to the first radially-expanded configuration.

Example 75. The prosthetic heart valve of any example herein, particularly any one of examples 72-74, wherein the frame is movable from the first radially-expanded configuration to a third radially-expanded configuration, wherein the second radially-expanded configuration is larger than the first radially-expanded configuration and the second radially-expanded configuration.

Example 76. The prosthetic heart valve of any example herein, particularly example 75, wherein the frame is movable from the third radially-expanded configuration to the radially-compressed configuration.

Example 77. The prosthetic heart valve of any example herein, particularly any one of examples 36-76, wherein the radially-expanded configuration is a first radially-expanded configuration of a plurality of radially-expanded configurations, wherein the frame is movable from the first radially-expanded configuration to a radially-expanded configuration of the plurality of radially-expanded configurations, wherein the frame comprises a larger diameter in the second radially-expanded configuration than the first radially-expanded configuration, and wherein the locking mechanism prevents the frame from moving from the second radially-expanded configuration to the first radially-expanded configuration.

Example 78. The prosthetic heart valve of any example herein, particularly any one of examples 36-77, wherein the retention element is initially formed in a lateral configuration prior to formation of the channel, wherein the retention element is deflected to an angled configuration while the channel is formed, and wherein the retention element is returned to the lateral configuration after the formation of the channel, wherein the retention element intersects with the channel in the lateral configuration, and wherein the retention element does not intersect with the channel in the angled configuration.

Example 79. The prosthetic heart valve of any example herein, particularly any one of examples 36-77, wherein the retention element is initially formed in an angled configuration prior to formation of the channel, wherein the retention element is in the angled configuration during the formation of the channel, wherein the retention element is moved from the angled configuration to a lateral configuration after the formation of the channel and is shape set in the lateral configuration after the formation of the channel, wherein the retention element does not intersect with the channel in the angled configuration, and wherein the retention element intersects with the channel in the lateral configuration.

Example 80. The prosthetic heart valve of any example herein, particularly any one of examples 36-77, wherein the plurality of struts of the frame comprises a plurality of pivoting struts and a plurality of non-pivoting struts, wherein the pivoting struts are integrally formed with the non-pivoting struts, and wherein the pivoting struts pivot relative to the non-pivoting struts as the frame moves from the radially-compressed configuration to the radially-expanded configuration.

Example 81. A method of implanting a prosthetic heart valve, comprising inserting a prosthetic heart valve into a patient's vasculature, the prosthetic heart valve releasably coupled to a distal end portion of a delivery apparatus and in a radially-compressed configuration, advancing the prosthetic heart valve through the patient's vasculature to an implantation location, expanding the prosthetic heart valve to a radially-expanded configuration by applying an axially-compressive force on the prosthetic heart valve with the delivery apparatus, and locking the prosthetic heart valve in the radially-expanded configuration by engaging an actuation member and a locking mechanism of the prosthetic heart valve such that the prosthetic heart valve is prevented from moving from the radially-expanded configuration to the radially-compressed configuration, wherein the locking mechanism is integrally formed with a frame of the prosthetic heart valve.

Example 82. The method of any example herein, particularly example 81, wherein the actuation member is integrally formed with the frame and the locking mechanism.

Example 83. The method of any example herein, particularly example 81, wherein the actuation member is fixedly coupled to the frame and the locking mechanism.

Example 84. The method of any example herein, particularly example 81, wherein the actuation member is coupled to the frame such that the actuation member is axially fixed and rotatable relative to the frame.

Example 85. The method of any example herein, particularly any one of examples 81-84, wherein prior to expanding the prosthetic heart valve to the radially-expanded configuration by applying the axially-compressive force on the prosthetic heart valve with the delivery apparatus. The method further comprises deploying the prosthetic heart valve from a delivery capsule of the delivery apparatus, and allowing the prosthetic heart valve to self-expand from the radially-compressed configuration to a partially-radially expanded configuration, which is less than the radially-expanded configuration.

Example 86. The method of any example herein, particularly any one of examples 81-85, wherein after locking the prosthetic heart valve in the radially-expanded configuration, the method further comprises releasing the prosthetic heart valve from the delivery apparatus by rotating a shaft of the delivery apparatus relative to the actuation member of the prosthetic heart valve.

Example 87. The method of any example herein, particularly any one of examples 81-85, wherein after locking the prosthetic heart valve in the radially-expanded configuration, the method further comprises releasing the prosthetic heart valve from the delivery apparatus by moving an inner shaft of the delivery apparatus distally relative to an outer shaft of the delivery apparatus followed by moving the inner shaft and the outer shaft proximally relative to the prosthetic heart valve.

Example 88. A prosthetic heart valve comprising a frame including a plurality of struts, an actuation member, a locking mechanism, a first end portion, and a second end portion. The frame is movable from a radially-compressed configuration to a plurality of radially-expanded configurations, which are radially larger than the radially-compressed configuration. The plurality of struts and the locking mechanism are integrally formed as a unitary component. The actuation member extends from the first end portion of the frame toward the second end portion of the frame. The locking mechanism is disposed at the second end portion of the frame and comprises a lumen and a retention element. The lumen is configured to receive the actuation member. The retention element comprises an aperture configured to receive the actuation member. The retention element is biased to a first configuration and is movable between the first configuration and a second configuration. When the retention element is in the first configuration, the aperture of the retention element is misaligned with the actuation member such that one or more portions of the retention element defining the aperture engage the actuation member, thereby preventing the actuation member from moving in a first direction relative to the retention element and securing the frame in one of the radially-expanded configurations. When the retention element is in the second configuration, the aperture of the retention element is aligned with the actuation member, thereby allowing the actuation member to move in a second direction relative to the retention element and allowing the frame to move from the radially-compressed configuration to the plurality of radially-expanded configurations.

Example 89. The prosthetic heart valve of any example herein, particularly example 88, wherein the actuation member comprises a first central longitudinal axis, wherein the aperture of the retention element comprises a second central longitudinal axis, wherein the second central longitudinal axis of the retention element is oblique to the first central longitudinal axis of the actuation member when the retention element is in the first configuration, and wherein the second central longitudinal axis of the retention element is parallel or at least substantially parallel to the first central longitudinal axis of the actuation member when the retention element is in the second configuration.

Example 90. The prosthetic heart valve of any example herein, particularly example 88, wherein the actuation member comprises a first central longitudinal axis, wherein the aperture of the retention element comprises a second central longitudinal axis, wherein an angle between the second central longitudinal axis of the retention element and the first central longitudinal axis of the actuation member is within a range of 5-55 degrees when the retention element is in the first configuration, and wherein the angle between the second central longitudinal axis of the retention element and the first central longitudinal axis of the actuation member is within a range of 0-3 degrees when the retention element is in the second configuration.

Example 91. A prosthetic heart valve comprising a frame with a plurality of struts, an actuation member, a locking mechanism, a first end portion and a second end portion. The frame is movable from a radially-compressed configuration and a radially-expanded configuration, which is radially larger than the radially-compressed configuration. The plurality of struts and the locking mechanism are integrally formed as a unitary component. The locking mechanism is disposed at the first end portion of the frame and comprises a slot, a first retention element, and a second retention element. The first retention element extends from a first side of the slot. The second retention element extends from a second side of the slot. In the radially-expanded configuration, the actuation member is disposed in the slot, the first retention element engages a first segment of the actuation member, and the second retention element engages a second segment of the actuation member, thereby preventing the frame from moving from the radially-expanded configuration to the radially-compressed configuration.

Example 92. The prosthetic heart valve of any example herein, particularly example 91, wherein the first retention element and the second retention element are tabs.

Example 93. The prosthetic heart valve of any example herein, particularly any one of examples 91-92, wherein the first segment of the actuation member is a first side of the actuation member, and wherein the second segment of the actuation member is a second side of the actuation member, the second side being opposite the first side.

Example 94. The prosthetic heart valve of any example herein, particularly any one of examples 91-92, wherein the actuation member is a cylindrical shaft, and wherein the first segment of the actuation member is diametrically opposed to the second segment of the actuation member.

Example 95. The prosthetic heart valve of any example herein, particularly any one of examples 91-94, wherein the first retention element is one of a plurality of first retention elements.

Example 96. The prosthetic heart valve of any example herein, particularly any one of examples 91-95, wherein the second retention element is one of a plurality of second retention elements.

Example 97. The prosthetic heart valve of any example herein, particularly any one of examples 91-96, wherein the first retention element is axially aligned with the second retention element.

Example 98. The prosthetic heart valve of any example herein, particularly any one of examples 91-96, wherein the first retention element is axially offset with the second retention element.

Example 99. The prosthetic heart valve of any example herein, particularly any one of examples 91-98, wherein the first end portion of the frame is an inflow end portion, and wherein the second end portion of the frame is an outflow end portion.

Example 100. The prosthetic heart valve of any example herein, particularly any one of examples 91-98, wherein the first end portion of the frame is an outflow end portion, and wherein the second end portion of the frame is an inflow end portion.

Example 101. The prosthetic heart valve of any example herein, particularly any one of examples 91-100, wherein the actuation member comprising a lumen configured for receiving an actuation shaft of a delivery apparatus.

Example 102. The prosthetic heart valve of any example herein, particularly any one of examples 91-101, wherein the locking mechanism is formed in a first non-pivoting strut of the plurality of struts.

Example 103. The prosthetic heart valve of any example herein, particularly example 102, wherein the first non-pivoting strut comprises a threaded bore configured for threadably coupling the frame to an actuation shaft of a delivery apparatus.

Example 104. The prosthetic heart valve of any example herein, particularly example 102, wherein the first non-pivoting strut comprises a bore configured for receiving a locking shaft and an actuation shaft of a delivery apparatus.

Example 105. The prosthetic heart valve of any example herein, particularly any one of examples 91-104, wherein the locking mechanism has a locked configuration corresponding to when the first retention element engages the first segment of the actuation member and the second retention element engage the second segment of the actuation member and the frame is prevented from moving from the radially-expanded configuration to the radially-compressed configuration, wherein the radially-expanded configuration is a first radially-expanded configuration of a plurality of radially-expanded configuration, and wherein the locking mechanism is configured such that when the locking mechanism is in the locked configuration, the frame can be radially expanded from the first radially-expanded configuration to a second radially-expanded configuration.

Example 106. A method of implanting a prosthetic heart valve, comprising inserting a prosthetic heart valve into a patient's vasculature, the prosthetic heart valve releasably coupled to a distal end portion of a delivery apparatus and in a radially-compressed configuration, advancing the prosthetic heart valve through the patient's vasculature to an implantation location, expanding the prosthetic heart valve to a radially-expanded configuration by applying an axially-compressive force on the prosthetic heart valve with the delivery apparatus, and locking the prosthetic heart valve in the radially-expanded configuration by engaging an actuation member and a locking mechanism of the prosthetic heart valve such that the prosthetic heart valve is prevented from moving from the radially-expanded configuration to the radially-compressed configuration, wherein the locking mechanism is integrally formed with a frame of the prosthetic heart valve and comprises a first retention element contacting a first segment of the actuation member and a second retention element contacting a second segment of the actuation member.

Example 107. The method of any example herein, particularly example 106, wherein the first segment of the actuation member is a first side of the actuation member, and wherein the second segment of the actuation member is a second side of the actuation member, which is opposite the first side.

Example 108. The method of any example herein, particularly example 106 or example 107, wherein expanding the prosthetic heart valve to the radially-expanded configuration includes moving the actuation member distally relative to the locking mechanism.

Example 109. The method of any example herein, particularly example 106 or example 107, wherein expanding the prosthetic heart valve to the radially-expanded configuration includes moving the actuation member proximally relative to the locking mechanism.

Example 110. A prosthetic heart valve comprising a frame with a plurality of struts, an actuation member, a locking mechanism, a first end portion, and a second end portion. The frame is movable from a radially-compressed configuration to a plurality of radially-expanded configurations, which are radially larger than the radially-compressed configuration. The plurality of struts and the locking mechanism are integrally formed as a unitary component. The actuation member extends from the first end portion of the frame toward the second end portion of the frame. The locking mechanism is disposed at the second end portion of the frame and extends toward the first end portion of the frame, the locking mechanism including a first aperture and a second aperture axially spaced apart from each other, and each configured to receive the actuation member. The locking mechanism is biased to a first configuration and is movable between the first configuration and a second configuration. When the locking mechanism is in the first configuration, the first aperture and the second aperture of the locking mechanism are misaligned with the actuation member such that one or more portions of the locking mechanism defining the first aperture and the second aperture engage the actuation member, thereby preventing the actuation member from moving in a first direction relative to the locking mechanism and securing the frame in one of the radially-expanded configurations. When the locking mechanism is in the second configuration, the first aperture and the second aperture of the locking mechanism are aligned with the actuation member, thereby allowing the actuation member to move in a second direction relative to the locking mechanism and allowing the frame to move from the radially-compressed configuration to the plurality of radially-expanded configurations.

Example 111. The prosthetic heart valve of any example herein, particularly example 110, wherein the locking mechanism is an elongate tab having a first end extending from the second end portion of the frame, a second end disposed toward the first end portion of the frame relative to the first end, and an intermediate portion disposed between the first end and the second end, and wherein the first aperture and the second aperture are formed in the intermediate portion.

Example 112. The prosthetic heart valve of any example herein, particularly example 111, wherein the first end and the second end of the locking mechanism are disposed on a first side of the actuation member, and the intermediate portion of the locking mechanism is disposed on a second side of the actuation member, the second side being opposite the first side.

Example 113. The prosthetic heart valve of any example herein, particularly any one of examples 110-112, wherein the locking mechanism comprise a wave shape.

Example 114. The prosthetic heart valve of any example herein, particularly any one of examples 110-112, wherein the locking mechanism comprise a “C” shape.

Example 115. The prosthetic heart valve of any example herein, particularly any one of examples 110-114, wherein the locking mechanism comprises a radius of curvature between the first aperture and the second aperture, wherein the radius of curvature is tighter when the locking mechanism is in the second configuration than when the locking mechanism is in the first configuration.

Example 116. The prosthetic heart valve of any example herein, particularly any one of examples 110-115, wherein the first aperture and the second aperture of the locking mechanism are axially spaced apart by a first distance when the locking mechanism is in the first configuration, wherein the first aperture and the second aperture of the locking mechanism are axially spaced apart by a second distance when the locking mechanism is in the second configuration, and wherein the second distance is less than the first distance.

Example 117. The prosthetic heart valve of any example herein, particularly any one of examples 110-116, wherein the first aperture and the second aperture of the locking mechanism are configured such that a plurality of portions of the locking mechanism defining each of the first aperture and the second aperture engage the actuation member when the locking mechanism is in the first configuration.

Example 118. The prosthetic heart valve of any example herein, particularly any one of examples 117, wherein the locking mechanism engages two sides of the actuation member at each of the first aperture and the second aperture.

Example 119. The prosthetic heart valve of any example herein, particularly any one of examples 110-118, wherein the actuation member comprises a cylindrical shape, and wherein each of the first aperture and the second aperture comprises a circular shape.

Example 120. The prosthetic heart valve of any example herein, particularly any one of examples 110-118, wherein the actuation member comprises a non-cylindrical shape, and wherein each of the first aperture and the second aperture comprises a non-circular shape.

Example 121. The prosthetic heart valve of any example herein, particularly example 120, wherein the actuation member comprises a rectangular shape, and wherein each of the first aperture and the second aperture comprises a rectangular shape.

Example 122. The prosthetic heart valve of any example herein, particularly example 121, wherein the actuation member comprises a square shape, and wherein each of the first aperture and the second aperture comprises a square shape.

Example 123. A prosthetic heart valve comprising a frame comprising a plurality of struts, an actuation member, a first locking mechanism, a second locking mechanism, a first end portion, and a second end portion. The frame is movable from a radially-compressed configuration to a plurality of radially-expanded configurations, which are radially larger than the radially-compressed configuration. The plurality of struts, the first locking mechanism, and the second locking mechanism are integrally formed as a unitary component. The actuation member extends from the first end portion of the frame toward the second end portion of the frame. The first locking mechanism and the second locking mechanism are disposed at the second end portion of the frame and extend toward the first end portion of the frame, the first locking mechanism including a first aperture, the second locking mechanism including a second aperture, the first aperture and the second aperture axially spaced apart from each other and configured to receive the actuation member. The first locking mechanism and the second locking mechanism are biased to a first configuration and are movable between the first configuration and a second configuration. When the first locking mechanism and the second locking mechanism are in the first configuration, the first aperture and the second aperture are misaligned with the actuation member such that the first locking mechanism and the second locking mechanism engage the actuation member, thereby preventing the actuation member from moving in a first direction relative to the first locking mechanism and the second locking mechanism and securing the frame in one of the radially-expanded configurations. When the first locking mechanism and the second locking mechanism are in the second configuration, the first aperture and the second aperture are aligned with the actuation member, thereby allowing the actuation member to move in a second direction relative to the first locking mechanism and the second locking mechanism and allowing the frame to move from the radially-compressed configuration to the plurality of radially-expanded configurations.

Example 124. The prosthetic heart valve of any example herein, particularly example 123, wherein each of the first locking mechanism and the second locking mechanism comprise an extension portion and an engagement portion, each extension portion coupled to a non-pivoting strut of the plurality of struts disposed at the second end portion of the frame, and each engagement portion extending from a respective engagement portion and having a respective aperture formed therein.

Example 125. The prosthetic heart valve of any example herein, particularly example 124, wherein the engagement portion of each locking mechanism pivots relative to a respective extension portion when the first locking mechanism and the second locking mechanism move between the first configuration and the second configuration.

Example 126. The prosthetic heart valve of any example herein, particularly example 125, wherein the engagement portions of the first locking mechanism and the second locking mechanism are oblique relative to each other when the first locking mechanism and the second locking mechanism are in the first configuration, and wherein the engagement portions of the first locking mechanism and the second locking mechanism are parallel relative to each other when the first locking mechanism and the second locking mechanism are in the second configuration.

Example 127. The prosthetic heart valve of any example herein, particularly example 125 or example 126, wherein each engagement portion is oblique relative to its respective extension member when the first locking mechanism and the second locking mechanism are in the first configuration, and wherein each engagement portion is perpendicular relative to its respective extension member when the first locking mechanism and the second locking mechanism are in the second configuration.

Example 128. The prosthetic heart valve of any example herein, particularly any one of examples 124-127, wherein the extension portion of the first locking mechanism is disposed on a first side of the actuation member, and the engagement portion of the first locking mechanism extends towards a second side of the actuation member, and wherein the extension portion of the second locking mechanism is disposed on the second side of the actuation member, and the engagement portion of the second locking mechanism extends towards the first side of the actuation member.

Example 129. The prosthetic heart valve of any example herein, particularly any one of examples 124-128, wherein the extension portion of the first locking mechanism is longer than the extension portion of the second locking mechanism.

Example 130. The prosthetic heart valve of any example herein, particularly any one of examples 123-129, wherein the first locking mechanism is longer than the second locking mechanism.

Example 131. A prosthetic heart valve comprising a frame including a plurality of struts, an actuation member, a locking mechanism, a first end portion, and a second end portion. The frame is movable from a radially-compressed configuration to a plurality of radially-expanded configurations, which are radially larger than the radially-compressed configuration. The actuation member extends from the first end portion of the frame toward the second end portion of the frame. The locking mechanism comprises a window and a locker disc, the window formed in a non-pivoting strut of the plurality of struts disposed at the second end portion of the frame and comprising a support shoulder, and the locker disc comprising a first side portion, a second side portion, and an opening, the first side portion of the locker disc disposed on the support shoulder, the second side portion spaced from the support shoulder, and the opening configured for receiving the actuation member. The locker disc is pivotable about the support shoulder between a locked position and an unlocked position. When the locker disc is in the locked position, the opening of the locker disc is misaligned with the actuation member and the locker disc engages the actuation member such that the actuation member is prevented from moving in a first direction relative to the window, thereby securing the frame in one of the radially-expanded configurations. When the locker disc is in the unlocked position, the opening of the locker disc is aligned with the actuation member such that the actuation member can move in a second direction relative to the window, thereby allowing the frame to move from the radially-compressed configuration to the plurality of radially-expanded configurations.

Example 132. The prosthetic heart valve of any example herein, particularly example 131, wherein the locker disc comprises a first axis extending from the first side portion to the second side portion and bisecting the opening, and wherein an angle between the first axis of the locker disc and a central longitudinal axis of the actuation member is within a range of 5-85 degrees when the locker disc is in the locked position.

Example 133. The prosthetic heart valve of any example herein, particularly example 131 or example 132, wherein the locker disc comprises a first axis extending from the first side portion to the second side portion and bisecting the opening, and wherein an angle between the first axis of the locker disc and a central longitudinal axis of the actuation member is within a range of 86-90 degrees when the locker disc is in the unlocked position.

Example 134. The prosthetic heart valve of any example herein, particularly example 132 or example 133, wherein the opening of the locker disc comprises an ovular shape taken in a plane perpendicular to the central longitudinal axis of the actuation member, wherein a major axis of the ovular-shaped opening aligns with the first axis of the locker disc, wherein the locker disc engages the actuation member along the major axis when the locker disc is in the locked position and disengages the actuation member along the major axis when the locker disc is in the unlocked position, and wherein the locker disc engages the actuation member along a minor axis of the ovular-shaped opening when the locker disc in the locked position and in the unlocked position.

Example 135. The prosthetic heart valve of any example herein, particularly example 132 or example 133, wherein the locker disc comprises a second axis extending perpendicular to the first axis and bisecting the opening, wherein the locker disc has a curved shape in a plane normal to the second axis.

Example 136. The prosthetic heart valve of any example herein, particularly example 135, wherein the locker disc engages the actuation member along the first axis when the locker disc is in the locked position and disengages the actuation member along the first axis when the locker disc is in the unlocked position, and wherein the locker disc engages the actuation member along the second axis when the locker disc in the locked position and in the unlocked position.

Example 137. The prosthetic heart valve of any example herein, particularly example 135 or example 136, wherein the opening of the locker disc is formed in the locker disc when the locker disc is in a flat configuration, and wherein the locker disc is shape set to the curved shape after the opening is formed.

Example 138. The prosthetic heart valve of any example herein, particularly any one of examples 131-137, wherein the locker disc is removably coupled to the frame via the actuation member.

Example 139. The prosthetic heart valve of any example herein, particularly any one of examples 131-138, wherein the window of the locking mechanism comprises a proximal end wall and a distal end wall, wherein the first end portion of the locker disc contacts the proximal end wall when the locker disc is in the locked position and the unlocked position, and wherein the second end portion of the locker disc is spaced from the proximal end wall when the locker disc is in the locked position and contacts the proximal end wall when the locker disc is in the unlocked position.

Example 140. The prosthetic heart valve of any example herein, particularly example 139, wherein the second end portion of the locker disc is spaced from the distal end wall when the locker disc is in the locked position and the unlocked position.

Example 141. The prosthetic heart valve of any example herein, particularly any one of examples 131-138, wherein the locking mechanism further comprises a biasing member disposed within the window and configured to contact the locker disc and to bias the locker disc to the locked position.

Example 142. The prosthetic heart valve of any example herein, particularly example 141, wherein the biasing member comprises a compression spring disposed between the locker disc and a proximal end wall defining a portion of the window.

Example 143. The prosthetic heart valve of any example herein, particularly example 141, wherein the biasing member comprises a leaf spring disposed between the locker disc and a proximal end wall defining a portion of the window.

Example 144. The prosthetic heart valve of any example herein, particularly example 141, wherein the biasing member comprises a wave spring disposed between the locker disc and a proximal end wall defining a portion of the window.

Example 145. The prosthetic heart valve of any example herein, particularly example 141, wherein the biasing member comprises a pivoting arm disposed between the locker disc and a side wall defining a portion of the window.

Example 146. The prosthetic heart valve of any example herein, particularly any one of examples 141-145, wherein the biasing member circumscribes the actuation member.

Example 147. The prosthetic heart valve of any example herein, particularly any one of examples 141-146, wherein the biasing member comprises an opening configured for receiving the actuation member.

Example 148. The prosthetic heart valve of any example herein, particularly any one of examples 141-145, wherein the actuation member is disposed radially inwardly relative to the biasing member.

Example 149. The prosthetic heart valve of any example herein, particularly any one of examples 141-145, wherein the actuation member is disposed radially outwardly relative to the biasing member.

Example 150. The prosthetic heart valve of any example herein, particularly any one of examples 141-149, the biasing member is integrally formed with the locker disc.

Example 151. The prosthetic heart valve of any example herein, particularly any one of examples 141-149, the biasing member is integrally formed with the locker disc and the non-pivoting strut in which the window is formed.

Example 152. The prosthetic heart valve of any example herein, particularly any one of examples 141-151, wherein the biasing member is a first biasing member of a plurality of biasing members disposed within the window and configured to contact the locker disc and to bias the locker disc to the locked position.

Example 153. A prosthetic heart valve comprising a frame comprising a plurality of struts, an actuation member, a locking mechanism, a first end portion, and a second end portion. The frame is movable from a radially-compressed configuration to a plurality of radially-expanded configurations, which are radially larger than the radially-compressed configuration. The actuation member extends from the first end portion of the frame toward the second end portion of the frame. The locking mechanism comprises a window and a retention element, the window formed in a non-pivoting strut of the plurality of struts disposed at the second end portion of the frame and comprising a shoulder, the retention element disposed in the window and configured to engage the actuation member such that the actuation member is movable in a first direction relative to the retention element and prevented from moving in a second direction relative to the retention element, the first direction corresponding to radial expansion of the frame, and the second direction corresponding to radial compression of the frame.

Example 154. The prosthetic heart valve of any example herein, particularly example 153, wherein the retention element comprises a fixed end portion and a free end portion, wherein the fixed end portion extends from the non-pivoting strut in which the window is formed, and wherein the free end portion is configured to engage the actuation member.

Example 155. The prosthetic heart valve of any example herein, particularly example 154, wherein the fixed end portion of the retention element comprises a first thickness, and wherein the free end portion of the retention element comprises a second thickness, which is greater than the first thickness.

Example 156. The prosthetic heart valve of any example herein, particularly example 154 or example 155, wherein the retention element is angled such that the fixed end portion of the retention element is disposed closer to the first end portion of the frame than the free end portion of the retention element.

Example 157. The prosthetic heart valve of any example herein, particularly any one of examples 154-156, wherein the shoulder is configured to stop the free end portion of the retention element from moving past the shoulder toward the first end portion of the frame.

Example 158. The prosthetic heart valve of any example herein, particularly any one of examples 154-157, wherein the free end portion of the retention element is parallel or substantially parallel to the actuation member.

Example 159. The prosthetic heart valve of any example herein, particularly any one of examples 154-158, wherein the fixed end portion of the retention element is perpendicular or substantially perpendicular to the actuation member.

Example 160. The prosthetic heart valve of any example herein, particularly any one of examples 153-159, wherein the retention element is initially formed in a first configuration and shape set to a second configuration.

Example 161. The prosthetic heart valve of any example herein, particularly any one of examples 153-160, wherein the actuation member comprises a non-cylindrical shape taken in plane perpendicular to a longitudinal axis of the actuation member.

Example 162. The prosthetic heart valve of any example herein, particularly example 161, wherein the non-cylindrical shape includes a flat side direct toward the retention element, and wherein the retention element is configured to engage the flat side.

Example 163. The prosthetic heart valve of any example herein, particularly any one of examples 153-162, wherein the actuation member comprises threads configured for threadably coupling the actuation member to an actuation shaft of a delivery apparatus.

Example 164. The prosthetic heart valve of any example herein, particularly example 163, wherein the frame comprises a slot spaced axially from the window toward the second end portion of the frame, wherein the slot is configured for receiving the actuation member, a nut, and the actuation shaft of the delivery apparatus.

Example 165. The prosthetic heart valve of any example herein, particularly example 164, wherein the nut is fixedly coupled to the actuation member.

Example 166. The prosthetic heart valve of any example herein, particularly example 164, wherein the nut is fixedly coupled to the actuation shaft of the delivery apparatus.

Example 167. A prosthetic heart valve comprising a frame with a plurality of struts, an actuation member, a locking mechanism, a first end portion, and a second end portion. The frame is movable from a radially-compressed configuration to a plurality of radially-expanded configurations, which are radially larger than the radially-compressed configuration. The actuation member extends from the first end portion of the frame toward the second end portion of the frame. The locking mechanism comprises a chamber and a retention member, the chamber formed in a non-pivoting strut of the plurality of struts disposed at the second end portion of the frame and at least partially defined by one or more ramped side walls of the non-pivoting strut, the retention member disposed in the chamber and comprising a base segment and one or more arms extending from the base segment, the base segment comprising a lumen configured for receiving the actuation member, and the one or more arms configured to engage the actuation member. The retention member is axially movable within the chamber between a locked position and an unlocked position. In the locked position, the one or more arms of the retention member contact the one or more ramped side walls of the non-pivoting strut, thereby securing the one or more arms of the retention member against the actuation member and preventing the actuation member from moving axially relative to the retention member toward the first end portion of the frame. In the unlocked position, the actuation member is released from the one or more arms of the retention member such that the actuation member is axially movable relative to the retention member toward the second end portion of the frame.

Example 168. The prosthetic heart valve of any example herein, particularly example 167, wherein the one or more arms of the retention member move laterally relative to the actuation member when the retention member moves between the locked position to the unlocked position.

Example 169. The prosthetic heart valve of any example herein, particularly example 167 or example 168, wherein the one or more arms of the retention member are spaced laterally from the actuation member such that there is a gap therebetween.

Example 170. The prosthetic heart valve of any example herein, particularly any one of examples 167-169, wherein the one or more arms of the retention member are in a closed configuration when the retention member is in the locked position, wherein the one or more arms of the retention member are in an open configuration when the retention member is in the unlocked position, and wherein the one or more arms are biased in the open configuration.

Example 171. The prosthetic heart valve of any example herein, particularly any one of examples 167-170, wherein the lumen of the base segment is sized such that the base segment of the retention member contacts the actuation member when the retention member is in the locked position and the unlocked position.

Example 172. The prosthetic heart valve of any example herein, particularly any one of examples 167-171, wherein the retention member is biased to the locked position.

Example 173. The prosthetic heart valve of any example herein, particularly example 172, wherein the retention member is biased to the locked position via a bias of the frame to the radially-compressed configuration.

Example 174. The prosthetic heart valve of any example herein, particularly example 172 or example 173, wherein the retention member is biased to the locked position via a spring.

Example 175. The prosthetic heart valve of any example herein, particularly any one of examples 167-174, wherein the one or more arms of the retention member comprise teeth configured to engage the actuation member.

Example 176. The prosthetic heart valve of any example herein, particularly any one of examples 167-174, wherein the actuation member comprises teeth configured to engage the actuation member.

Example 177. A prosthetic heart valve comprising a frame having a plurality of struts, an actuation member, a locking mechanism, a first end portion, and a second end portion. The frame is movable from a radially-compressed configuration to a plurality of radially-expanded configurations, which are radially larger than the radially-compressed configuration. The actuation member extends from the first end portion of the frame toward the second end portion of the frame. The locking mechanism comprises a chamber and one or more retention members, the chamber formed in a non-pivoting strut of the plurality of struts disposed at the second end portion of the frame and at least partially defined by one or more curved surfaces of the non-pivoting strut, the one or more retention members disposed in the chamber, each of the one or more retention members comprising an arm portion and a cam portion, the arm portion fixedly coupled to the non-pivoting strut in which the chamber is formed and the cam portion extending from the arm portion and configured to engage the actuation member. The one or more retention members are axially movable within the chamber between a locked position and an unlocked position. In the locked position, the cam portion of each of the one or more retention members contacts a respective curved surface of the non-pivoting strut, which secures the cam portion of each of the one or more retention members against the actuation member and restricts the actuation member from moving axially relative to the one or more retention members toward the first end portion of the frame. In the unlocked position, the cam portion of each of the one or more retention members is axially spaced from the respective curved surface of the non-pivoting strut, which allows the actuation member to move axially relative to the one or more retention members toward the second end portion of the frame.

Example 178. The prosthetic heart valve of any example herein, particularly example 177, wherein the cam portion of each of the one or more retention members moves laterally relative to the actuation member when the one or more retention members move between the locked position to the unlocked position.

Example 179. The prosthetic heart valve of any example herein, particularly example 177 or example 178, wherein the cam portion of each of the one or more retention members contacts the actuation member in the locked position and the unlocked position.

Example 180. The prosthetic heart valve of any example herein, particularly any one of examples 177-179, wherein in the locked position the arm portion of each of the retention members is in an axially-elongate state and the cam portion of each of the retention members is in a laterally-compressed state, and wherein in the unlocked position the arm portion of each of the retention members is in an axially-compressed state and the cam portion of each of the retention members is in a laterally-expanded state.

Example 181. The prosthetic heart valve of any example herein, particularly example 180, wherein the arm portion of each of the retention members is biased to the axially-elongate state.

Example 182. The prosthetic heart valve of any example herein, particularly example 180 or example 181, wherein the cam portion is biased to the laterally-expanded state.

Example 183. The prosthetic heart valve of any example herein, particularly any one of examples 177-182, wherein the cam portion of each of the one or more retention members pivots relative to a respective arm portion when the one or more retention members move between the locked position and the unlocked position.

Example 184. The prosthetic heart valve of any example herein, particularly any one of examples 177-183, wherein the actuation member comprises a plurality of projections and a plurality of notches, and wherein the cam portion of each of the one or more retention members is configured to engage the plurality of projections and the plurality of notches.

Example 185. The prosthetic heart valve of any example herein, particularly any one of examples 177-184, wherein the actuation member comprises threads disposed on an outer surface.

Example 186. The prosthetic heart valve of any example herein, particularly any one of examples 177-185, wherein the cam portion of each of the one or more retention members comprises a tooth configured to engage the actuation member.

Example 187. The prosthetic heart valve of any example herein, particularly example 186, wherein the tooth of each cam portion can slide axially over the threads of the actuation member when the one or more retention members are in the unlocked position, thereby allowing the frame to expand radially.

Example 188. The prosthetic heart valve of any example herein, particularly any one of examples 177-187, wherein the actuation member is rotatable relative to the one or more retention members.

Example 189. The prosthetic heart valve of any example herein, particularly example 188, wherein when the one or more retention members are in the locked position, rotating the actuation member in a first direction relative to the one or more retention members results in radial compression of the prosthetic heart valve.

Example 190. The prosthetic heart valve of any example herein, particularly any one of examples 177-189, wherein each arm of the one or more retention members is a spring.

Example 191. A method of implanting a prosthetic heart valve, comprising inserting a prosthetic heart valve into a patient's vasculature, the prosthetic heart valve releasably coupled to a distal end portion of a delivery apparatus and in a first radially-compressed configuration, advancing the prosthetic heart valve through the patient's vasculature to an implantation location, expanding the prosthetic heart valve to a first radially-expanded configuration by moving an actuation member of the prosthetic heart valve in a first axial direction relative to a locking mechanism of the prosthetic heart valve, wherein the actuation member is restricted from rotating relative to the locking mechanism during the expansion of the prosthetic heart valve to the first radially-expanded configuration, locking the prosthetic heart valve in the first radially-expanded configuration by engaging the actuation member with the locking mechanism of the prosthetic heart valve such that the actuation member is restricted from moving in a second axial direction relative to the locking mechanism, and compressing the prosthetic heart valve from the first radially-expanded configuration to a second radially-compressed configuration, which is larger than the first radially-compressed configuration, wherein compressing the prosthetic heart valve includes rotating the actuation member in a first rotational direction relative to the locking mechanism.

Example 192. The method of any example herein, particularly example 191, wherein prior to expanding the prosthetic heart valve to the first radially-expanded configuration, the method further comprises allowing the prosthetic heart valve to self-expand from the first radially-compressed configuration to a partially radially-expanded configuration, which is smaller than the first radially-expanded configuration.

Example 193. The prosthetic heart valve of any example herein, particularly example 191 or example 192, further comprising: expanding the prosthetic heart valve from the second radially-compressed configuration to a second radially-expanded configuration by moving the actuation member of the prosthetic heart valve in the first axial direction relative to the locking mechanism of the prosthetic heart valve, wherein the actuation member is restricted from rotating relative to the locking mechanism during the expansion of the prosthetic heart valve to the second radially-expanded configuration.

Example 194. The method of any example herein, particularly example 193, wherein the second radially-expanded configuration is smaller than the first radially-expanded configuration.

Example 195. The method of any example herein, particularly example 193, wherein the second radially-expanded configuration is larger than the first radially-expanded configuration.

Example 196. The method of any example herein, particularly any one of examples 191-195, further comprising moving the prosthetic heart valve relative to the patient's vasculature while the prosthetic heart valve is in the second radially-compressed configuration.

Example 197. The method of any example herein, particularly any one of examples 191-196, wherein the locking mechanism comprises a chamber and a retention member, wherein the chamber is formed in a non-pivoting strut of a frame of the prosthetic heart valve, and wherein the retention member comprises an arm portion extending from the non-pivoting strut of the frame and a cam portion extending from the arm portion and configured to engage the actuation member.

Example 198. The method of any example herein, particularly example 197, wherein the cam portion engages the actuation member when the locking mechanism is in a locked position and in an unlocked position.

Example 199. The method of any example herein, particularly example 197 or example 198, wherein expanding the prosthetic heart valve to the first radially-expanded configuration includes axially compressing the arm portion of the locking mechanism.

Example 200. The method of any example herein, particularly any one of examples 191-199, wherein the actuation member is one actuation member of a plurality of actuation members, wherein the locking mechanism is one locking mechanism of a plurality of locking mechanisms, and wherein expanding the prosthetic heart valve to the first radially-expanded configuration includes moving each of the actuation members in the first axial direction relative to a respective locking mechanism.

Example 201. The method of any example herein, particularly any one of examples 191-200, further comprising releasing the prosthetic heart valve from the delivery apparatus by rotating an actuation shaft of the delivery apparatus in a second rotational direction relative to the actuation member of the prosthetic heart valve.

Example 202. The method of any example herein, particularly any one of examples 191-200, further comprising releasing the prosthetic heart valve from the delivery apparatus by moving an actuation shaft of the delivery apparatus in the second axial direction relative to a locking shaft of the delivery apparatus and the actuation member of the prosthetic heart valve, and moving the actuation shaft and the locking shaft in the first axial direction relative to the actuation member of the prosthetic heart valve.

Example 203. A prosthetic heart valve comprising a plurality of pivoting struts, a plurality of non-pivoting struts, an actuation member, and a locking mechanism. The plurality of non-pivoting struts includes a first non-pivoting strut and a second non-pivoting strut axially spaced apart from each other. The plurality of non-pivoting struts is fixedly coupled to the plurality of pivoting struts. The second non-pivoting strut includes a lumen. The actuation member fixedly coupled to the first non-pivoting strut and extending from the first non-pivoting strut and into the lumen of the second non-pivoting strut. The locking mechanism comprises a chamber and a retention member. The chamber is formed in the second non-pivoting strut, intersects with the lumen of the second non-pivoting strut, and is configured to receive the retention member therein. The prosthetic heart valve is radially expandable from a radially-compressed state to a radially-expanded state by moving the actuation member in a first axial direction relative to the second non-pivoting strut. The prosthetic heart valve is radially compressible from the radially-expanded state to the radially-compressed state by moving the actuation member in a second axial direction relative to the second non-pivoting strut. The locking mechanism is movable within the chamber from a locked position to an unlocked position. In the locked position, the retention member engages the actuation member, prevents the actuation member from moving in the second axial direction to the second non-pivoting strut, and allows the actuation member to move in the first axial direction relative to the second non-pivoting strut. In unlocked position, the retention member is disengaged from the actuation member and movable in the first axial direction relative to the second non-pivoting strut.

Example 204. The prosthetic heart valve of any example herein, particularly example 203, wherein the plurality of pivoting struts and the plurality of non-pivoting struts are integrally formed as a single, unitary component.

Example 205. The prosthetic heart valve of any example herein, particularly example 203 or example 204, wherein the chamber comprises a first end portion and a second end portion, the first end portion of the chamber being disposed closer to the first non-pivoting strut than the second end portion of the chamber, and wherein the retention member is disposed closer to the first end portion of the chamber when the locking mechanism is in the locked position than when the locking mechanism is in the unlocked position.

Example 206. The prosthetic heart valve of any example herein, particularly any one of examples 203-205, wherein the chamber intersects a larger portion of the lumen of the second non-pivoting strut when the locking mechanism is in the locked position than when the locking mechanism is in the unlocked position.

Example 207. The prosthetic heart valve of any example herein, particularly any one of examples 203-206, wherein the lumen of the second non-pivoting strut comprises a first longitudinal axis, and wherein the chamber comprises a second longitudinal axis, which is oblique relative to the first longitudinal axis.

Example 208. The prosthetic heart valve of any example herein, particularly any one of examples 203-207, wherein the locking mechanism is biased to the locked position.

Example 209. The prosthetic heart valve of any example herein, particularly example 208, further comprising a spring coupled to the retention member and configured to bias the retention member against the actuation member.

Example 210. The prosthetic heart valve of any example herein, particularly any one of examples 203-209, wherein the retention member comprises one or more arm portions and a hook portion, wherein the one or more arm portions are configured for movably coupling the retention member to the second non-pivoting strut, and wherein the hook portion is configured to engage the actuation member when the locking mechanism is in the locked position.

Example 211. The prosthetic heart valve of any example herein, particularly any one of examples 203-210, wherein the chamber comprises a width that is less than a combined width of the actuation member and the retention member.

Example 212. The prosthetic heart valve of any example herein, particularly any one of examples 203-211, wherein the actuation member comprises one or more projections configured for receiving the retention member therebetween.

Example 213. The prosthetic heart valve of any example herein, particularly any one of examples 203-212, wherein the actuation member comprises one or more grooves configured for receiving the retention member therein.

Example 214. A delivery apparatus for a prosthetic implant, comprising a handle, a locking shaft, and an actuation shaft. The locking shaft having a proximal end portion and a distal end portion. The proximal end portion of the locking shaft is movably coupled to the handle. The distal end portion of the locking shaft is configured to be inserted through a lumen of a prosthetic implant having a diameter and to be movable between a straight configuration and a flared configuration. In the straight configuration, the distal end portion of the locking shaft has a first outer diameter and a first inner diameter, the first outer diameter being less than the diameter of the lumen. In the flared configuration, the distal end portion of the locking shaft has a second outer diameter and a second inner diameter, the second outer diameter being greater than the diameter of the lumen. The actuation shaft extending coaxially through the locking shaft and having a proximal end portion and a distal end portion, wherein the proximal end portion of the actuation shaft is movably coupled to the handle. The distal end portion of the actuation shaft has an outer diameter that is less than the diameter of the lumen, less than the second inner diameter of the locking shaft, and greater than the first inner diameter of the locking shaft. The actuation shaft and the locking shaft are axially movable relative to each other between an engaged state and a disengaged state. In the engaged state, the distal end portion of the locking shaft is in the flared configuration and the distal end portion of the actuation shaft is at least partially disposed within the locking shaft such that an outer surface of the actuation shaft contacts an inner surface of the distal end portion of the locking shaft. The actuation shaft secures the locking shaft in the flared configuration. The locking shaft prevents the actuation shaft from moving proximally relative to the locking shaft. In the disengaged state, the distal end portion of the actuation shaft is positioned distal relative to the distal end portion of the locking shaft such that the outer surface of the actuation shaft is spaced from the inner surface of the distal end portion of the locking shaft. The locking shaft can move from the flared configuration to the straight configuration. The locking shaft can move proximally relative to the actuation shaft.

Example 215. The delivery apparatus of any example herein, particularly example 214, further comprising a support sleeve, wherein the locking shaft and the actuation shaft extend coaxially through the support sleeve.

Example 216. The delivery apparatus of any example herein, particularly example 214 or example 215, wherein the handle comprises a first knob and a main body, wherein the first knob is coupled to the actuation shaft and is configured such that rotating the first knob in a first rotational direction relative to the main body moves the actuation shaft axially distally relative to the locking shaft and such that rotating the first knob in a second rotational direction relative to the main body moves the actuation shaft axially proximally relative to the locking shaft.

Example 217. The delivery apparatus of any example herein, particularly example 216, wherein the handle comprises a second knob, wherein the second knob is coupled to the locking shaft and configured such that rotating the second knob in the first rotational direction relative to the main body moves the locking shaft axially distally relative to the actuation shaft and such that rotating the second knob in the second rotational direction relative to the main body moves the locking shaft axially proximally relative to the actuation shaft.

Example 218. The delivery apparatus of any example herein, particularly any one of examples 214-217, further comprising a first shaft and a second shaft, wherein the first shaft comprises a first end portion coupled to the handle and a second end portion having a delivery capsule configured for receiving the prosthetic implant therein, and wherein the second shaft extends through the first shaft and comprises a lumen configured for receiving the locking shaft and the actuation shaft therein.

Example 219. The delivery apparatus of any example herein, particularly any one of examples 214-218, wherein the locking shaft is a first locking shaft of a plurality of locking shafts, and wherein the actuation shaft is a first actuation shaft of a plurality of actuation shafts.

Example 220. The delivery apparatus of any example herein, particularly any one of examples 214-219, wherein the plurality of locking shafts comprises 2-15 locking shafts, and wherein the plurality of actuation shafts comprises 2-15 actuation shafts.

Example 221. The delivery apparatus of any example herein, particularly any one of examples 214-219, wherein the plurality of locking shafts comprises 3-12 locking shafts, and wherein the plurality of actuation shafts comprises 3-12 actuation shafts.

Example 222. The delivery apparatus of any example herein, particularly any one of examples 214-219, wherein the plurality of locking shafts comprises 6-9 locking shafts, and wherein the plurality of actuation shafts comprises 6-9 actuation shafts.

Example 223. The delivery apparatus of any example herein, particularly any one of examples 214-219, wherein the plurality of locking shafts comprises exactly six locking shafts, and wherein the plurality of actuation shafts comprises exactly six actuation shafts.

Example 224. The delivery apparatus of any example herein, particularly any one of examples 214-223, wherein the locking shaft comprises a tube formed of a polymeric material.

Example 225. The delivery apparatus of any example herein, particularly any one of examples 214-223, wherein the locking shaft comprises a tube formed of a metallic material.

Example 226. The delivery apparatus of any example herein, particularly any one of examples 214-225, wherein the actuation shaft comprises a metal shaft.

Example 227. The delivery apparatus of any example herein, particularly any one of examples 214-225, wherein the actuation shaft comprises a metal wire.

Example 228. The delivery apparatus of any example herein, particularly any one of examples 214-225, wherein the actuation shaft comprises a metal cable.

Example 229. The delivery apparatus of any example herein, particularly any one of examples 214-225, wherein the actuation shaft comprises a metal rod.

Example 230. The delivery apparatus of any example herein, particularly any one of examples 214-229, wherein the distal end portion of the actuation shaft comprises a stopper fixedly coupled thereto.

Example 231. The delivery apparatus of any example herein, particularly example 230, wherein the stopper is integrally formed with the actuation shaft.

Example 232. The delivery apparatus of any example herein, particularly example 230, wherein the stopper is fixedly coupled to the actuation shaft via one or more of a frictional engagement, an adhesive, a fastener, or welding.

Example 233. A delivery assembly comprising the delivery apparatus of any example herein, particularly any one of examples 214-232, and a prosthetic heart valve comprising a radially-expandable frame. The frame of the prosthetic heart valve is releasably coupled to the delivery apparatus by the actuation shaft and the locking shaft of the delivery apparatus.

Example 234. A delivery assembly comprising the delivery apparatus of any example herein, particularly any one of examples 214-232, and a radially-expandable stent. The stent is releasably coupled to the delivery apparatus by the actuation shaft and the locking shaft of the delivery apparatus.

Example 235. A method of implanting a prosthetic implant, comprising positioning an actuation shaft of a delivery apparatus through a lumen of a prosthetic implant such that a distal end portion of the actuation shaft is disposed distal to a distal end of the lumen, positioning a locking shaft of the delivery apparatus over the actuation shaft and through the lumen of the prosthetic implant such that a distal end portion of the locking shaft is disposed distal to the distal end of the lumen and proximal to the distal end portion of the actuation shaft, wherein the distal end portion of the locking shaft comprises a flange that flares radially and contacts the prosthetic implant, moving the actuation shaft proximally relative to the locking shaft such that the distal end portion of the actuation shaft contacts the flange of the locking shaft such that the actuation shaft and the locking shaft are restricted from moving proximally relative to the prosthetic implant, inserting the prosthetic implant into a patient's body together with the distal end portion of the actuation shaft and the distal end portion of the locking shaft, advancing the prosthetic implant to an implantation location with the patient's body, expanding the prosthetic implant from a radially-compressed configuration to a radially-expanded configuration by applying an axially-compressive force on the prosthetic implant via the actuation shaft, locking the prosthetic implant in the radially-expanded configuration with a locking mechanism of the prosthetic implant, and releasing the prosthetic implant from the delivery apparatus by moving the distal end portion of the actuation shaft distally relative to the locking shaft, moving the locking shaft proximally relative to the prosthetic implant such the locking shaft is withdrawn from the lumen, and moving the actuation shaft proximally relative to the prosthetic implant such that the actuation shaft is withdrawn from the lumen.

Example 236. A frame for a prosthetic heart valve, comprising a plurality of pivoting struts, a plurality of non-pivoting struts coupled to the plurality of pivoting struts, an actuation member coupled to a first non-pivoting strut of the plurality of non-pivoting struts, and a locking mechanism coupled to a second non-pivoting strut of the plurality of non-pivoting struts. The locking mechanism is integrally formed as a single, unitary component with the plurality of pivoting struts and the plurality of non-pivoting struts. The frame is movable from a radially-compressed state to a radially-expanded state. In the radially-compressed state, the actuation member is axially spaced from the locking mechanism. In the radially-expanded state, locking mechanism engages the actuation member and prevents the frame from moving from the radially-expanded state to the radially-compressed state.

Example 237. A prosthetic heart valve comprising the frame or the stent of any example herein, particularly any one of examples 1-236, and a valve structure coupled to the frame and comprising a plurality of leaflets configured for allowing blood to flow in an antegrade direction and to restrict blood from flowing in a retrograde direction.

Example 238. The prosthetic heart valve of any example herein, particularly example 236 or example 237, further comprising one or more sealing skirts coupled to the frame and configured for reducing paravalvular leakage.

Example 239. A delivery assembly comprising the prosthetic heart valve of any example herein, particularly any one of examples 236-238, and a delivery apparatus releasably coupled to the prosthetic heart valve.

Example 240. The delivery assembly of any example herein, particularly example 239, wherein the delivery apparatus is releasably coupled to the prosthetic heart valve via a threaded connection.

Example 241. The delivery assembly of any example herein, particularly example 239, wherein the delivery apparatus is releasably coupled to the prosthetic heart valve via a suture.

Example 242. The delivery assembly of any example herein, particularly example 240, wherein the delivery apparatus is releasably coupled to the prosthetic heart valve via a plurality of interlocking shafts.

Example 243. A method of implanting a prosthetic heart valve, comprising releasably coupling the frame or the stent of any example herein, particularly any one of examples 1-236, to a distal end portion of a delivery apparatus, applying an axially-compressive force on the frame to radially expand the frame, locking the frame in a final radially-expanded state with the locking mechanism of the frame, releasing the frame from the delivery apparatus.

Example 244. The method of any example herein, particularly example 243, wherein prior to applying the axially-compressive force on the frame, the method further comprises allowing the frame or the stent to radially self-expand.

Example 245. The method of any example herein, particularly example 243 or example 244, wherein after applying the axially-compressive force on the frame and prior to locking the frame in the final radially-expanded state, the method further comprises radially compressing the frame.

Example 246. The method of any example herein, particularly any one of examples 243-245, wherein prior to locking the frame in the final radially-expanded state, the method further comprises locking the frame in an initial radially-expanded state, wherein the initial radially-expanded state is smaller than the final radially-expanded state.

The features described herein with regard to any example can be combined with other features described in any one or more of the other examples, unless otherwise stated. For example, any one or more of the features of one locking mechanism can be combined with any one or more features of another locking mechanism. As another example, any one or more features of one delivery apparatus can be combined with any one or more features of another delivery apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A prosthetic heart valve comprising:
a frame comprising a plurality of struts, an actuation member, a locking mechanism, a first end portion, and a second end portion, wherein the frame is movable from a radially-compressed configuration to a plurality of radially-expanded configurations, wherein the plurality of struts and the locking mechanism are integrally formed as a unitary component, wherein the actuation member extends from the first end portion of the frame toward the second end portion of the frame, wherein the locking mechanism is disposed at the second end portion of the frame and is configured to receive the actuation member, and wherein the locking mechanism is configured to selectively engage the actuation member such that the actuation member is movable in a first direction relative to the frame when the frame is in a first radially-expanded configuration, thereby allowing further radial expansion of the frame from the first radially-expanded configuration to a second radially-expanded configuration, and such that the actuation member is prevented from moving in a second direction relative to the frame when the frame is in the first radially-expanded configuration, thereby preventing the frame from moving from the first radially-expanded configuration to the radially-compressed configuration.

2. The prosthetic heart valve of claim 1, further comprising a valve structure coupled to the frame, wherein the valve structure comprises a plurality of leaflets configured for allowing blood to flow in an antegrade direction and to restrict blood from flowing in a retrograde direction.

3. The prosthetic heart valve of claim 1, wherein the actuation member of the frame comprises a first threaded portion configured for threadably coupling the actuation member to a second threaded portion of a delivery apparatus.

4. The prosthetic heart valve of claim 1, wherein the frame comprises a lumen configured for receiving a plurality of shafts of a delivery apparatus configured for releasably coupling the prosthetic heart valve to the delivery apparatus via a non-threaded connection.

5. The prosthetic heart valve of claim 1, wherein the actuation member, the plurality of struts, and the locking mechanism are integrally formed as a unitary component.

6. The prosthetic heart valve of claim 1, wherein the plurality of struts of the frame comprises a plurality of pivoting struts and a plurality of non-pivoting struts, wherein the pivoting struts are integrally formed with or fixedly coupled to the non-pivoting struts, and wherein the pivoting struts pivot relative to the non-pivoting struts as the frame moves from the radially-compressed configuration to the first radially-expanded configuration.

7. The prosthetic heart valve of claim 6, wherein the actuation member is coupled to a first non-pivoting strut of the plurality of non-pivoting struts, which is disposed at the first end portion of the frame, and wherein the locking mechanism is formed in a second non-pivoting strut of the plurality of non-pivoting struts, which is disposed at the second end portion of the frame.

8. The prosthetic heart valve of claim 1, wherein the actuation member comprises an elongate shaft.

9. The prosthetic heart valve of claim 1, wherein the actuation member comprises an elongate rod.

10. The prosthetic heart valve of claim 1, wherein the actuation member comprises a circular cross-sectional profile taken in a plane perpendicular to a longitudinal axis of the actuation member.

11. The prosthetic heart valve of claim 1, wherein the actuation member comprises a non-circular cross-sectional profile taken in a plane perpendicular to a longitudinal axis of the actuation member.

12. A prosthetic heart valve comprising:
a frame comprising a plurality of struts, a plurality of actuation members, a plurality of locking mechanisms, a first end portion and a second end portion, wherein the frame is movable from a radially-compressed configuration to a plurality of radially-expanded configurations, wherein the plurality of struts and the plurality of locking mechanisms are integrally formed as a unitary component, wherein the plurality of actuation members extends from the first end portion of the frame toward the second end portion of the frame, each actuation member of the plurality of actuation members being spaced circumferentially relative to an adjacent actuation member of the plurality of actuation members, wherein the plurality of locking mechanisms is disposed at the second end portion of the frame, each locking mechanism of the plurality of locking mechanisms being spaced circumferentially relative to an adjacent locking mechanism of the plurality of actuation members and configured to receive a respective actuation member of the plurality of actuation members and to selectively engage the respective actuation member such that the respective actuation member is movable in a first direction relative to the frame when the frame is in a first radially-expanded configuration, thereby allowing the frame to radially expand from the first radially-expanded configuration to a second radially-expanded configuration, and such that the respective actuation member is prevented from moving in a second direction relative to the frame when the frame is in the first radially-expanded configuration, thereby preventing the frame from moving from the first radially-expanded configuration to the radially-compressed configuration.

13. The prosthetic heart valve of claim 12, wherein the plurality of actuation members comprises 2-15 actuation members, and wherein the plurality of locking mechanisms comprises 2-15 locking mechanisms.

14. The prosthetic heart valve of claim 12, wherein the plurality of actuation members comprises 3-12 actuation members, and wherein the plurality of locking mechanisms comprises 3-12 locking mechanisms.

15. The prosthetic heart valve of claim 12, wherein the plurality of actuation members comprises 6-9 actuation members, and wherein the plurality of locking mechanisms comprises 6-9 locking mechanisms.

16. The prosthetic heart valve of claim 12, wherein the plurality of actuation members comprises exactly three actuation members, and wherein the plurality of locking mechanisms comprises exactly three locking mechanisms.

17. The prosthetic heart valve of claim 12, wherein the plurality of actuation members comprises exactly six actuation members, and wherein the plurality of locking mechanisms comprises exactly six locking mechanisms.

18. The prosthetic heart valve of claim 12, wherein the plurality of actuation members comprises exactly nine actuation members, and wherein the plurality of locking mechanisms comprises exactly nine locking mechanisms.

19. A method of implanting a prosthetic heart valve, comprising:
inserting a prosthetic heart valve into a patient's vasculature, the prosthetic heart valve releasably coupled to a distal end portion of a delivery apparatus and in a radially-compressed configuration;
advancing the prosthetic heart valve through the patient's vasculature to an implantation location;
expanding the prosthetic heart valve to a radially-expanded configuration by applying an axially-compressive force on the prosthetic heart valve with the delivery apparatus; and
locking the prosthetic heart valve in the radially-expanded configuration by engaging an actuation member and a locking mechanism of the prosthetic heart valve such that the prosthetic heart valve is prevented from moving from the radially-expanded configuration to the radially-compressed configuration, wherein the locking mechanism is integrally formed with a frame of the prosthetic heart valve.

20. The method of claim 19, wherein the actuation member is integrally formed with the frame and the locking mechanism.
